(19) United States
(12) Patent Application Publication
(10) Pub. No.: US 2017/0105997 A1
(43) Pub. Date: Apr. 20, 2017

(54) METHODS OF TREATING CANCER

(71) Applicants: Northwestern University, Evanston, IL (US); GlaxoSmithKline Intellectual Property (No.2) Limited, Brentford, Middlesex (GB)

(72) Inventors: Caretha L. CREASY, Collegeville, PA (US); Jonathan LIGHT, Evanston, IL (US); Michael McCABE, Collegeville, PA (US); Relja POPOVIC, Evanston, IL (US)

(21) Appl. No.: 15/127,873

(22) PCT Filed: Mar. 21, 2015
(86) PCT No.: PCT/US15/21905

§ 371 Date: Sep. 21, 2016

Related U.S. Application Data
(60) Provisional application No. 61/968,473, filed on Mar. 21, 2014.
(60) Provisional application No. 61/968,650, filed on Mar. 21, 2014.
(60) Provisional application No. 61/968,478, filed on Mar. 21, 2014.

(51) Int. Cl. A61K 31/5377; A61K 31/496; A61K 45/06

(52) U.S. Cl. CPC A61K 31/5377; A61K 45/06; A61K 31/496

(57) ABSTRACT

Disclosed are methods for treating cancer such as a myeloid malignancy such as multiple myeloma in a human using EZH2 inhibitors in human populations having a translocation MMSET or a decreased level of a functional UTX protein or both.

### FIELD

This invention relates to methods of treating cancer in a subject in need thereof.

### BACKGROUND

The expanding development and use of targeted therapies for cancer treatment reflects an increasing understanding of key oncogenic pathways, and how the targeted perturbation of these pathways corresponds to clinical response. Difficulties in predicting efficacy to targeted therapies is likely a consequence of the limited global knowledge of causal mechanisms for pathway deregulation (e.g. activating mutations, amplifications). Pre-clinical translational research studies for oncology therapies focuses on determining what tumor type and genotypes are most likely to benefit from treatment. Treating selected patient populations may help maximize the potential of a therapy. Pre-clinical cellular response profiling of tumor models has become a cornerstone in development of novel cancer therapeutics. Efforts to predict clinical efficacy using cohorts of in vitro tumor models have been successful (e.g. EGFR inhibitors are selectively useful in those tumors harboring EGFR mutations). Thus, expansive panels of diverse tumor derived cell lines could recapitulate an ‘all comers’ efficacy trial; thereby identifying which histologies and specific tumor genotypes are most likely to benefit from treatment. Numerous specific molecular markers are now used to identify patients most likely to benefit in a clinical setting.

EZH2 (enhancer of zeste homolog 2; human EZH2 gene: Cardoso, C, et al; European J of Human Genetics, Vol. 8, No. 3 Pages 174-180, 2000) is the catalytic subunit of the Polycomb Repressor Complex 2 (PRC2) which functions to silence target genes by tri-methylating lysine 27 of histone H3 (H3K27me3). Histone H3 is one of the five main histone proteins involved in the structure of chromatin in eukaryotic cells. Featuring a main globular domain and a long N-terminal tail, Histones are involved with the structure of the nucleosomes, a ‘beads on a string’ structure. Histone proteins are highly post-translationally modified however Histone H3 is the most extensively modified of the five histones. The term “Histone H3” alone is purposely ambiguous in that it does not distinguish between sequence variants or modification state. Histone H3 is an important protein in the emerging field of epigenetics, where its sequence variants and variable modification states are thought to play a role in the dynamic and long term regulation of genes.

EZH2 inhibitors that are useful in treating cancer have been reported in PCT applications PCT/US2011/035336, PCT/US2011/035340, and PCT/US2011/035344, and U.S. Pat. No. 8,410,088, each of which are incorporated by reference herein. It is desirable to identify characteristics of tumors and or subpopulations (e.g. genotypes) that are more likely to respond to these compounds.

### SUMMARY OF THE INVENTION

1. A method of treating cancer in a human in need thereof, comprising determining at least one of the following in a sample from said human:
   a. the presence or absence of a t(4,14) translocation in MMSET as compared to a control; or
   b. the presence or absence of a decreased level of a functional UTX protein as compared to a control; and
      and administering to said human an effective amount of an EZH2 inhibitor or pharmaceutically acceptable salt thereof if the t(4,14) translocation in MMSET is present, or there is a decreased level of a functional UTX protein as compared to a control, or both.

### BRIEF DESCRIPTION OF THE DRAWINGS

FIG. 1. MMSET overexpression alters normal H3K36me2 distribution

(A) Loss of MMSET expression in t(4;14)+ cells depletes H3K36 methylation and increases H3K27 methylation. Immunoblot of nuclear extracts from t(4;14)+ KMS11 cells with inactivated translocated (TKO) or wild-type (NTKO) MMSET allele were probed using antibodies for MMSET, H3K36me2, H3K27me3 and total H4 as control. (B) Heatmaps of H3K36me2 distribution between NTKO (left) and TKO (right) cells. On the y-axis, genes were ranked based on their expression level from high (top) to low (bottom) and on the x-axis, normalized read density from ChIP-seq experiments was plotted in gene bodies and 10 kb upstream and downstream from the transcription start (TSS) and end (TES) sites. (C) MMSET alters H3K36me2 distribution in intragenic (top) and intergenic (bottom) regions. (Top) Average read density across 15,386 genes in NTKO (left, red) and TKO (right, green) cells. (Middle) UCSC genome browser view of the H3K36me2 distribution on a representative locus (BTF3) in NTKO and TKO cells. (Bottom) Average normalized tag density of H3K36me2 in 6,172 intergenic regions in NTKO (left) and TKO (right) cells. (D) Representative UCSC genome browser view of the H3K36me2 distribution on chromosome 8 of NTKO (red) and TKO (green) cells. Top panel encompasses a gene-rich region of the chromosome and the bottom insert represents a 1 MB region of the 8q24 gene desert.

FIG. 2. High levels of MMSET lead to altered gene expression

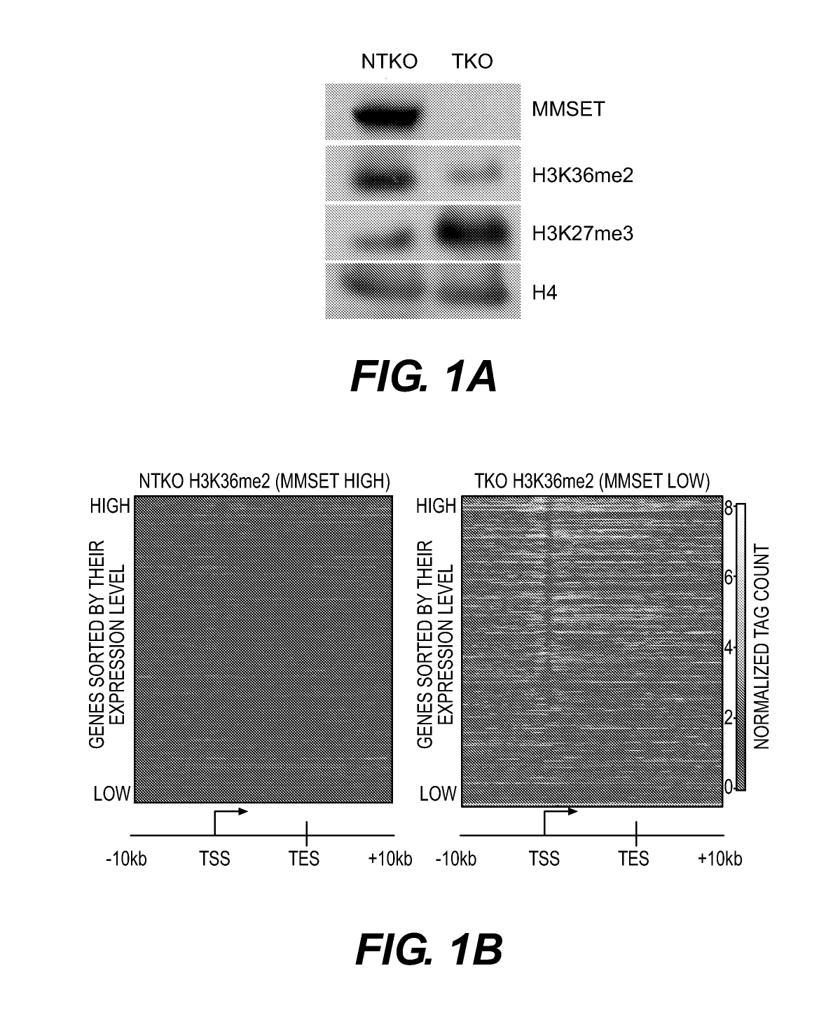

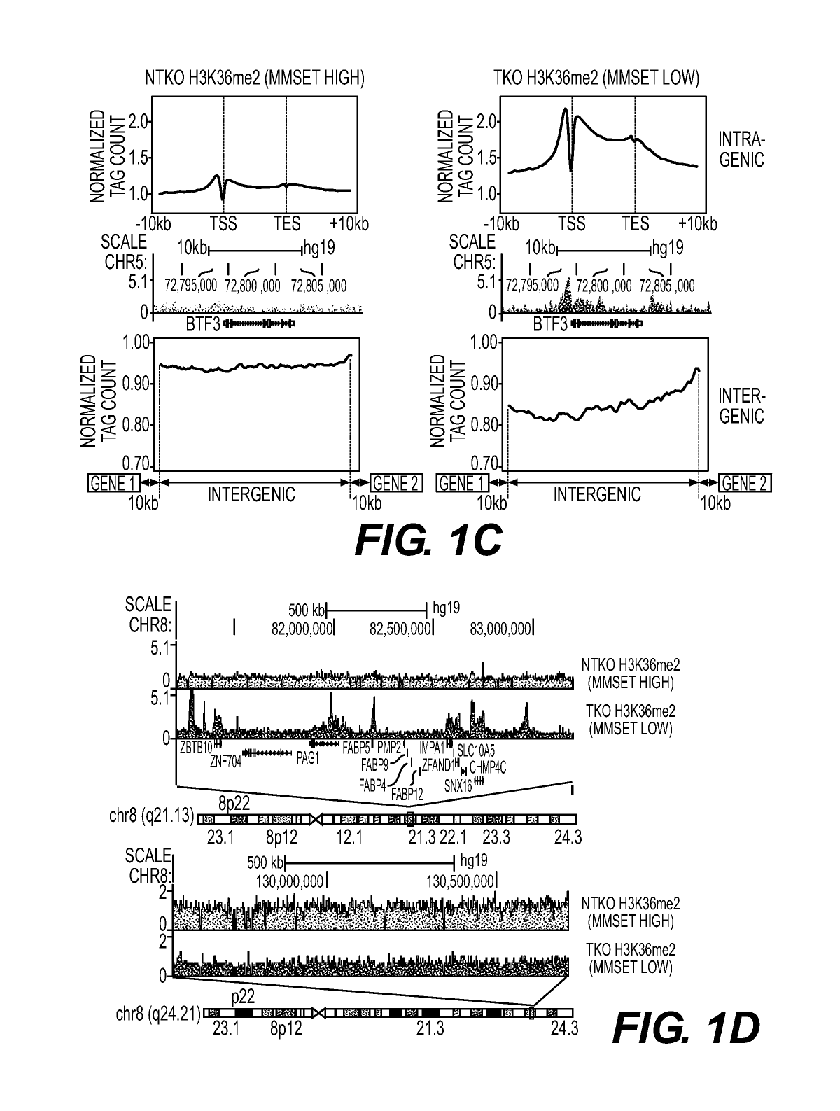

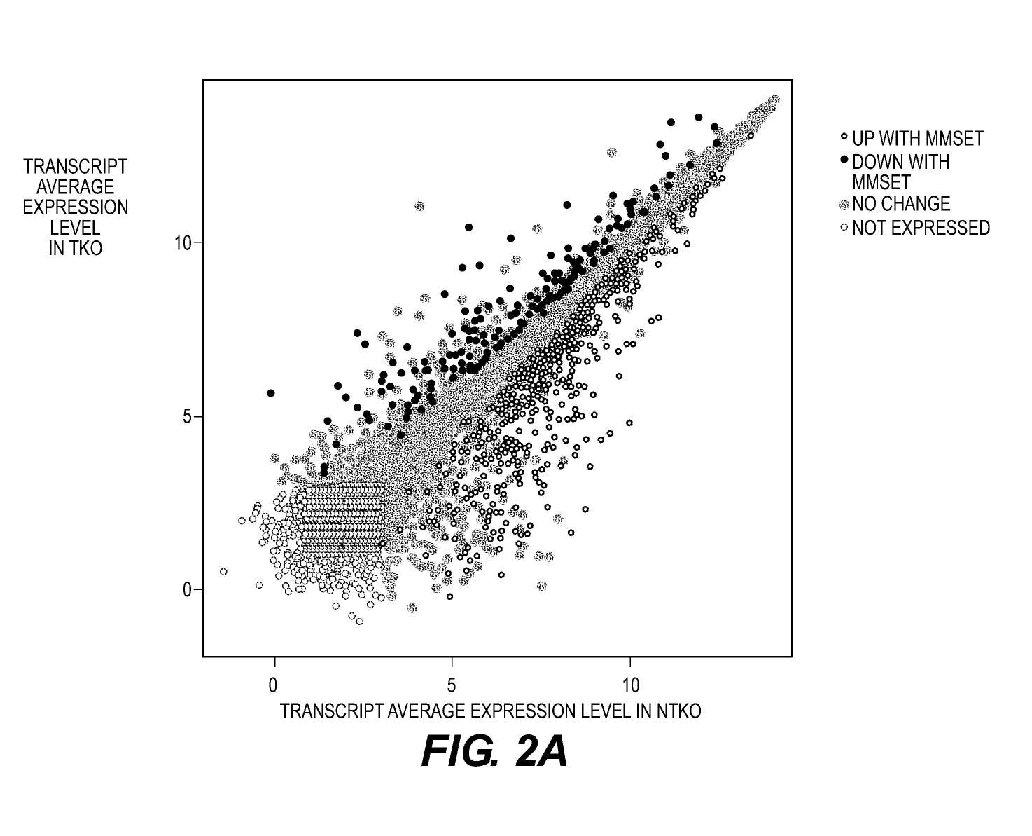

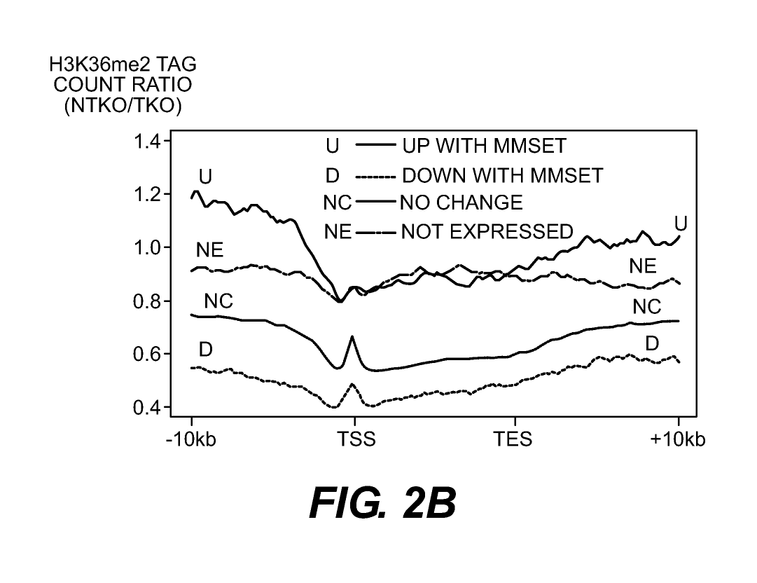

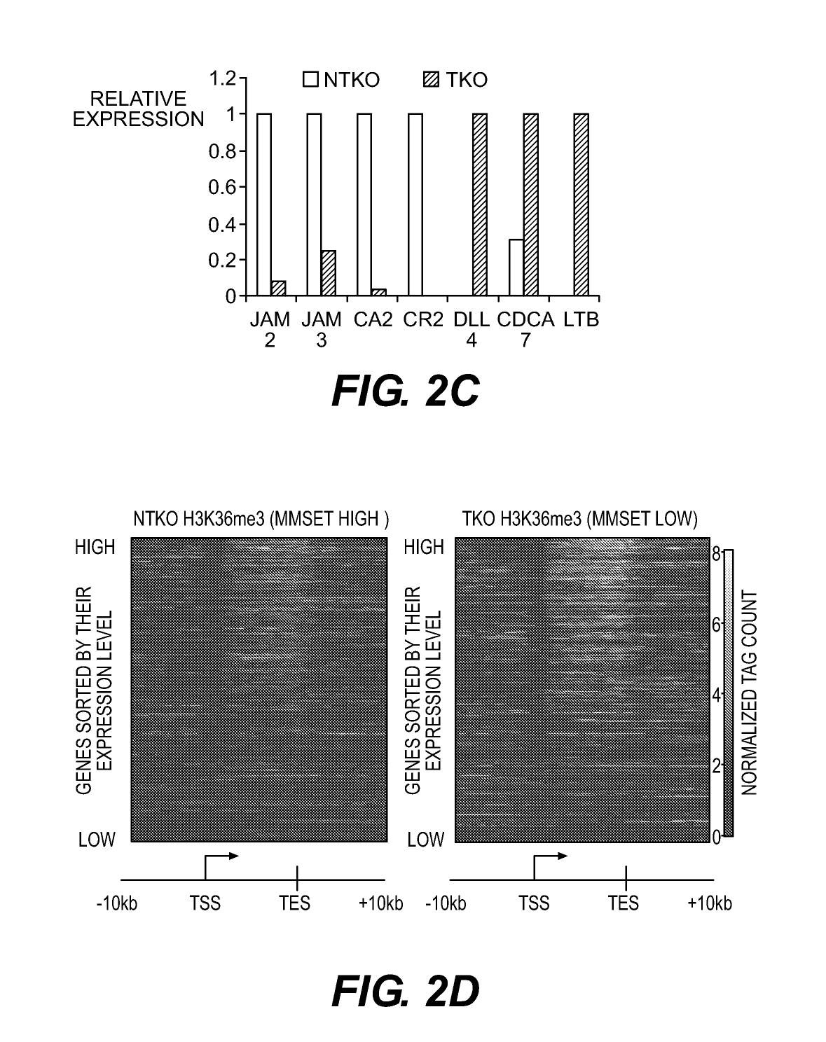

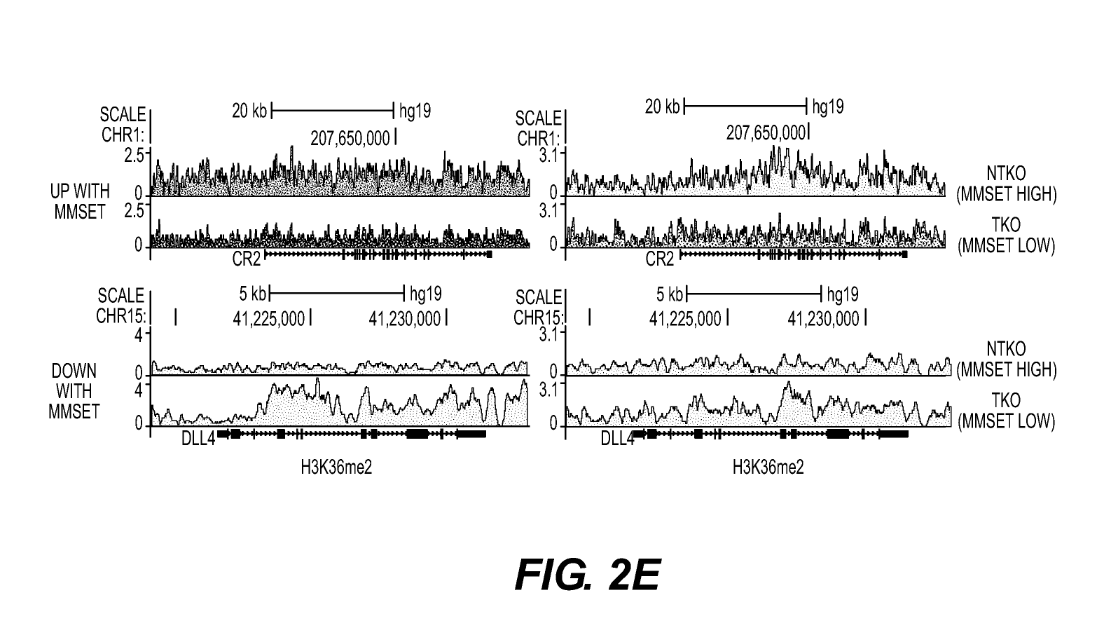

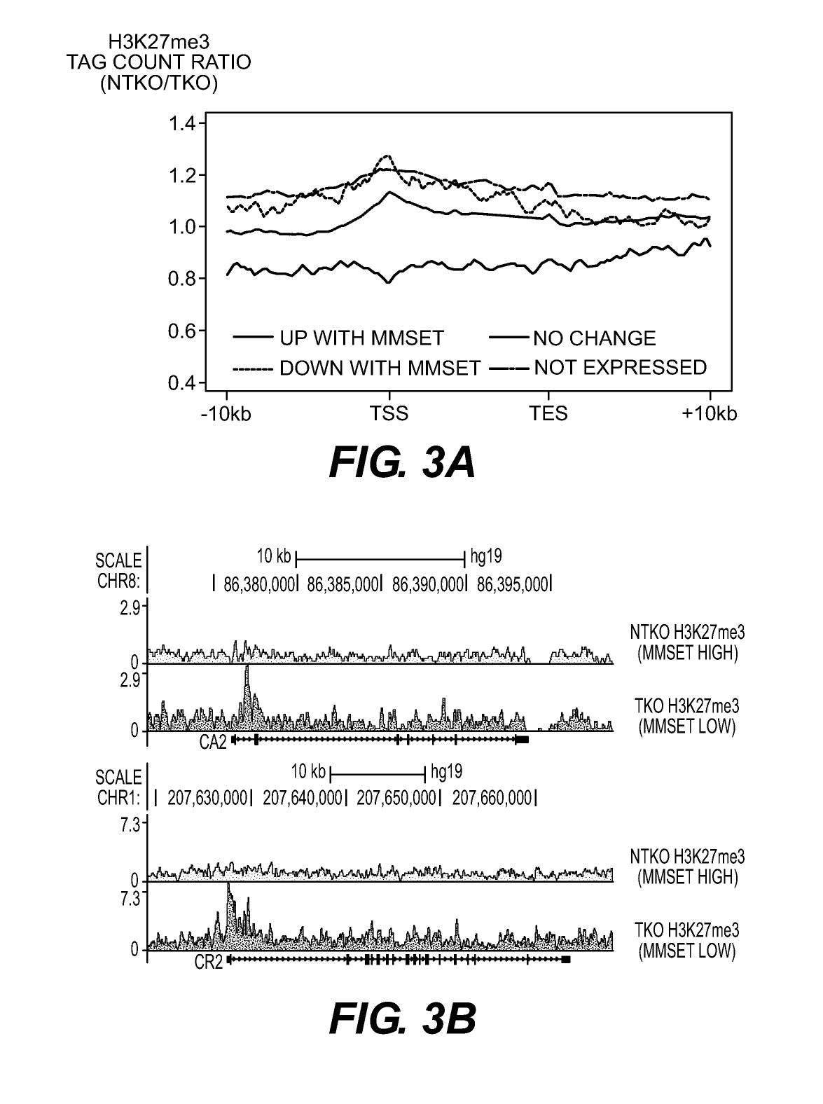

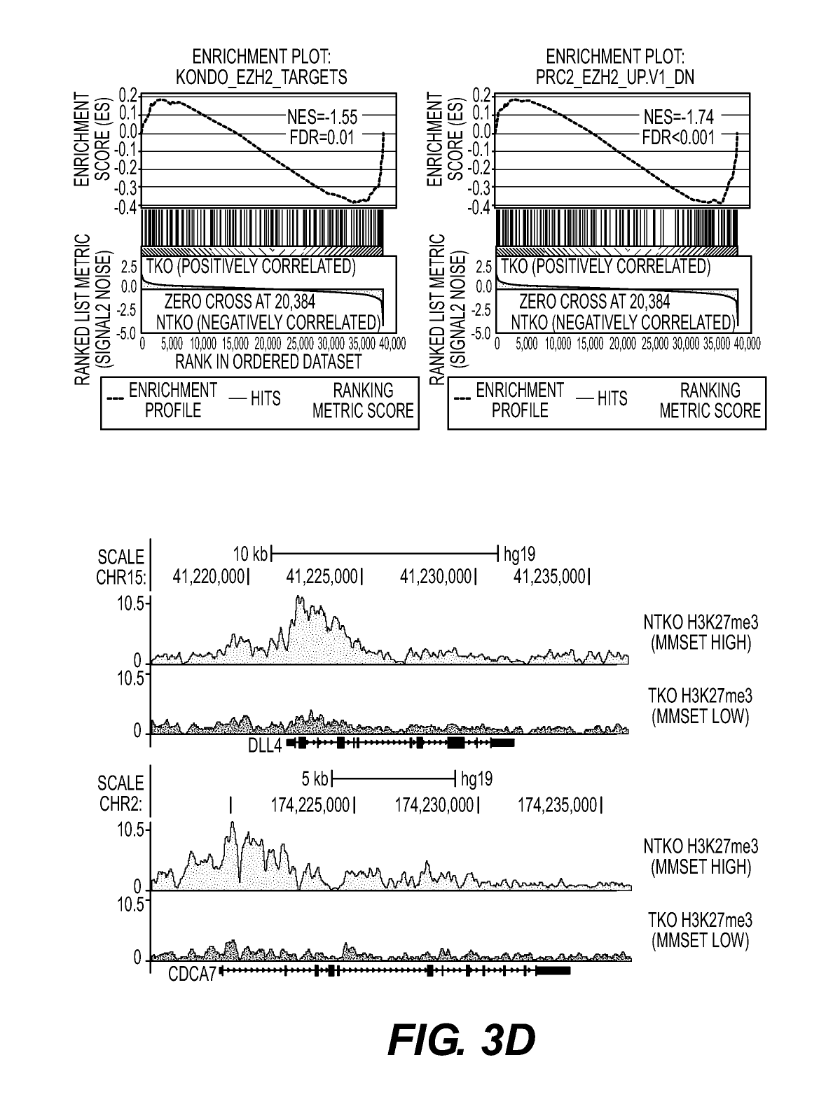

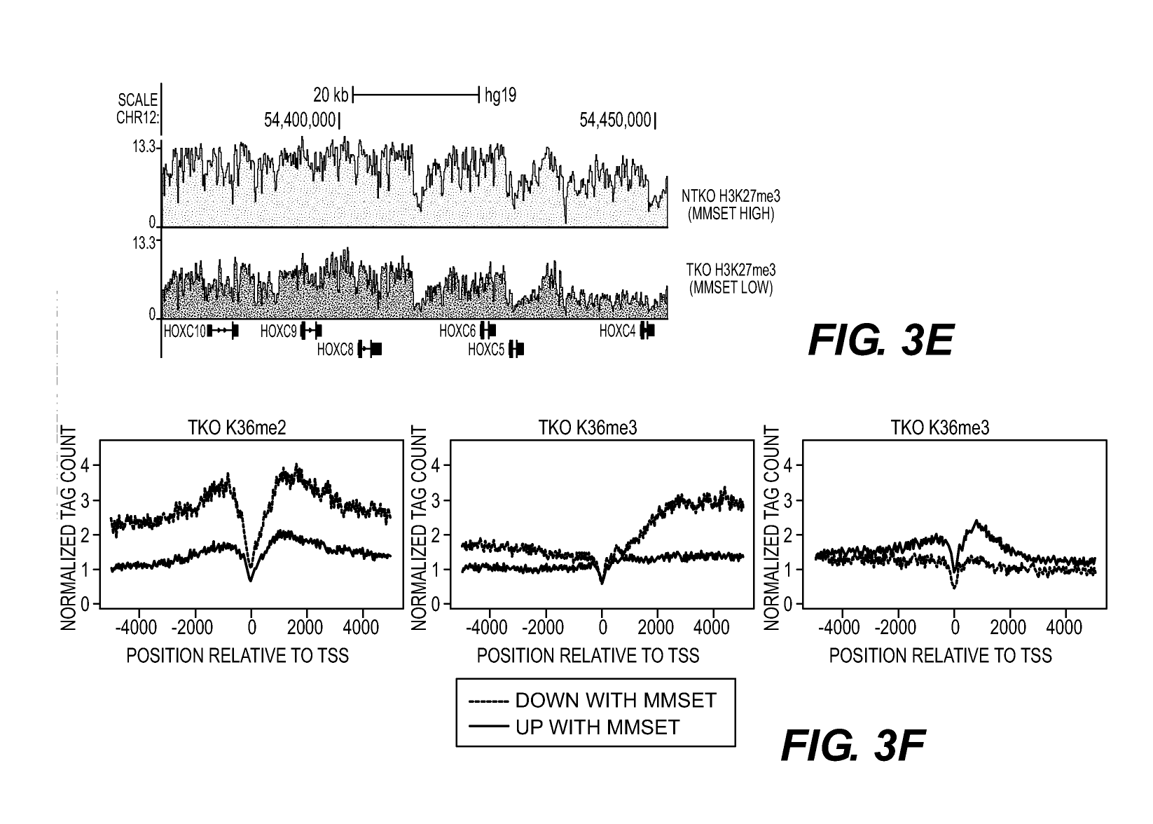

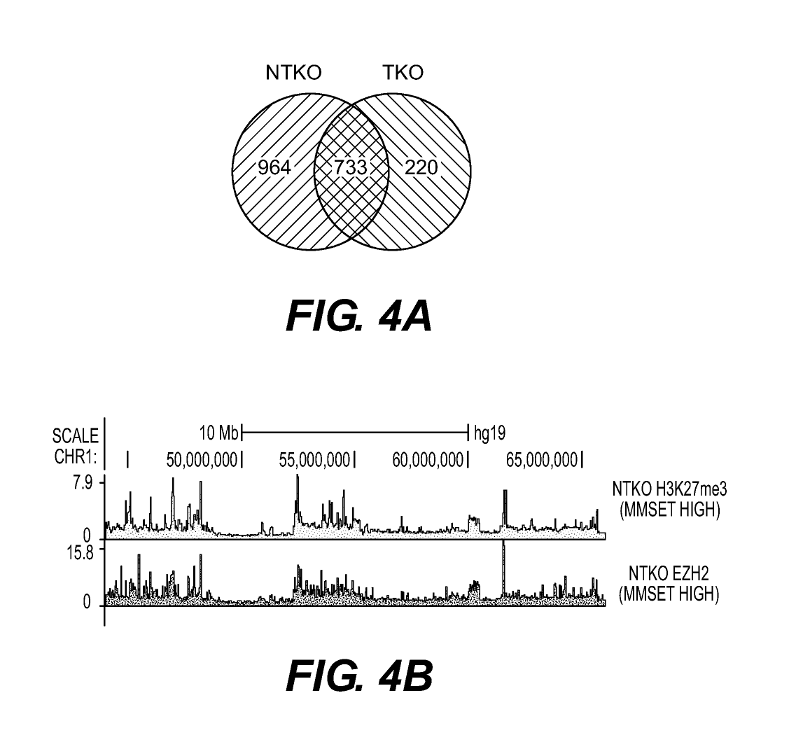

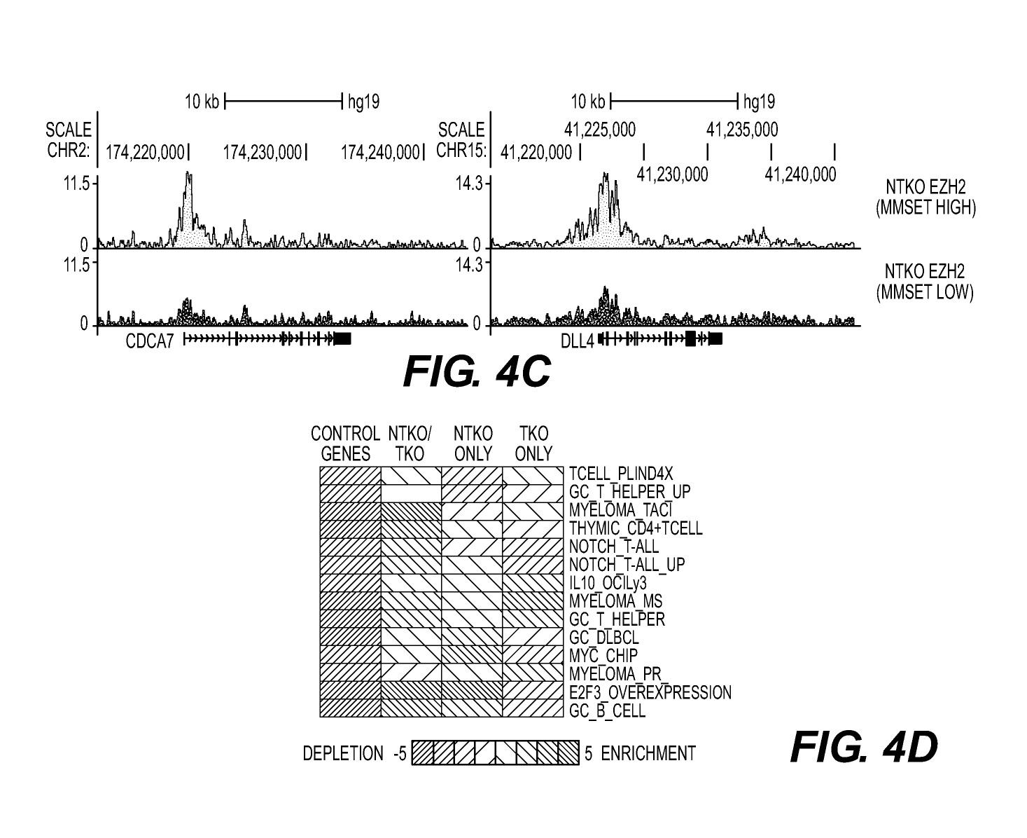

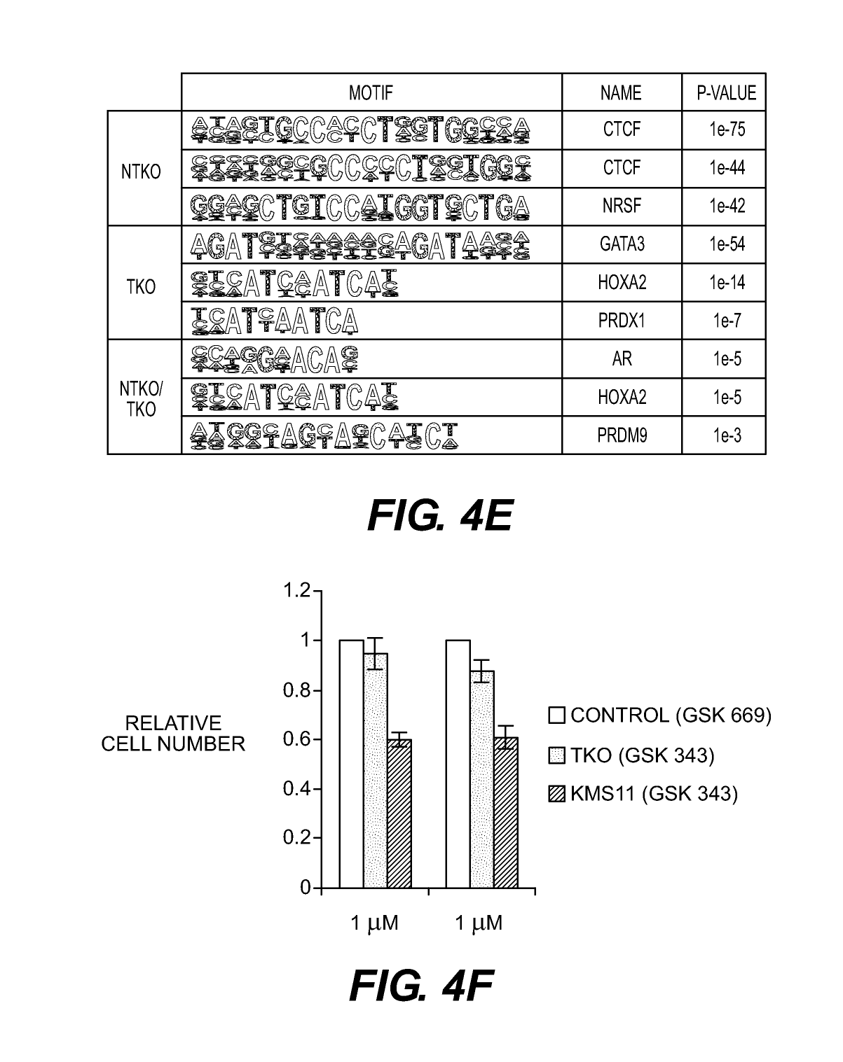

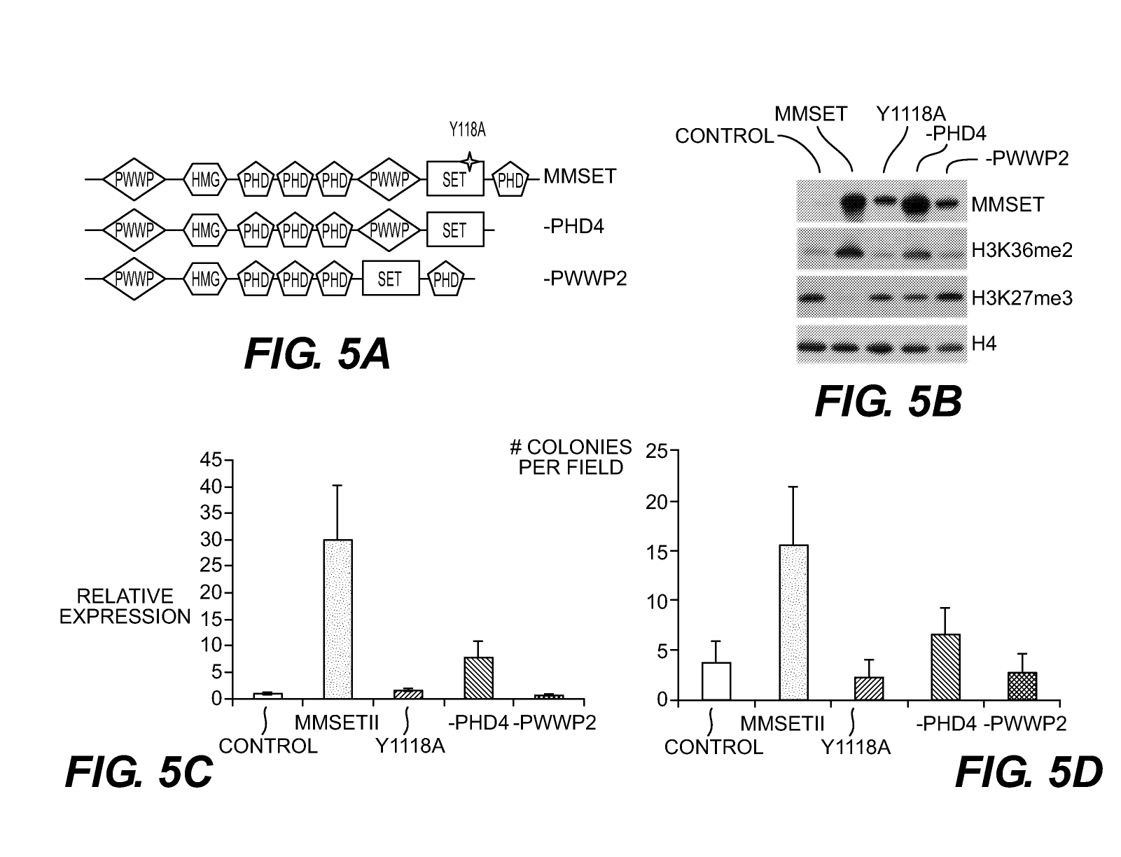

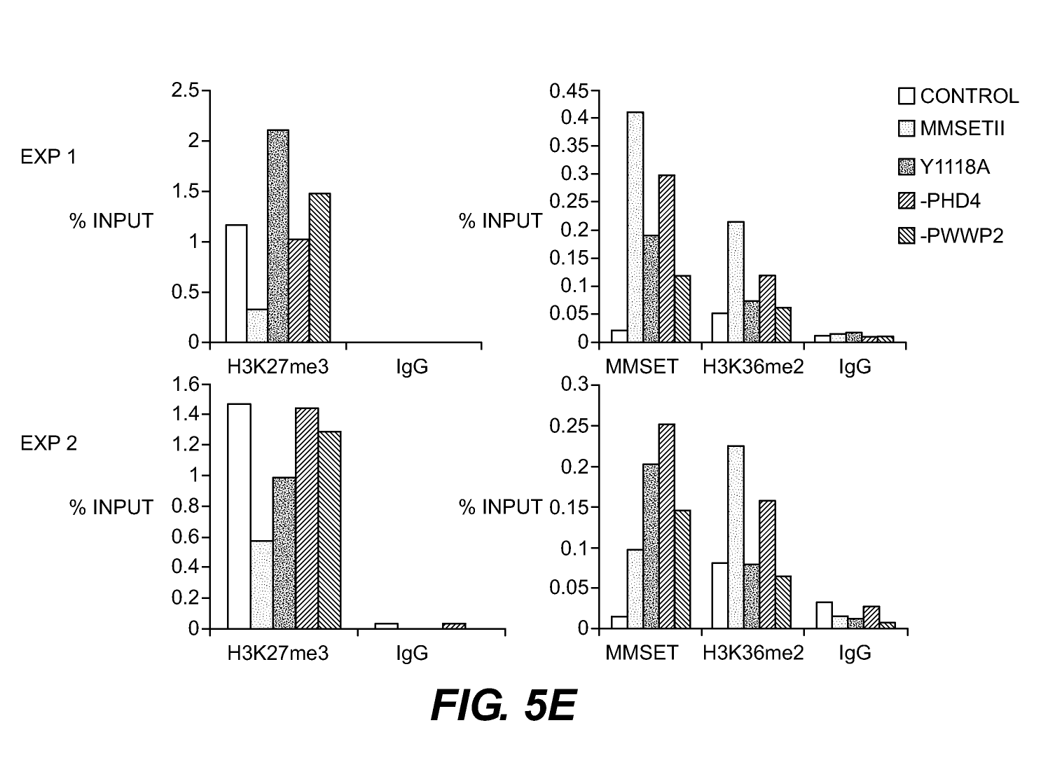

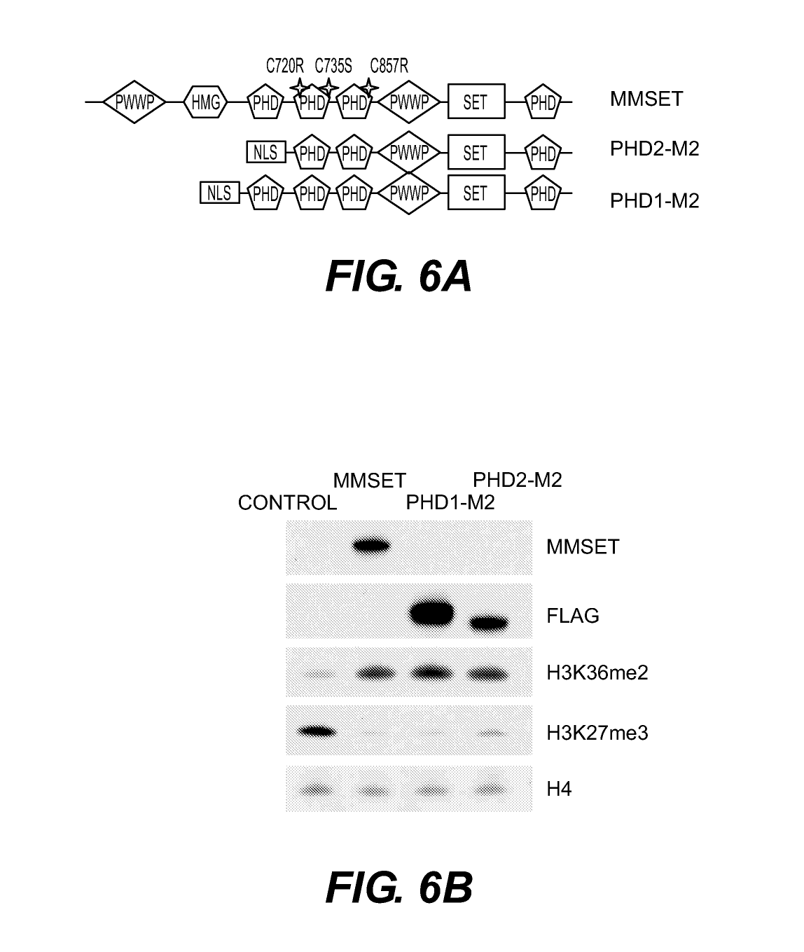

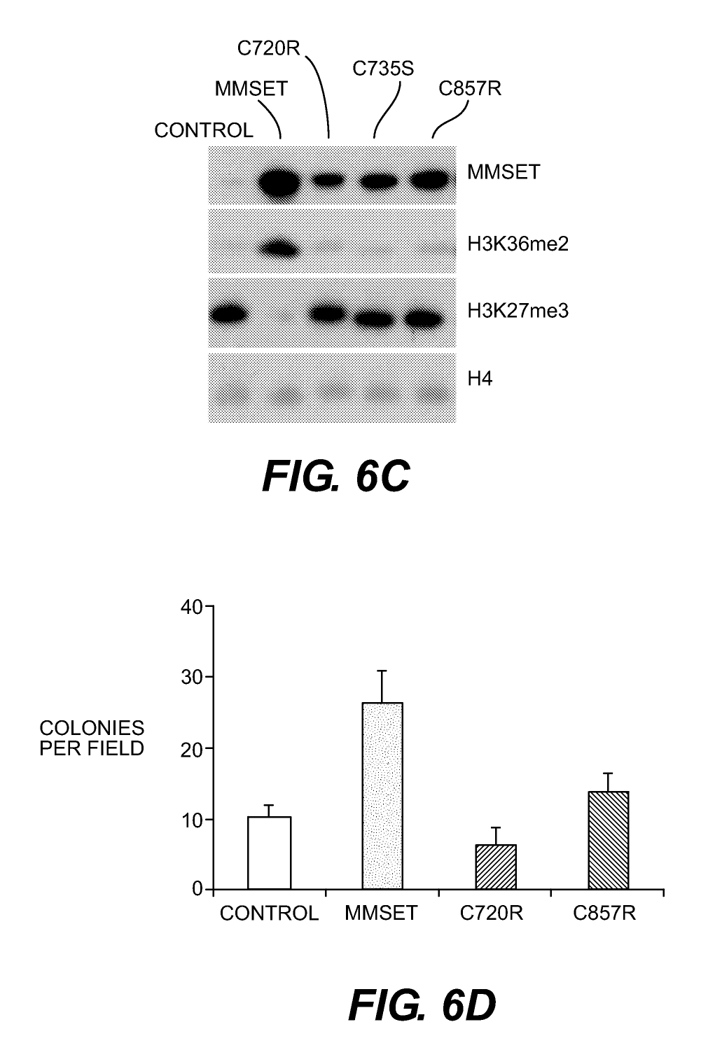

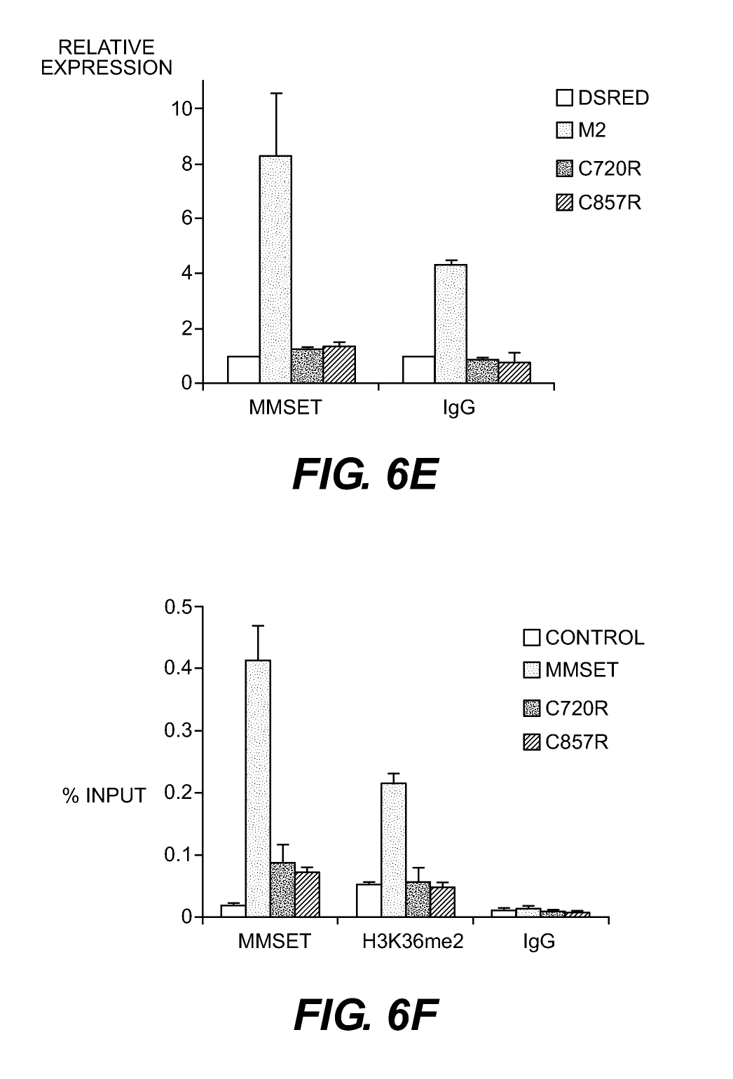

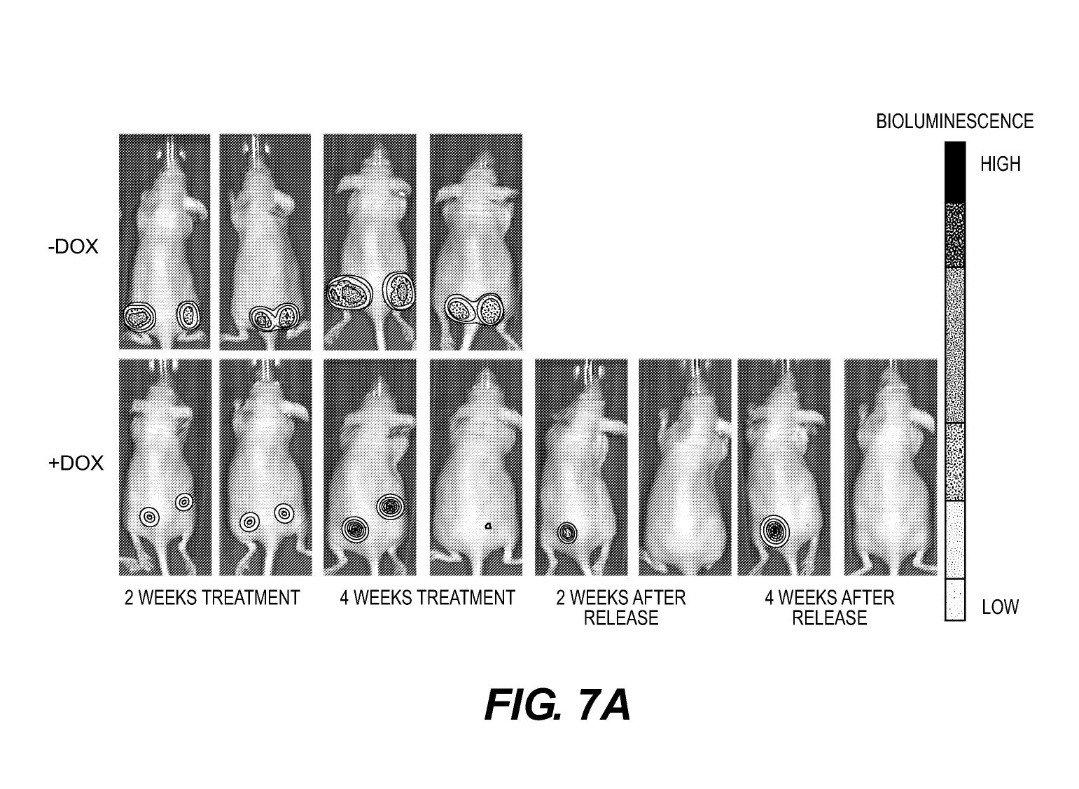

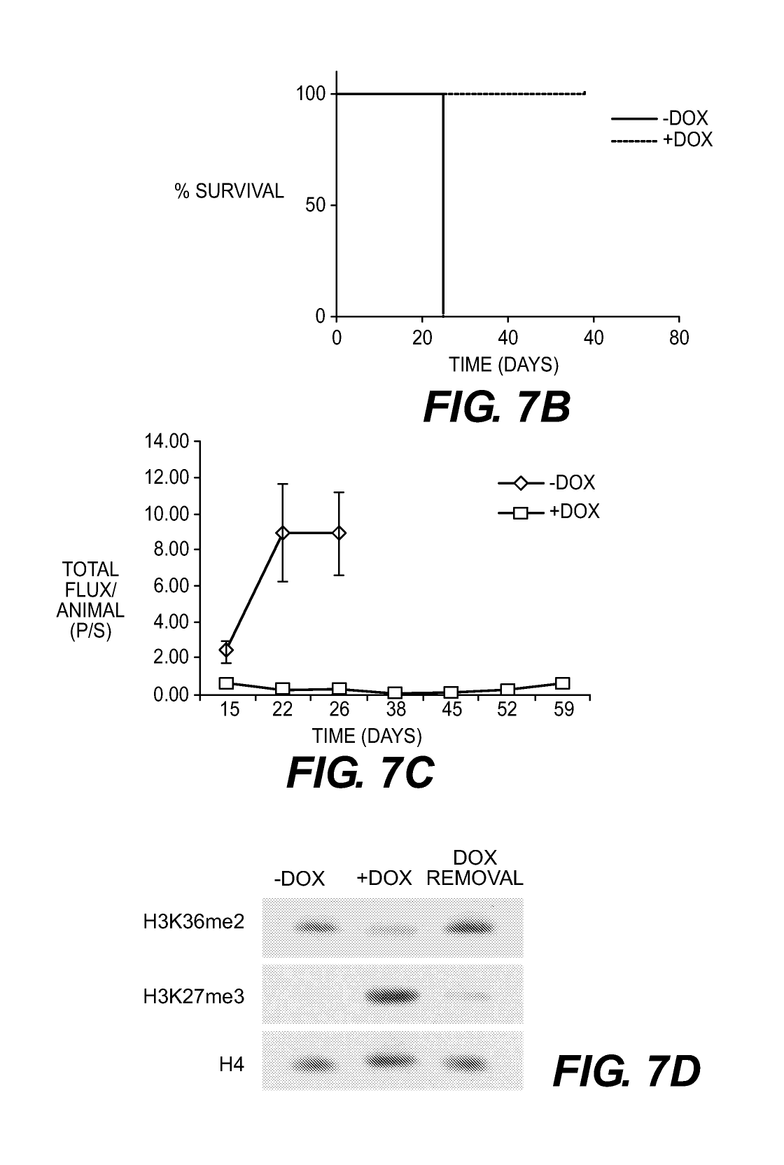

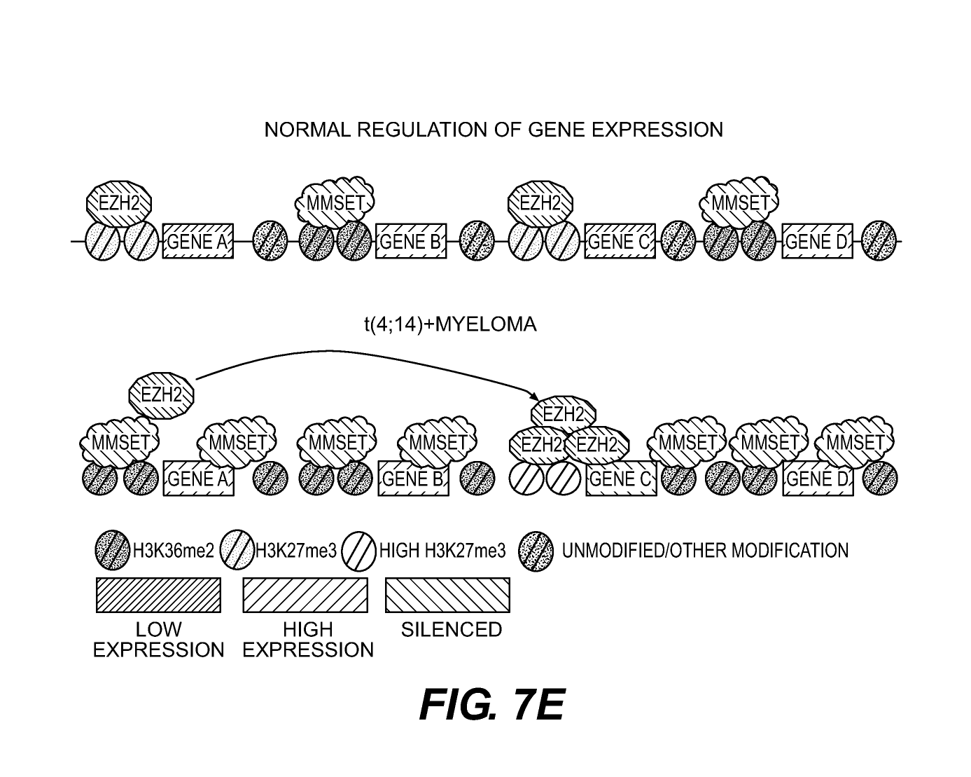

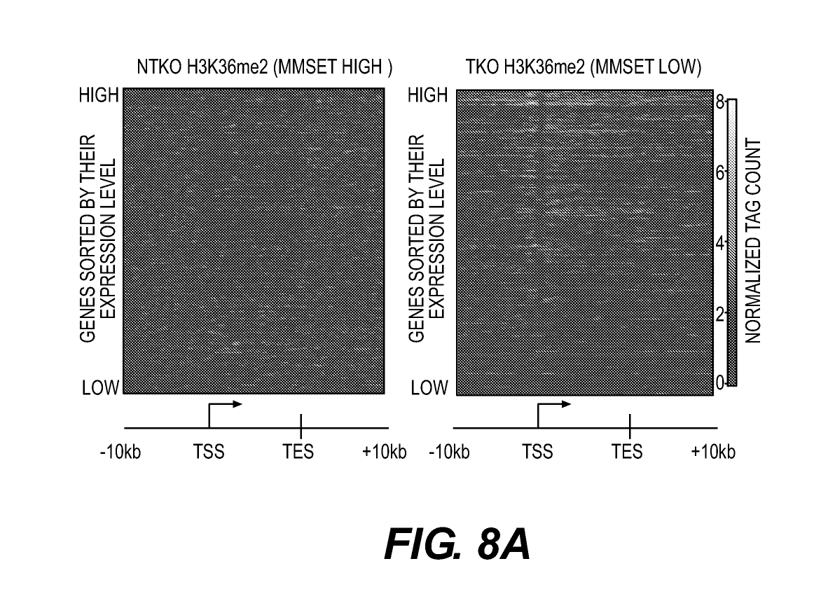

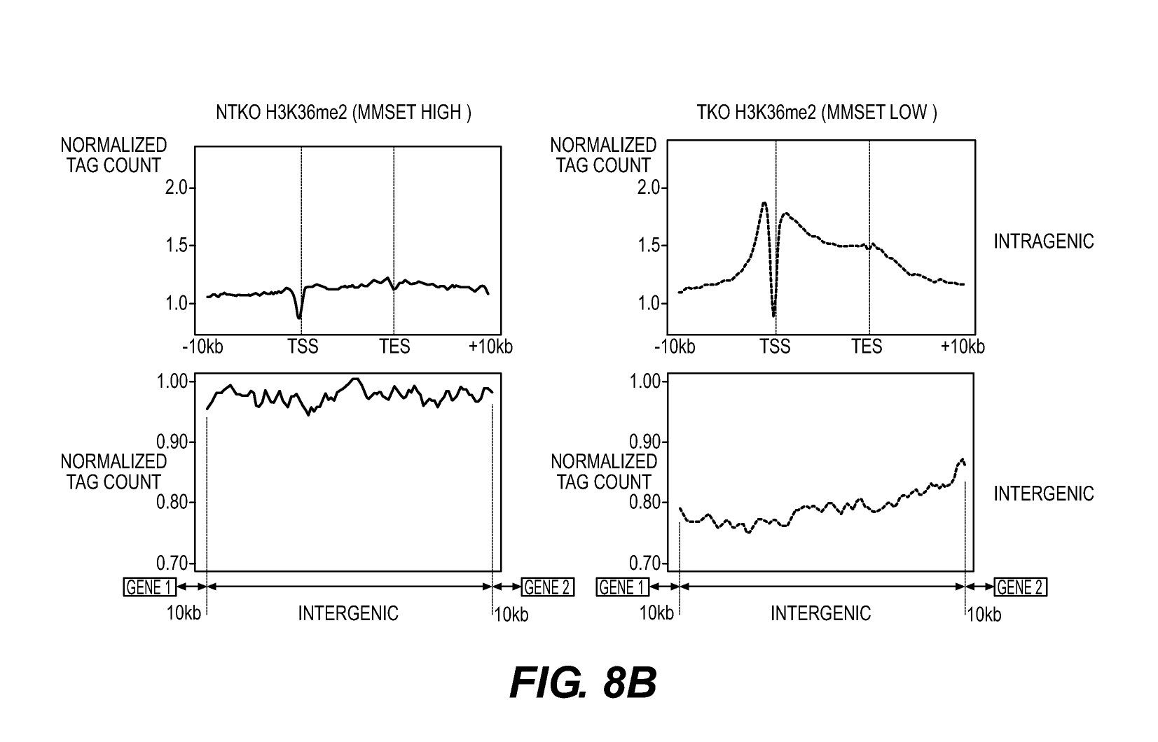

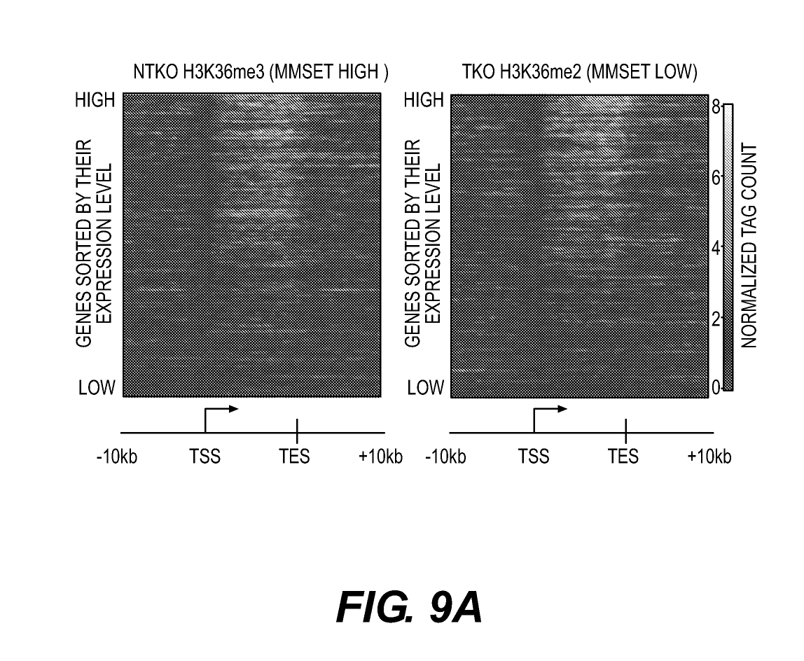

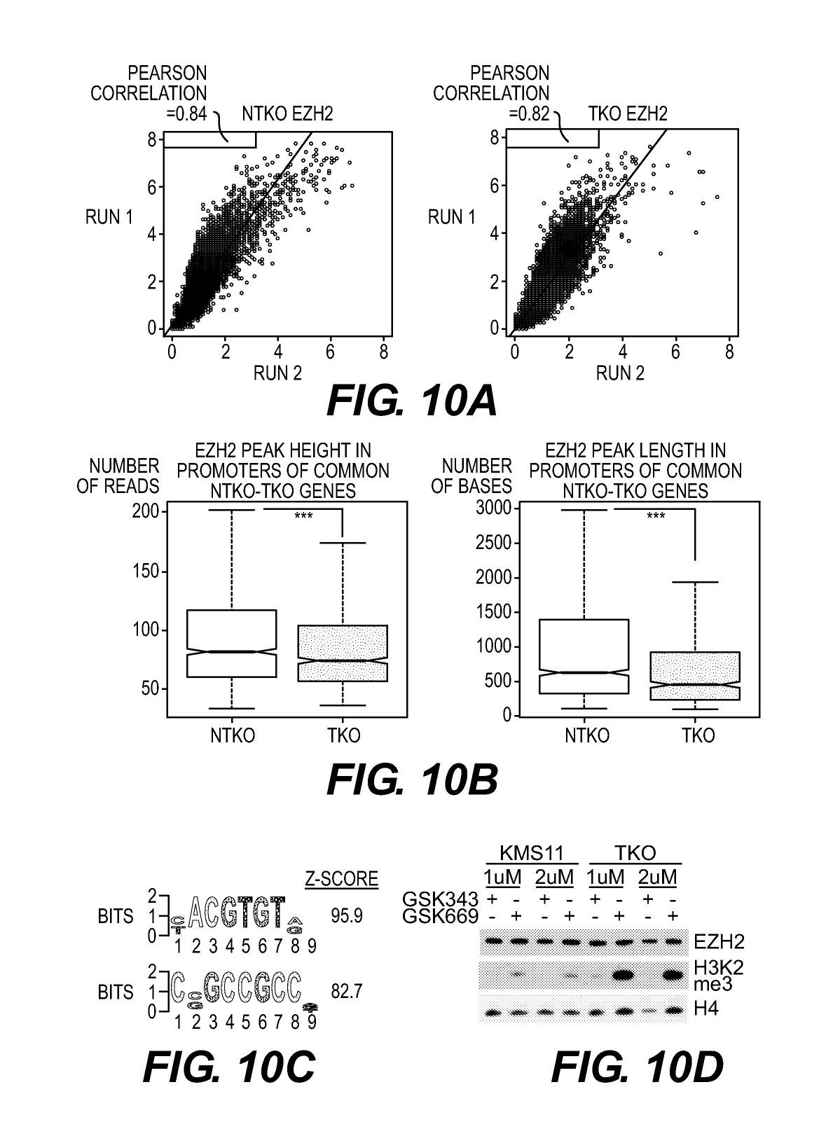

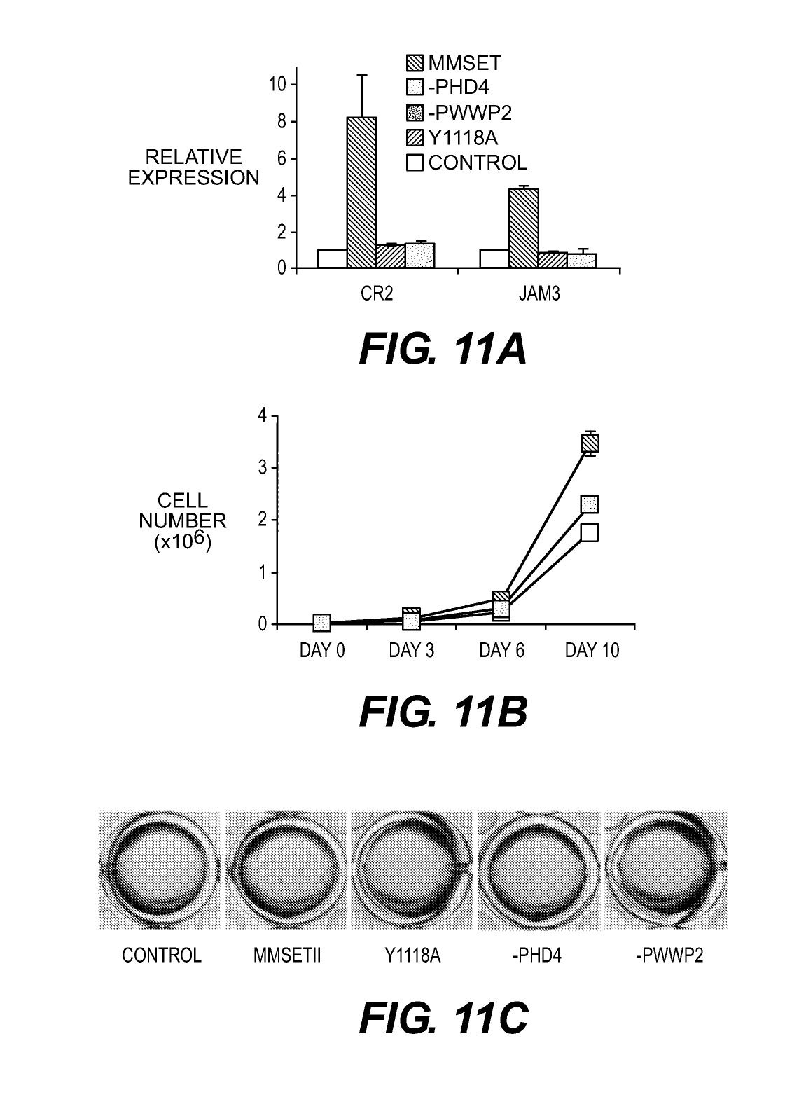

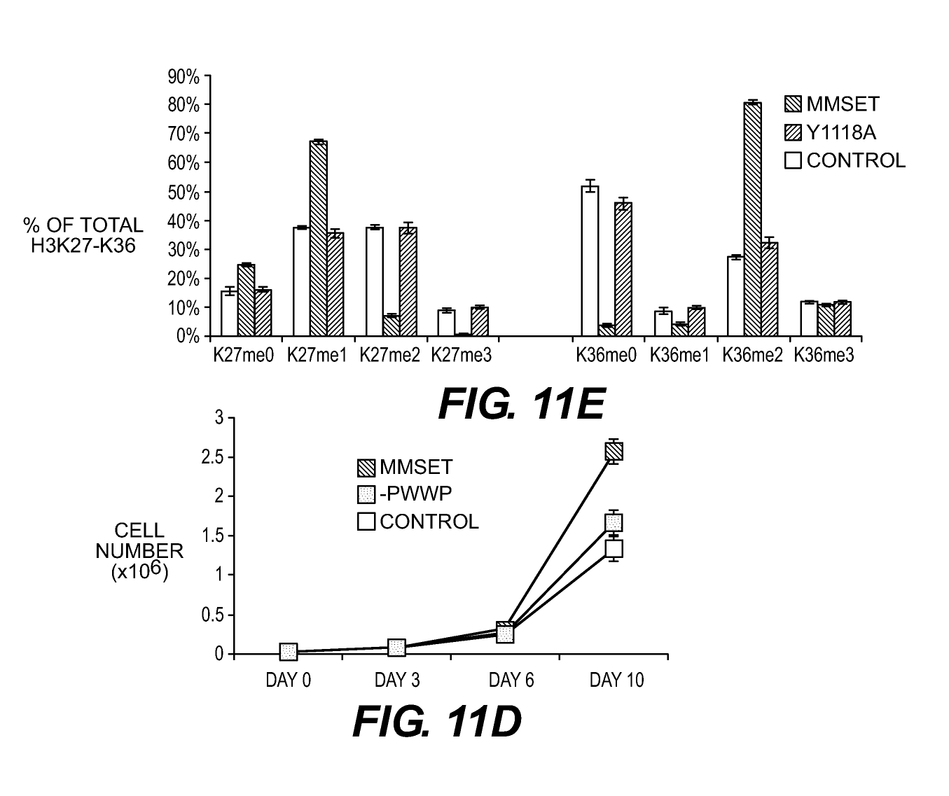

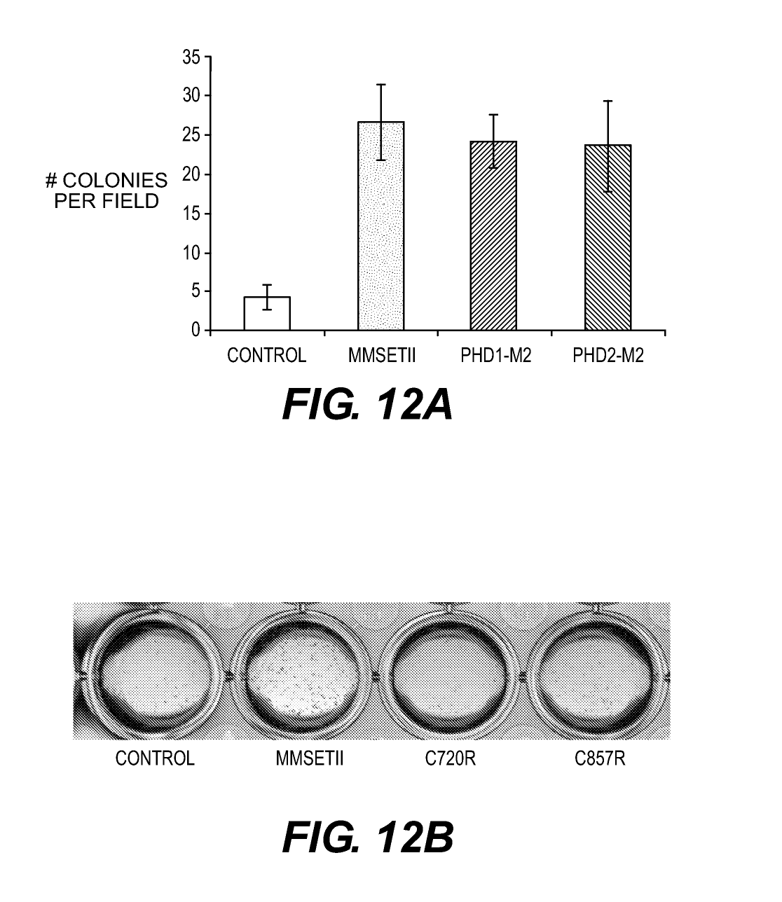

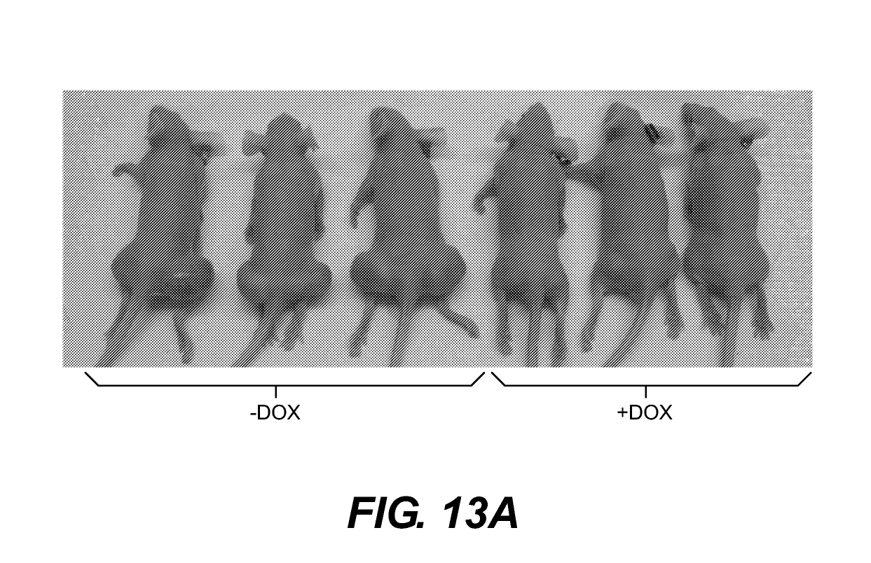

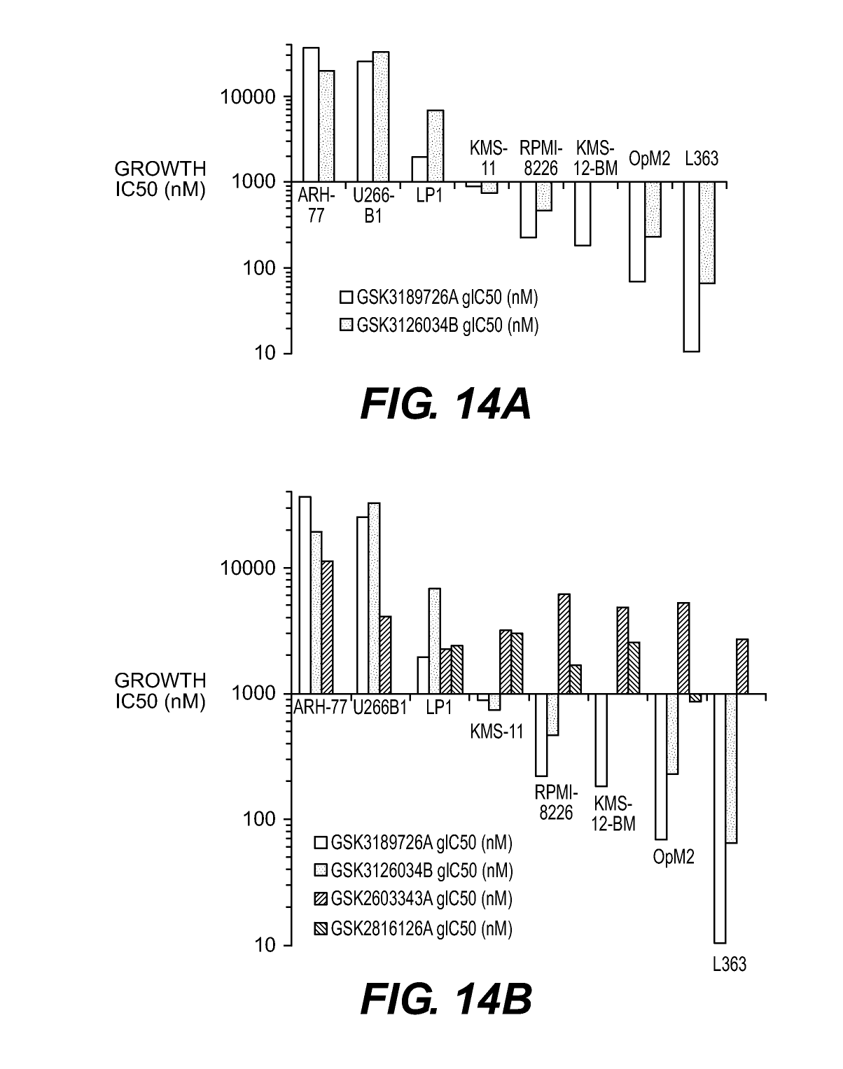

(A) Dot plot representing differentially expressed genes in NTKO and TKO cells from the microarray data. Genes upregulated and downregulated by MMSET (p<0.002) are in red (522 genes) and green (308 genes), respectively; genes that do not change expression are in dark blue (10,884 genes) and genes that are not expressed (2,234 genes) in either cell line are light blue. (B) Tag density profile of H3K36me2 distribution across different gene groups from A. Ratio between the number of reads in NTKO and TKO cells is presented on the y-axis. (C) Quantitative RT-PCR validation of several genes upregulated and downregulated by MMSET overexpression. (D) Heatmaps of H3K36me3 distribution between NTKO (left) and TKO (right) cells. Data were plotted as in FIG. 1B. (E) Density tracks for H3K36me2 and H3K36me3 on a gene upregulated by MMSET (CR2—upper panel) and a gene downregulated by MMSET overexpression (DLL4—lower panel). H3K36me2 tracks are in red and green for NTKO and TKO cells, respectively; H3K36me3 tracks are in blue (NTKO) and fuchsia (TKO).

FIG. 3. MMSET alters genome-wide patterns of H3K27me3 methylation.

(A) Tag density profile of H3K27me3 distribution across different gene groups from FIG. 2A. The ratio between read numbers in NTKO and TKO cells is presented on the y-axis. (B) UCSC genome browser display of H3K27me3 density tracks surrounding the transcription start site of two MMSET activated genes, CA2 (top) and CR2 (bottom). (C) GSEA analysis of genes upregulated by MMSET shows enrichment of previously identified EZH2 target genes. (D) UCSC genome browser display of H3K27me3 density tracks surrounding the transcription start site of two MMSET repressed genes, DLL4 (top) and CDCA7 (bottom). (E) UCSC genome browser of H3K27me3 enrichment on non-expressed genes of the HOXC cluster. (F) Tag density profile of H3K36me2 (left), H3K36me3 (middle) and H3K27me3 (right) distribution of differentially expressed genes in TKO cells.

FIG. 4. MMSET alters EZH2 binding in t(4;14)+ myeloma cells

(A) Venn diagram showing overlap of genes bound at their promoters by EZH2 in NTKO (blue) and TKO (yellow) cells. (B) UCSC genome browser display of H3K27me3 (top, gray) and EZH2 binding (bottom, red) in NTKO cells. (C) UCSC genome browser display of EZH2 ChIP-seq tracks in NTKO (top, red) and TKO (bottom, green) cells associated with MMSET-repressed genes, CDCA7 (left) and DLL4 (right). (D) Heat map of over-represented gene categories among genes bound by EZH2 in either NTKO cells, TKO cells or both cell types. Enrichment was measured using iPAGE analysis [67]. (E) Motif analysis using HOMER [66] identified conserved sequences bound by EZH2 in NTKO cells, TKO cells or both cell types. (F) Relative cell number of MMSET-high and MMSET-low cells treated with indicated doses of the EZH2 small molecule inhibitor (GSK343). Inactive compound, GSK669, was used as a control. Graph represents four independent experiments+/−standard deviation.

FIG. 5. The MMSET-induced epigenetic switch depends on multiple domains

(A) Diagram of the wild-type (amino acids 1-1365) and deletion MMSET constructs used for repletion experiments. Star represents location of the Y1118A mutation. −PHD construct encompasses amino acids 1-1209 and −PWWP2 construct is missing amino acids 877-995. (B) Representative immunoblot of nuclear extracts from repleted TKO cells was probed with the indicated antibodies. At least two independent infections were performed for each construct. (C) Representative qRT-PCR for JAM2 in repleted TKO cells. Measurements were performed from multiple infections in at least duplicate and the graph represents average expression+/−standard deviation. (D) Methylcellulose colony formation assay using repleted TKO cells. Experiment was performed in triplicate and at least six different fields were counted. Graph represents average colony count+/−standard deviation. (E) ChIP assay on the JAM2 promoter using repleted TKO cell. Two independent experiments are shown.

FIG. 6. PHD fingers 2 and 3 are required for proper MMSET function

(A) Diagram of the MMSET constructs used. PHD2-M2 and PHD1-M2 constructs start at amino acid 712 and 657, respectively. Mutations in the PHD domains are indicated as stars above the full-length MMSET. (B) Representative immunoblot on nuclear extracts from repleted TKO cells probed with the indicated antibodies. At least two independent infections were performed for each construct. (C) Nuclear extracts from the TKO cells repleted with PHD mutants were immunoblotted with the indicated antibodies. At least two independent infections were performed for each construct. (D) Colony formation with TKO cells repleted with mutated PHD constructs. Experiment was performed in triplicate and at least six different fields were counted. Graph represents average colony count+/−standard deviation. (E) Quantitative RT-PCR of MMSET target genes CR2 and JAM3 using RNA from repleted TKO cells. Experiment was performed in duplicate and graph represents average gene expression+/−standard deviation. (F) Mutations in the PHD fingers prevent MMSET binding to chromatin. ChIP assay on the promoter of JAM2. Experiment was performed in duplicate and graph represents average enrichment+/−standard deviation.

FIG. 7. Targeting MMSET in t(4;14)+ tumors prolongs survival

(A) Mouse xenograft model using t(4;14)+ KMS11 cells harboring a luciferase gene and a doxycycline-inducible MMSET-specific shRNA. Animals that were not administered doxycycline (−Dox) are shown on the top and animals that were given doxycycline are shown on the bottom (+Dox). Two representative animals are shown from each group (n=5) at the following timepoints: 2 weeks after treatment; 4 weeks after treatment; 2 weeks after release from Dox; and 4 weeks after release from Dox. −Dox animals were sacrificed 26 days after treatment initiation due to tumor size. The same two animals are shown at each time points. (B) Kaplan-Meier curve of the xenograft mouse experiment from A. (C) Measurement of the luciferin signal in treated (black) and untreated (red) mice over time. (D) Immunoblot for H3K36me2 and H3K27me3 using nuclear extracts from tumors isolated from the untreated or doxycycline-treated mice (E) Proposed model for explaining how MMSET overexpression alters the epigenetic landscape of myeloma cells. Methylation of H3K36 by MMSET induces a global decrease of H3K27me3, leading to activation of gene expression (bottom). Additionally, an overabundance of K36 methylation alters genome-wide EZH2 binding, inducing focal increases in H3K27 methylation and gene repression.

FIG. 8

(A) Heatmaps of H3K36me2 distribution between NTKO (left) and TKO (right) cells using replicate samples. Data were plotted as in FIG. 1B. (B) Average read density across 15,386 genes and 6,172 intergenic regions using replicate samples.

FIG. 9

Heatmaps of H3K36me3 distribution in replicate samples in NTKO (left) and TKO (right) cells. Data were plotted as in FIG. 1B.

FIG. 10

(A) Correlation plot of two independent EZH2 ChIP-seq experiments in NTKO (left) and TKO (right) cells. (B) Box plot representing average height (left) or average length (right) of EZH2 peaks common in both NTKO and TKO cells. Statistical significance was determined using Welch Two Sample t-test (***p<1e-6). (C) Motif analysis using FIRE [65] identified sequences enriched in EZH2-bound peaks in NTKO cells. (D) Immunoblot of nuclear extracts from KMS11 and TKO cells treated with 1 μM or 2 μM GSK343 for seven days. Inactive compound GSK669 was used as a control.

FIG. 11

(A) Quantitative RT-PCR for JAM3 and CR2 using RNA from the TKO repletion experiment. Experiment was performed in duplicate and graph represents average gene expression+/−standard deviation. (B) Growth curve of TKO cells repleted with either empty vector, wild-type MMSET or MMSET lacking PHD4. Experiment was performed in triplicate and graph represents average cell growth+/−standard deviation. (C) Images of the colony forming assay using repleted TKO cells. (D) Mass spectrometry analysis of H3K27 and H3K36 methylation from TKO cells expressing vector control, wild-type MMSET or enzymatically inactive Y1118A mutant MMSET. Analysis was performed as previously described [26]. (E) Growth curve of TKO cells infected with vector control, wild-type MMSET or MMSET lacking the second PWWP domain. Experiment was performed in triplicate and graph represents average cell growth+/−standard deviation.

FIG. 12

(A) Colony forming assay, using TKO cells repleted with vector control, wild-type MMSET, PHD1-M2 or PHD2-M2 constructs. Experiment was performed in triplicate and at least six different fields were counted. Graph represents average colony count+/−standard deviation. (B) Image of the colony assay using TKO cells repleted with vector control, wild-type MMSET or MMSET mutated at cysteines 720 or 857.

FIG. 13

Xenograft mice after four weeks of treatment. Three mice on the left were untreated while the three mice on the right received doxycycline in their drinking water.

FIG. 14

FIGS. 14A and B show the gIC50 results of various cell lines treated with specific EZH2 inhibitors.

### DETAILED DESCRIPTION OF THE INVENTION

Overexpression of the histone methyltransferase MMSET in t(4;14)+ multiple myeloma patients is believed to be the driving factor in the pathogenesis of this subtype of myeloma. MMSET catalyzes dimethylation of lysine 36 on histone H3 (H3K36me2), and its overexpression causes a global increase in H3K36me2, redistributing this mark in a broad, elevated level across the genome. Here, we demonstrate that an increased level of MMSET also induces a global reduction of lysine 27 trimethylation on histone H3 (H3K27me3). Despite the net decrease in H3K27 methylation, specific genomic loci exhibit enhanced recruitment of the EZH2 histone methyltransferase and become hypermethylated on this residue. These effects likely contribute to the myeloma phenotype since MMSET-overexpressing cells displayed increased sensitivity to EZH2 inhibition. Furthermore, we demonstrate that such MMSET-mediated epigenetic changes require a number of functional domains within the protein, including PHD domains that mediate MMSET recruitment to chromatin. In vivo, targeting of MMSET by an inducible shRNA reversed histone methylation changes and led to regression of established tumors in athymic mice. Together, our work elucidates previously unrecognized interplay between MMSET and EZH2 in myeloma oncogenesis and identifies domains to be considered when designing inhibitors of MMSET function.

Epigenetic control of gene expression plays a critical role in many biological processes and aberrant chromatin regulation is the driving factor in a multitude of diseases, including cancer. Through studies of chromosomal rearrangements, copy number changes, and more recently, sequencing of cancer genomes, it has become apparent that genetic alterations of enzymes responsible for covalent modification of histones or DNA, including histone methyltransferases (HMTs), are a recurrent theme in the pathogenesis of malignancy. Recently, HMTs have attracted particular interest due to their potential as therapeutic targets [1], but our understanding of the mechanisms by which abnormal histone methylation leads to disease development is still incomplete.

The specificity of each HMT is encoded within the catalytic SET (Suppressor of variegation, Enhancer of zeste and Trithorax) domain. For example, trimethylation of lysine 27 on histone H3 (H3K27me3) is mediated by the EZH2 protein, a member of the Polycomb Repressive Complex 2 (PRC2) [2]. Binding of EZH2 and the presence of the H3K27me3 mark are found at transcriptionally repressed loci and have been shown to play a role in recruitment of additional transcriptional repressors, including DNA methyltransferases (DNMTs) [3, 4]. EZH2 gain-of-function mutations that enhance H3K27me3 levels are pathogenic for germinal center type large B cell lymphomas [5, 6], whereas global loss of EZH2 function due to mutation/deletion of EZH2 or associated factors such as SUZ12, EED and ASXL1 are associated with myeloid neoplasms [7-9].
MMSET (WHSC1/NSD2) is a histone methyltransferase whose enzymatic specificity in vivo is towards dimethylation of lysine 36 on histone H3 (H3K36me2) [10-12], an epigenetic mark associated with transcriptionally active loci [13]. Heterozygous deletions of MMSET are implicated in the developmental disorder Wolf-Hirschhorn syndrome (WHS), characterized by cognitive and developmental defects [14]. Similar phenotypic defects are observed in MMSET-deficient mice [15]. Alterations in MMSET expression are also linked to cancer. This was first described in multiple myeloma (MM), where ˜20% of cases overexpress MMSET due to the translocation t(4;14) [16], which places the MMSET and FGFR3 loci under regulation of strong immunoglobulin enhancers, leading to abnormally high levels of these factors [17]. However, in 30% of cases, FGFR3 expression is not affected, suggesting that misregulation of MMSET may be the driving lesion of the disease [18, 19]. A growing body of literature demonstrates that increased expression of MMSET is associated with advanced stage solid tumors, including prostate, bladder, lung and skin cancers, where it may control oncogenic properties such as the epithelial-mesenchymal transition [20-23]. Furthermore, we recently identified a recurrent gain-of-function mutation of MMSET (E1099K) most commonly found in lymphoid malignancies, which enhances its methyltransferase activity and may functionally mimic overexpression seen in other cancers [24]. The epigenetic alterations and biological consequences of MMSET overexpression in cancer are beginning to be elucidated. We and others showed that downregulation of MMSET expression in t(4;14)+ cell lines leads to decreased proliferation and loss of clonogenic potential [12, 25]. In myeloma and prostate cells, overexpression of MMSET causes a dramatic global increase in H3K36me2, accompanied with a concomitant genome-wide loss of H3K27me3 [12, 20, 26]. The change in histone methylation is dependent on the HMT activity of MMSET and leads to altered chromatin structure and aberrant gene expression [12].
In this work, we aimed to elucidate the mechanisms by which MMSET alters gene expression in MM. Genome-wide chromatin analysis showed that MMSET overexpression led to a widespread increase in H3K36me2 across promoters, gene bodies and intergenic regions, and gene activation correlated with removal of the inhibitory H3K27me3 chromatin mark. Surprisingly, overexpression of MMSET induced transcriptional repression at specific loci that became highly enriched for EZH2 and H3K27me3. This increase was associated with augmented sensitivity to small molecule inhibitors targeting EZH2 methyltransferase activity. The ability of an epigenetic regulator to modify histones or DNA depends on its ability to target specific loci through direct interaction with chromatin, or through recruitment by other transcriptional cofactors. We identified the domains of MMSET that are required for its recruitment to chromatin and that are necessary for methylation of H3K36 and loss of H3K27 methylation in t(4;14)+ cells. Both of these functions are necessary for the oncogenic potential of MMSET. Lastly, we validated MMSET as a therapeutic target by showing that loss of MMSET expression in established t(4; 14)+ tumors led to a decrease in tumor burden and an increase in survival. Together, our results reveal an interplay between H3K36 and H3K27 methylation in t(4;14)+ myeloma and identify the domains of MMSET that could be targeted in efforts to improve outcomes of this currently incurable disease.
Another goal of the present work was to look at UTX loss and MMSET expression. As discussed above, normal EZH2 enzyme activity results in H3K27me3 methylation, which acts repressively. UTX, a histone demethylase removes the H3K27me3 methylation and activating gene expression. Inactive UTX enzyme (resulting from, e.g. UTX somatic mutations and deletions that cause loss of histone demethylase activity), i.e. so called UTX loss, are present in a subset of cancer cases, e.g. in about 10 percent of multiple myeloma patients. There is a need to better understand the effect of UTX loss in clinically relevant cancer cell lines and in patients, and identify clinically applicable treatments for cancer, e.g. multiple myeloma.
The present provides for methods of treating cancer in a human in need thereof, comprising determining at least one of the following in a sample from said human: the presence an increased level of MMSET as compared to a control; or the presence or absence of a decreased level of a functional UTX protein as compared to a control; and
and administering to said human an effective amount of an EZH2 inhibitor or pharmaceutically acceptable salt thereof if the t(4,14) translocation in MMSET is present, or there is a decreased level of a functional UTX protein as compared to a control, or both.
The present invention also provides methods for treating cancer in a mammal, e.g. a human in need thereof, comprising determining at least one of the following in a sample from said mammal, e.g. human:
a. the presence or absence of an elevated level of MMSET expression as compared to a control; or
b. the presence or absence of an increased level of global H3K36me3 as compared to a control; or
c. the presence or absence of an decreased level of global H3K27me3 as compared to a control, or
d. the presence or absence of an increased level of global H3K27me2 as compared to a control; or
e. the presence or absence of a decreased level of global H3K27 methylation as compared to a control; or
f. the presence or absence of an increased level of H3K27me3 in gene targets of EZH2 and H3K27, e.g. gene loci methylated by EZH2 and H3K27, or in the HOXC cluster, or in the DLLL promoter, or in the CDCA7 promoter, as compared to a control; or
g. the presence or absence of an increased level of FGFR3 expression as compared to a control; or
   and administering to said human an effective amount of an EZH2 inhibitor or pharmaceutically acceptable salt thereof or an MMSET inhibitor or pharmaceutically acceptable salt thereof, or both, if one or more of the following are determined:
the presence or of an elevated level of MMSET expression; or
the presence an increased level of global H3K36me3 as; or
the presence of a decreased level of global H3K27me3; or
the presence of an increased level of global H3K27me2; or
the presence of a decreased level of global H3K27 methylation; or
the presence or absence of an increased H3K27me3 in gene targets of EZH2 and H3K27, e.g. gene loci methylated by EZH2 and H3K27, or in the HOXC cluster, or in the DLLL promoter, or in the CDCA7 promoter; or
the presence of an increased level of FGFR3 expression.
   The invention herein further provides method of treating cancer, e.g. a lymphoid malignancy, in a human in need thereof, comprising determining the presence or absence of an E1099 mutation in the MMSET protein, e.g. E1099K, in a sample from said human and administering an EZH2 inhibitor if it is determined said E1099 mutation, e.g. E1099K, is present in said sample.
   The determinations of a-g above, as well as any further determinations, e.g. of mutations described herein, may be done in any order.
   In further embodiments of the methods herein, other EZH2 mutations known in the art, e.g. Y641 or A677 or A687 are determined, either alone or in combination with one or more of the following, in any order: determining the presence or absence of an A687 mutation, e.g. A687V, or the presence or absence of an increased level of H3K27me2, or the presence or absence of an increased level of H3K27me3. In further embodiments the Y641 mutations are selected from the group consisting of Y641F, Y641S, Y641H, Y641N, or Y641C. In other further embodiments, the A677 mutation is A677G. In other further embodiments, the A687 mutation is A687V.
   Methods of detecting a mutation in EZH2, e.g. at E1099, are well known to one of skill in the art and are described herein in the detailed description and Examples. Methods of determining an increased level of methylation, e.g, H3K27me2 or H3K27Me3, H3K36me3 and global levels of H3K27 and H3K36 methylation relative to a control are well known in the art and shown in the Examples, and include, e.g., using an antibody specific for di or trimethylated lysine 27 of Histone 3. A control can be any one of skill in the art would choose, such as a matched cell from a human, a matched tissue from a human, a cell of the same origin as the tumor but known to have wild type EZH2, or a devised control that correlates with what is seen in non-cancerous cells of the same origin or in cells with wild-type EZH2.
   In other embodiments of the invention, the sample comprises at least one cancer cell. In certain such embodiments, the sample is a biological sample.
   In any one of the embodiments of the invention herein, the cancer is a myeloid dysplasia. In further embodiments, the cancer is agnogenic myeloid metaplasia. In other embodiments, the cancer is a myeloid malignancy cancer. In other embodiments, the cancer is a myeloma. In further embodiments of the method of the invention, the cancer is multiple myeloma. In further embodiments, the multiple myeloma is t(4;14)+ multiple myeloma.

In other embodiments, the cancer is lymphoma. In other embodiments, the cancer is a solid tumor. In further embodiments, the solid tumor cancer is selected from the group consisting of prostate cancer, bladder cancer, lung cancer, and skin cancer.

In any embodiment, the sample from a human comprises at least one cancer cell. One of skill in the art can ascertain what sample is appropriate, e.g. a blood sample for a blood cancer such as lymphoma or myeloma and a biopsy for a solid tumor, etc.

In certain embodiments, e.g. in those describing the detection of E1099 mutations, the cancer is a lymphoid malignancy, e.g. lymphoma. In further embodiments the lymphoma is selected from the group consisting of: B-cell acute lymphoblastic leukemia (B-cell ALL), germinal center B-cell (GCB), Diffuse Large B-cell Lymphoma (DLBCL), Splenic marginal zone lymphoma (SMZL), Waldenström's macroglobulinemia lymphoplasmacytic lymphoma (WM), Follicular lymphoma (FL), Mantle Cell Lymphoma (MCL), and Extra nodal marginal zone B-cell lymphoma of mucosa associated lymphoid tissue (MALT).

Disclosed herein a pharmaceutical compositions comprising an EZH2 inhibitor for use in treatment of cancer, wherein the cancer is characterized as having one or both of the following: the presence of increased level in MMSET expression as compared to a control; or

the presence of a decreased level of a functional UTX protein as compared to a control. In a further embodiment, the EZH2 inhibitor is one of Formula I, II, III, IV, or Compound B, or Compound C, or any other specific EZH2 inhibitor disclosed herein. In further embodiments, the cancer is a multiple myeloma described herein or a lymphoma described herein

Disclosed herein a pharmaceutical compositions comprising an EZH2 inhibitor for use in treatment of cancer, wherein the cancer is characterized as having one or more of the following:
the presence or of an elevated level of MMSET expression; or
the presence an increased level of global H3K36me3 as; or
the presence of a decreased level of global H3K27me3; or
the presence of an increased level of global H3K27me2; or
the presence of a decreased level of global H3K27 methylation; or
the presence or absence of an increased H3K27me3 in gene targets of EZH2 and H3K27, e.g. gene loci methylated by EZH2 and H3K27, or in the HOXC cluster, or in the DLLL promoter, or in the CDCA7 promoter; or
the presence of an increased level of FGFR3 expression.

### DEFINITIONS

The term “wild type” as is understood in the art refers to a polypeptide or polynucleotide sequence that occurs in a native population without genetic modification. As is also understood in the art, a “variant” includes a polypeptide or polynucleotide sequence having at least one modification to an amino acid or nucleic acid compared to the corresponding amino acid or nucleic acid found in a wild type polypeptide or polynucleotide, respectively. Included in the term variant is Single Nucleotide Polymorphism (SNP) where a single base pair distinction exists in the sequence of a nucleic acid strand compared to the most prevalently found (wild type) nucleic acid strand.

As used herein “genetic modification” or “genetically modified” or grammatical variations thereof refers to, but is not limited to, any suppression, substitution, amplification, deletion and/or insertion of one or more bases into DNA sequence(s). Also, as used herein “genetically modified” can refer to a gene encoding a polypeptide or a polypeptide having at least one deletion, substitution or suppression of a nucleic acid or amino acid, respectively. Genetic variants and/or SNPs can be identified by known methods. For example, wild type or SNPs can be identified by DNA amplification and sequencing techniques, DNA and RNA detection techniques, including, but not limited to Northern and Southern blot, respectively, and/or various biochip and array technologies. WT and mutant polypeptides can be detected by a variety of techniques including, but not limited to immunodiagnostic techniques such as ELISA and western Blot. As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

As is well known genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, “genotyping” a subject (or DNA or other sample) for a polymorphic allele of a gene(s) or a mutation in at least one polypeptide or gene encoding at least one polypeptide means detecting which mutated, allelic or polymorphic form(s) of the gene(s) or gene expression products (e.g., hnRNA, mRNA or protein) are present or absent in a subject (or a sample). Related RNA or protein expressed from such gene may also be used to detect mutant or polymorphic variation. As is well known in the art, an individual may be heterozygous or homozygous for a particular allele. More than two allelic forms may exist, thus there may be more than three possible genotypes. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g., sequencing, e.g., next generation sequencing (NGS)) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype or a tumor having at least one somatic mutation. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria.

As used herein, a human that is in need of treatment for cancer, may be “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

A human that is in need of treatment for cancer may alternatively have a tumor or cancer cells with somatic mutations, and genotyping or other detection of the mutations can be performs.

As used herein “response” to treatment and grammatical variations thereof, includes but is not limited to an improved clinical condition of a patient after the patient received medication. Response can also mean that a patient's condition does not worsen upon that start of treatment. Response can be defined by the measurement of certain manifestations of a disease or disorder. With respect to cancer, response can mean, but is not limited to, a reduction of the size and or number of tumors and/or tumor cells in a patient. Response can also be defined by other endpoints such as a reduction or attenuation in the number of pre-tumorous cells in a patient.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

Samples, e.g. biological samples, for testing or determining of one or more mutations may be selected from the group of proteins, nucleotides, cellular blebs or components, serum, cells, blood, blood components, urine and saliva. Testing for mutations may be conducted by several techniques known in the art and/or described herein.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

A “mutation” in a polypeptide or a gene encoding a polypeptide and grammatical variations thereof means a polypeptide or gene encoding a polypeptide having one or more allelic variants, splice variants, derivative variants, substitution variants, deletion variants, and/or insertion variants, fusion polypeptides, orthologs, and/or interspecies homologs. By way of example, at least one mutation of EZH2 would include an EZH2 in which part of all of the sequence of a polypeptide or gene encoding the polypeptide is absent or not expressed in the cell for at least one of the EZH2 proteins produced in the cell. For example, an EZH2 protein may be produced by a cell in a truncated form and the sequence of the truncated form may be wild type over the sequence of the truncate. A deletion may mean the absence of all or part of a gene or protein encoded by a gene. An EZH2 mutation also means a mutation at a single base in a polynucleotide, or a single amino acid substitution. Additionally, some of a protein expressed in or encoded by a cell may be mutated, e.g., at a single amino acid, while other copies of the same protein produced in the same cell may be wild type.

Mutations may be detected in the polynucleotide or translated protein by a number of methods well known in the art. These methods include, but are not limited to, sequencing, RT-PCR, and in situ hybridization, such as fluorescence-based in situ hybridization (FISH), antibody detection, protein degradation sequencing, etc. Epigenetic changes, such as methylation states, may also result in mutations and/or lack of expression of part or all of a protein from the corresponding polynucleotide encoding it.

As used herein “genetic abnormality” is meant a deletion, substitution, addition, translocation, amplification and the like relative to the normal native nucleic acid content of a cell of a subject. As used herein “gene encoding an EZH2 protein” means any part of a gene or polynucleotide encoding any EZH2 protein. Included within the meaning of this term are exons encoding EZH2. Gene encoding EZH2 proteins include but are not limited to genes encoding part or all of EZH2.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

As used herein “overexpressed” and “overexpression” and grammatical variations thereof means that a given cell produces an increased number of a certain protein relative to a normal cell. For instance, some tumor cells are known to overexpress Her2 or Erb2 on the cell surface compared with cells from normal breast tissue. Gene transfer experiments have shown that overexpression of HER2 will transform NIH 3T3 cells and also cause an increase in resistance to the toxic macrophage cytokine tumor necrosis factor. Hudziak et al., “Amplified Expression of the HER2/ERBB2 Oncogene Induces Resistance to Tumor Necrosis Factor Alpha in NIH 3T3 Cells”, Proc. Natl. Acad. Sci. USA 85, 5102-5106 (1988). Expression levels of a polypeptide in a particular cell can be effected by, but not limited to, mutations, deletions and/or substitutions of various regulatory elements and/or non-encoding sequence in the cell genome.

As used herein, “treatment” means any manner in which one or more symptoms associated with the disorder are beneficially altered. Accordingly, the term includes healing or amelioration of a symptom or side effect of the disorder or a decrease in the rate of advancement of the disorder.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be hematopoietic tumor, for example, tumors of blood cells or the like. Specific examples of clinical conditions based on such a tumor include leukemia such as chronic myelocytic leukemia or acute myelocytic leukemia; myeloma such as multiple myeloma; lymphoma and the like.

The cancer may be any cancer in which an abnormal number of blast cells are present or that is diagnosed as a haematological cancer or dysplasia, such as leukemia, myeloid malignancy or myeloid dysplasia, including but not limited to, undifferentiated acute myelogenous leukemia, myeloblastic leukemia, myeloblastic leukemia, promyelocytic leukemia, myelomonocytic leukemia, monocytic leukemia, erythroleukemia and megakaryoblastic leukemia. In one aspect, the cancer is a myeloid malignancy cancer. In another aspect, the cancer is leukemia. The leukemia may be acute lymphocytic leukemia, acute non-lymphocytic leukemia, acute myeloid leukemia (AML), chronic lymphocytic leukemia, chronic myelogenous (or myeloid) leukemia (CML), and chronic myelomonocytic leukemia (CMML). In one embodiment, the human has agnogenic myeloid metaplasia and/or poor-risk myelodysplasia (MDS). In some aspects the cancer is relapsed or refractory.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In some embodiments, the sample is selected from the group consisting of cancer cells, tumor cells, cells, blood, blood components, urine and saliva.

### Compounds of the Invention

In certain embodiments of the methods of treating cancer in a human in need thereof, the EZH2 inhibitor is of Formula I:

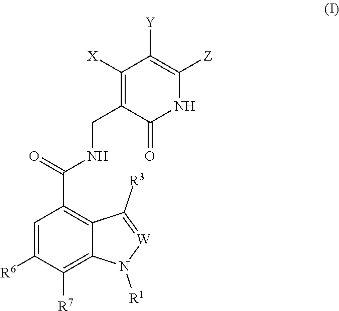

wherein:

W is N or CR²;

X and Z are each independently selected from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₉)alkyl or —(C₂-C₈)alkenyl, unsubstituted or substituted heteroaryl, unsubstituted or substituted heteroaryl-(C₁-C₈)alkyl or —(C₂-C₈)alkenyl, halogen, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};

Y is hydrogen or halogen;

R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, or —CONR^{a}NR^{a}R^{b};

When present R² is hydrogen, (C₁-C₈)alkyl, trifluoromethyl, alkoxy, or halogen, in which said (C₁-C₈)alkyl may be substituted with one to two groups selected from amino and (C₁-C₃)alkylamino;

R⁷ is hydrogen, (C₁-C₃)alkyl, or alkoxy; R³ is hydrogen, (C₁-C₈)alkyl, cyano, trifluoromethyl, —NR^{a}R^{b}, or halogen;

R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};

each R^{c} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};

R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2, or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

or a pharmaceutically acceptable salt thereof.

Compounds of Formula I, and methods of making the same are disclosed in WO2011/140324, which is incorporated by reference in its entirety herein.

In a further embodiment, EZH2 inhibitor is a compound of Formula (I) wherein W is CR²′ or a pharmaceutically acceptable salt thereof.

In yet a further embodiment, the EZH2 inhibitor is a Compound of Formula I having Formula B:

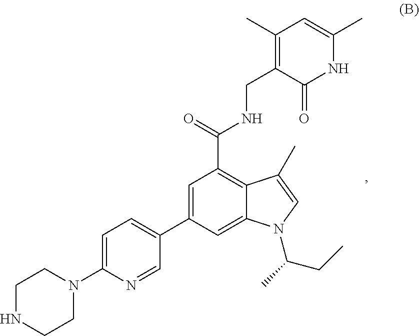 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt thereof.

Compounds having Formula B and methods of making the same are disclosed in WO 2011/140324, e.g. Example 270.

In another embodiment, the EZH2 inhibitor is Compound A having formula 1-(1-methylethyl)-N-[(6-methyl-2-oxo-4-propyl-1,2-dihydro-3-pyridinyl)methyl]-6-[6-(4-methyl-1-piperazinyl)-3-pyridinyl]-1H-indazole-4-carboxamide;

Additional EZH2 inhibitors are well known in the art. For example, EZH2 inhibitors are disclosed in WO 2011/140324, WO 2011/140325 and WO 2012/075080, each of which is incorporated by reference herein in its entirety. In any of the embodiments herein, the EZH2 inhibitor may be a compound disclosed in WO 2011/140324, WO 2011/140325 or WO 2012/075080.

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁C₈alkyl” refers to an alkyl group having at least 1 and up to 8 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, and n-octyl and branched analogs of the latter 5 normal alkanes.

The term “alkoxy” as used herein means —O(C₁C₈alkyl) including —OCH₃, —OCH₂CH₃ and —OC(CH₃)₃ and the like per the definition of alkyl above.

The term “alkylthio” as used herein is meant —S(C₁C₈alkyl) including —SCH₃, —SCH₂CH₃ and the like per the definition of alkyl above.

The term “acyloxy” means —OC(O)C₁C₈alkyl and the like per the definition of alkyl above.

“Acylamino” means-N(H)C(O)C₁C₈alkyl and the like per the definition of alkyl above.

“Aryloxy” means —O(aryl), —O(substituted aryl), —O(heteroaryl) or —O(substituted heteroaryl).

“Arylamino” means —NH(aryl), —NH(substituted aryl), —NH(heteroaryl) or —NH(substituted heteroaryl), and the like.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

“Haloalkyl” refers to an alkyl group that is substituted with one or more halogen substituents, suitably from 1 to 6 substituents. Haloalkyl includes trifluoromethyl.

When “cycloalkyl” is used it refers to a non-aromatic, saturated, cyclic hydrocarbon ring containing the specified number of carbon atoms. So, for example, the term “C₃-C₈cycloalkyl” refers to a non-aromatic cyclic hydrocarbon ring having from three to eight carbon atoms. Exemplary “C₃-C₈cycloalkyl” groups useful in the present invention include, but are not limited to, cyclopropyl, cyclobutyl, cyclopentyl, cyclohexyl, cycloheptyl and cyclooctyl.

The term “C₅C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃C₈heterocycloalkyl” is used, it means a non-aromatic heterocyclic ring containing the specified number of ring atoms being, saturated or having one or more degrees of unsaturation and containing one or more heteroatom substitutions independently selected from O, S and N. Such a ring may be optionally fused to one or more other “heterocyclic” ring(s) or cycloalkyl ring(s). Examples are given herein below.

“Aryl” refers to optionally substituted monocyclic or polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl, and the like, as further illustrated below.

“Heteroaryl” means an optionally substituted aromatic monocyclic ring or polycarbocyclic fused ring system wherein at least one ring complies with Hückel's Rule, has the specified number of ring atoms, and that ring contains at least one heteratom independently selected from N, O and S. Examples of “heteroaryl” groups are given herein below.

The term “optionally” means that the subsequently described event(s) may or may not occur, and includes both event(s), which occur, and events that do not occur.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

### Formula II

In other embodiments, the EZH2 inhibitor is an inhibitor having Formula II, or a pharmaceutically acceptable salt thereof. Formula (II):

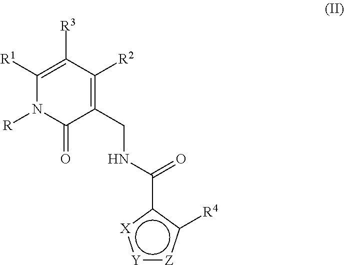

wherein:

X is O, N, S, CR⁶, or NR⁷;

Y is O, N, S, CR⁶, or NR⁷;

Z is CR⁵ or NR⁸; wherein when X is O, S, or NR⁷, Y is N or CR⁶ and Z is CR⁵; when Y is O, S, or NR⁷, X is N or CR⁶ and Z is CR⁵; and when Z is NR⁸, Y is N or CR⁶ and X is N or CR⁶;

R is hydrogen or (C₁-C₄)alkyl;

R¹, R², and R³ are each independently selected from the group consisting of hydrogen, (C₁-C₄)alkoxy, (C₁-C₈)alkyl, (C₁-C₄)alkoxy(C₁-C₄)alkyl-, halo(C₁-C₄)alkyl, (C₃-C₈)cycloalkyl, hydroxy(C₁-C₄)alkyl, (C₃-C₈)cycloalkyl(C₁-C₄)alkyl-, R^{a}O(O)CNH(C₁-C₄)alkyl-, (C₆-C₁₀)bicycloalkyl, heterocycloalkyl, heterocycloalkyl(C₁-C₄)alkyl-, aryl, aryl(C₁-C₄)alkyl, heteroaryl, heteroaryl(C₁-C₄)alkyl, halogen, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b}, wherein each (C₃-C₈)cycloalkyl, (C₆-C₁₀)bicycloalkyl, heterocycloalkyl, aryl, or heteroaryl is optionally substituted 1, 2, or 3 times, independently, by hydroxyl, halogen, nitro, (C₁-C₄)alkyl, cyano, (C₁-C₄)alkoxy, —NR^{a}R^{b} or —CO₂R^{a};

R⁴ is selected from the group consisting of hydrogen, (C₁-C₃)alkoxy, (C₁-C₃)alkyl, hydroxyl, halogen, cyano, (C₃-C₆)cycloalkyl, heterocycloalkyl, —NR^{a}R^{b}, halo(C₁-C₃)alkyl, and hydroxy(C₁-C₃)alkyl;

R⁵ is selected from the group consisting of (C₄-C₈)alkyl, (C₂-C₈)alkenyl, (C₃-C₈)alkoxy, (C₄-C₈)cycloalkyl, (C₃-C₈)cycloalkyl(C₁-C₂)alkyl-, (C₃-C₈)cycloalkyloxy-, heterocycloalkyl, heterocycloalkyl(C₁-C₂)alkyl-, heterocycloalkyloxy-, aryl, heteroaryl, and —NR^{a}R^{b}, wherein said (C₄-C₈)alkyl, (C₂-C₈)alkenyl, (C₃-C₈)alkoxy, (C₄-C₈)cycloalkyl, (C₃-C₈)cycloalkyl(C₁-C₂)alkyl-, (C₃-C₈)cycloalkyloxy-, heterocycloalkyl, heterocycloalkyl(C₁-C₂)alkyl-, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, heterocycloalkyl, aryl, or heteroaryl, wherein said (C₃-C₈)cycloalkyl, heterocycloalkyl, aryl, or heteroaryl is optionally substituted 1 or 2 times, independently, by halogen, (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-; or any 2 optional substituents on said (C₂-C₈)alkenyl taken together with the carbon atom(s) to which they are attached represent a 5-8 membered ring, optionally containing a heteroatom selected from oxygen, nitrogen, and sulfur, wherein said ring is optionally substituted 1 or 2 times, independently, by (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-;

R⁶ is selected from the group consisting of hydrogen, halogen, (C₁-C₈)alkyl, (C₁-C₄)alkoxy, —B(OH)₂, (C₃-C₈)cycloalkyl, (C₃-C₈)cycloalkyl(C₁-C₄)alkyl-, (C₆-C₁₀)bicycloalkyl, heterocycloalkyl, heterocycloalkyl(C₁-C₄)alkyl-, aryl, aryl(C₁-C₄)alkyl, heteroaryl, heteroaryl(C₁-C₄)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b}, wherein each cycloalkyl, bicycloalkyl, heterocycloalkyl, aryl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₈)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, or heteroaryl(C₁-C₄)alkyl;

R⁷ is selected from the group consisting of hydrogen, (C₁-C₈)alkyl, (C₃-C₈)cycloalkyl, (C₃-C₈)cycloalkyl(C₁-C₄)alkyl-, (C₆-C₁₀)bicycloalkyl, heterocycloalkyl, heterocycloalkyl(C₁-C₄)alkyl-, aryl, aryl(C₁-C₄)alkyl, heteroaryl, heteroaryl(C₁-C₄)alkyl, —C(O)R^{a}, —CO₂R^{a}, —C(O)NR^{a}R^{b}, —C(O)NR^{a}NR^{a}R^{b}, —SO₂R^{a}, —SO₂NR^{a}R^{b}, and R^{a}R^{b}N(C₁-C₄)alkyl-, wherein each cycloalkyl, bicycloalkyl, heterocycloalkyl, aryl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₈)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, or heteroaryl(C₁-C₄)alkyl;

R⁸ is selected from the group consisting of (C₄-C₈)alkyl, (C₄-C₈)cycloalkyl, heterocycloalkyl, heterocycloalkyl(C₁-C₂)alkyl-, aryl, and heteroaryl, wherein said (C₄-C₈)alkyl, (C₄-C₈)cycloalkyl, heterocycloalkyl, heterocycloalkyl(C₁-C₂)alkyl-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl;

each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and

R^{a} and R^{b} are each independently hydrogen, (C₁-C₄)alkyl, (C₁-C₄)alkoxy(C₁-C₄)alkyl-, (C₃-C₁₀)cycloalkyl, heterocycloalkyl, aryl, aryl(C₁-C₄)alkyl-, heteroaryl(C₁-C₄)alkyl-, or heteroaryl, wherein any said cycloalkyl, heterocycloalkyl, aryl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

or pharmaceutically acceptable salts thereof.

In one embodiment, this invention relates to compounds of Formula (II), wherein X is O, N, S, CR⁶, or NR⁷; Y is O, N, S, CR⁶, or NR⁷; wherein when X is O, S, or NR⁷, Y is N or CR⁶; and when Y is O, S, or NR⁷, X is N or CR⁶; and Z is CR⁵. In one embodiment, this invention relates to compounds of Formula (II), wherein X is O, S, or NR⁷; Y is N or CR⁶; and Z is CR⁵. In another embodiment, this invention relates to compounds of Formula (II), wherein X is O or S; Y is N or CR⁶; and Z is CR⁵. In another embodiment, this invention relates to compounds of Formula (II), wherein X is O or S; Y is CR⁶; and Z is CR⁵. In another embodiment, this invention relates to compounds of Formula (II), wherein X is O or S; Y is N; and Z is CR⁵. In another embodiment, this invention relates to compounds of Formula (II), wherein X is S; Y is CR⁶; and Z is CR⁵. In another embodiment, this invention relates to compounds of Formula (II), wherein Y is O, S, or NR⁷; X is N or CR⁶; and Z is CR⁵. In another embodiment, this invention relates to compounds of Formula (II), wherein Y is O or S; X is N or CR⁶; and Z is CR⁵. In another embodiment, this invention relates to compounds of Formula (II), wherein Y is O or S; X is CR⁶; and Z is CR⁵. In another embodiment, this invention relates to compounds of Formula (II), wherein Y is O or S; X is N; and Z is CR⁵. In another embodiment, this invention relates to compounds of Formula (II), wherein Y is S; X is CR⁶; and Z is CR⁵. In another embodiment, this invention relates to compounds of Formula (II), wherein Z is NR⁸; Y is N or CR⁶; and X is N or CR⁶.

In another embodiment, this invention relates to compounds of Formula (II), wherein R is hydrogen or methyl. In a specific embodiment, this invention relates to compounds of Formula (II), wherein R is methyl. In another specific embodiment, this invention relates to compounds of Formula (II), wherein R is hydrogen.

In another embodiment, this invention relates to compounds of Formula (II), wherein R¹, R², and R³ are each independently selected from the group consisting of hydrogen, (C₁-C₄)alkoxy, (C₁-C₄)alkyl, (C₁-C₄)alkoxy(C₁-C₄)alkyl-, halo(C₁-C₄)alkyl, (C₃-C₈)cycloalkyl, hydroxy(C₁-C₄)alkyl, (C₃-C₈)cycloalkyl(C₁-C₄)alkyl-, (C₁-C₄)alkylO(O)CNH(C₁-C₄)alkyl-, heterocycloalkyl, heterocycloalkyl(C₁-C₄)alkyl-, aryl, aryl(C₁-C₄)alkyl-, heteroaryl, and heteroaryl(C₁-C₄)alkyl-, wherein each (C₃-C₈)cycloalkyl, heterocycloalkyl, aryl, or heteroaryl is optionally substituted 1 or 2 times, independently, by hydroxyl, halogen, nitro, (C₁-C₄)alkyl, cyano, (C₁-C₄)alkoxy, —NH(C₁-C₄)alkyl, —N((C₁-C₄)alkyl)₂, or —CO₂(C₁-C₄)alkyl. In another embodiment, this invention relates to compounds of Formula (II), wherein R¹, R², and R³ are each independently selected from the group consisting of hydrogen, (C₁-C₄)alkoxy, (C₁-C₄)alkyl, (C₁-C₄)alkoxy(C₁-C₄)alkyl-, halo(C₁-C₄)alkyl, and hydroxy(C₁-C₄)alkyl.

In another embodiment, this invention relates to compounds of Formula (II), wherein R¹ and R² are each independently (C₁-C₄)alkoxy, (C₁-C₄)alkyl, or halo(C₁-C₄)alkyl.

In another embodiment, this invention relates to compounds of Formula (II), wherein R¹ and R² are each independently methyl, n-propyl, trifluoromethyl, or methoxy.

In another embodiment, this invention relates to compounds of Formula (II), wherein R¹ and R² are each independently (C₁-C₄)alkyl.

In a specific embodiment, this invention relates to compounds of Formula (II), wherein R¹ is methyl.

In another specific embodiment, this invention relates to compounds of Formula (II), wherein R² is methyl.

In another specific embodiment, this invention relates to compounds of Formula (II), wherein R¹ and R² are each methyl.

In another specific embodiment, this invention relates to compounds of Formula (II), wherein R³ is hydrogen.

In another embodiment, this invention relates to compounds of Formula (II), wherein R⁴ is selected from the group consisting of hydrogen, (C₁-C₃)alkyl, hydroxyl, halogen, halo(C₁-C₃)alkyl, and hydroxy(C₁-C₃)alkyl. In another embodiment, this invention relates to compounds of Formula (II), wherein R⁴ is (C₁-C₃)alkyl or halogen. In a specific embodiment, this invention relates to compounds of Formula (II), wherein R⁴ is methyl or chlorine. In another specific embodiment, this invention relates to compounds of Formula (II), wherein R⁴ is methyl.

In another embodiment, this invention relates to compounds of Formula (II), wherein R⁵ is selected from the group consisting of (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, heteroaryl, and —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl.

In another embodiment, this invention relates to compounds of Formula (II), wherein R⁵ is selected from the group consisting of (C₃-C₆)alkoxy, (C₃-C₆)cycloalkyloxy-, heterocycloalkyloxy-, heterocycloalkyl, —NH((C₃-C₆)cycloalkyl), —N((C₁-C₃)alkyl)((C₃-C₆)cycloalkyl), —NH(heterocycloalkyl), and —N((C₁-C₃)alkyl)(heterocycloalkyl), wherein any said (C₃-C₆)alkoxy, (C₃-C₆)cycloalkyloxy-, heterocycloalkyloxy-, heterocycloalkyl, or (C₃-C₆)cycloalkyl is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

In another embodiment, this invention relates to compounds of Formula (II), wherein R⁵ is selected from the group consisting of (C₃-C₆)alkoxy, (C₃-C₈)cycloalkyloxy-, and heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, or heteroaryl.

In another embodiment, this invention relates to compounds of Formula (II), wherein R⁵ is (C₃-C₆)cycloalkyloxy—which is optionally substituted 1, 2, or 3 times, independently, by halogen, —OR^{a}, —NR^{a}R^{b}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl. In another embodiment, this invention relates to compounds of Formula (II), wherein R⁵ is (C₃-C₆)cycloalkyloxy—which is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

In another embodiment, this invention relates to compounds of Formula (II), wherein R⁵ is heterocycloalkyloxy—which is optionally substituted 1, 2, or 3 times, independently, by halogen, —OR^{a}, —NR^{a}R^{b}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl. In another embodiment, this invention relates to compounds of Formula (II), wherein R⁵ is heterocycloalkyloxy—which is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

In another embodiment, this invention relates to compounds of Formula (II), wherein R⁵ is selected from the group consisting of cyclopentyloxy, cyclohexyloxy, pyrrolidinyloxy, piperidinyloxy, and tetrahydropyranyloxy, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, or pyrimidinyl, wherein R^{a} is (C₁-C₄)alkyl or phenyl(C₁-C₂)alkyl and R^{b} is hydrogen or (C₁-C₄)alkyl.

In another embodiment, this invention relates to compounds of Formula (II), wherein R⁵ is —NR^{a}R^{b}. In another embodiment, this invention relates to compounds of Formula (II), wherein R⁵ is —NR^{a}R^{b}; R^{a} is azetidinyl, oxetanyl, pyrrolidinyl, piperidinyl, piperazinyl, morpholinyl, thiomorpholinyl, or tetrahydropyranyl, each of which is optionally substituted 1 or 2 times, independently, by (C₁-C₄)alkyl; and R^{b} is hydrogen or (C₁-C₄)alkyl. In another embodiment, this invention relates to compounds of Formula (II), wherein R⁵ is —NR^{a}R^{b}; R^{a} is azetidinyl, oxetanyl, pyrrolidinyl, piperidinyl, piperazinyl, morpholinyl, thiomorpholinyl, or tetrahydropyranyl; and R^{b} is methyl or ethyl.

In another embodiment, this invention relates to compounds of Formula (II), wherein R⁵ is —NR^{a}R^{b}; R^{a} is cyclopentyl or cyclohexyl, each of which is optionally substituted by amino, —NH(C₁-C₄)alkyl, or —N((C₁-C₄)alkyl)₂; and R^{b} is hydrogen or (C₁-C₄)alkyl. In another embodiment, this invention relates to compounds of Formula (II), wherein R⁵ is —NR^{a}R^{b}; R^{a} is cyclopentyl or cyclohexyl, each of which is optionally substituted by —N((C₁-C₂)alkyl)₂; and R^{b} is methyl or ethyl. In another embodiment, this invention relates to compounds of Formula (II), wherein R⁵ is —NR^{a}R^{b}; R^{a} is cyclohexyl which is optionally substituted by amino, —NH(C₁-C₄)alkyl, or —N((C₁-C₄)alkyl)₂; and R^{b} is hydrogen or (C₁-C₄)alkyl. In another embodiment, this invention relates to compounds of Formula (II), wherein R⁵ is —NR^{a}R^{b}; R^{a} is cyclohexyl which is optionally substituted by —N((C₁-C₂)alkyl)₂; and R^{b} is methyl or ethyl.

In another embodiment, this invention relates to compounds of Formula (II), wherein R⁵ is (C₂-C₈)alkenyl which is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, heterocycloalkyl, aryl, or heteroaryl, wherein said (C₃-C₈)cycloalkyl, heterocycloalkyl, aryl, or heteroaryl is optionally substituted 1 or 2 times, independently, by halogen, (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-; or any 2 optional substituents on said (C₂-C₈)alkenyl taken together with the carbon atom(s) to which they are attached represent a 5-8 membered ring, optionally containing a heteroatom selected from oxygen, nitrogen, and sulfur, wherein said ring is optionally substituted 1 or 2 times, independently, by (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-.

In another embodiment, this invention relates to compounds of Formula (II), wherein R⁵ is (C₂-C₄)alkenyl which is optionally substituted 1 or 2 times, independently, by (C₃-C₆)cycloalkyl, 5- or 6-membered heterocycloalkyl, phenyl, or 5- or 6-membered heteroaryl, each of which is optionally substituted 1 or 2 times, independently, by halogen, (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-; or any 2 optional substituents on said (C₂-C₄)alkenyl taken together with the carbon atom(s) to which they are attached represent a 5-6 membered ring, optionally containing a heteroatom selected from oxygen, nitrogen, and sulfur, wherein said ring is optionally substituted by (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-.

In another embodiment, this invention relates to compounds of Formula (II), wherein R⁵ is (C₂-C₄)alkenyl which is optionally substituted by (C₃-C₆)cycloalkyl, 5- or 6-membered heterocycloalkyl, phenyl, or 5- or 6-membered heteroaryl, each of which is optionally substituted by halogen, (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-. In another embodiment, this invention relates to compounds of Formula (II), wherein R⁵ is (C₂-C₄)alkenyl which is optionally substituted by cyclopentyl, cyclohexyl, pyrrolidinyl, tetrahydrofuranyl, dihydrofuranyl, piperidinyl, tetrahydropyranyl, or dihydropyranyl, each of which is optionally substituted by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-. In another embodiment, this invention relates to compounds of Formula (II), wherein R⁵ is (C₂-C₄)alkenyl which is optionally substituted by cyclohexyl, piperidinyl, or tetrahydropyranyl, each of which is optionally substituted by (C₁-C₄)alkyl, halo(C₁-C₄)alkyl, amino, —NH(C₁-C₄)alkyl, or —N((C₁-C₄)alkyl)₂. In another embodiment, this invention relates to compounds of Formula (II), wherein R⁵ is (C₂-C₄)alkenyl which is optionally substituted by cyclopentyl or cyclohexyl, each of which is optionally substituted by amino, —NH(C₁-C₄)alkyl, or —N((C₁-C₄)alkyl)₂. In another embodiment, this invention relates to compounds of Formula (II), wherein R⁵ is (C₂-C₄)alkenyl which is optionally substituted by piperidinyl or tetrahydropyranyl.

In another embodiment, this invention relates to compounds of Formula (II), wherein R⁵ is (C₂-C₄)alkenyl containing 2 substituents which taken together with the carbon atom(s) to which they are attached represent a 5-6 membered ring, optionally containing a heteroatom selected from oxygen, nitrogen, and sulfur, wherein said ring is optionally substituted by (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-. In another embodiment, this invention relates to compounds of Formula (II), wherein R⁵ is (C₂-C₄)alkenyl containing 2 substituents which taken together with the carbon atom(s) to which they are attached represent a 5-6 membered ring, optionally containing a heteroatom selected from oxygen, nitrogen, and sulfur, wherein said ring is optionally substituted by (C₁-C₄)alkyl, halo(C₁-C₄)alkyl, amino, —NH(C₁-C₄)alkyl, or —N((C₁-C₄)alkyl)₂. In another embodiment, this invention relates to compounds of Formula (II), wherein R⁵ is (C₂-C₄)alkenyl containing 2 substituents which taken together with the carbon atom(s) to which they are attached represent a piperidinyl ring which is optionally substituted by (C₁-C₄)alkyl.

In another embodiment, this invention relates to compounds of Formula (II), wherein R⁵ is (C₃-C₈)cycloalkyl(C₁-C₂)alkyl- or heterocycloalkyl(C₁-C₂)alkyl-, each of which is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, heterocycloalkyl, aryl, or heteroaryl, wherein said (C₃-C₈)cycloalkyl, heterocycloalkyl, aryl, or heteroaryl is optionally substituted 1 or 2 times, independently, by halogen, (C₁-C₄)alkyl, halo(C₁-C₄)alkyl, —CO(C₁-C₄)alkyl, —CO₂(C₁-C₄)alkyl, —NR^{a}R^{b}, —NHCO₂R^{a}, hydroxyl, oxo, (C₁-C₄)alkoxy, or (C₁-C₄)alkoxy(C₁-C₄)alkyl-. In another embodiment, this invention relates to compounds of Formula (II), wherein R⁵ is (C₃-C₈)cycloalkyl(C₁-C₂)alkyl- or heterocycloalkyl(C₁-C₂)alkyl-, each of which is optionally substituted 1 or 2 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, heterocycloalkyl, aryl, or heteroaryl. In another embodiment, this invention relates to compounds of Formula (II), wherein R⁵ is (C₃-C₈)cycloalkyl(C₁-C₂)alkyl- or heterocycloalkyl(C₁-C₂)alkyl-, each of which is optionally substituted 1 or 2 times, independently, by —NR^{a}R^{b}, —NHCO₂R^{a}, (C₁-C₃)alkyl, or R^{a}R^{b}N(C₁-C₃)alkyl-. In another embodiment, this invention relates to compounds of Formula (II), wherein R⁵ is (C₃-C₆)cycloalkyl(C₁-C₂)alkyl- or heterocycloalkyl(C₁-C₂)alkyl-, each of which is optionally substituted 1 or 2 times, independently, by (C₁-C₃)alkyl, amino, —NH(C₁-C₄)alkyl, or —N((C₁-C₄)alkyl)₂, wherein said heterocycloalkyl moiety is monocyclic.

In another embodiment, this invention relates to compounds of Formula (II), wherein R⁵ is (C₅-C₆)cycloalkyl(C₁-C₂)alkyl- or heterocycloalkyl(C₁-C₂)alkyl-, each of which is optionally substituted 1 or 2 times, independently, by (C₁-C₃)alkyl, amino, —NH(C₁-C₄)alkyl, or —N((C₁-C₄)alkyl)₂, wherein said heterocycloalkyl moiety is selected from the group consisting of piperidinyl, piperazinyl, morpholinyl, and tetrahydropyranyl. In another embodiment, this invention relates to compounds of Formula (II), wherein R⁵ is cyclohexylmethyl which is optionally substituted 1 or 2 times, independently, by (C₁-C₃)alkyl, amino, —NH(C₁-C₄)alkyl, or —N((C₁-C₄)alkyl)₂. In another embodiment, this invention relates to compounds of Formula (II), wherein R⁵ is piperidin-1-ylmethyl which is optionally substituted 1 or 2 times, independently, by (C₁-C₃)alkyl, amino, —NH(C₁-C₄)alkyl, or —N((C₁-C₄)alkyl)₂.

In another embodiment, this invention relates to compounds of Formula (II), wherein R⁶ is selected from the group consisting of hydrogen, —SO₂(C₁-C₄)alkyl, halogen, (C₁-C₆)alkyl, (C₁-C₄)alkoxy, phenyl, heteroaryl, and cyano, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (II), wherein R⁶ is selected from the group consisting of hydrogen, cyano, halogen, (C₁-C₄)alkoxy, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, or triazinyl is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (II), wherein R⁶ is phenyl which is optionally substituted by —NR^{a}R^{b} or R^{a}R^{b}N(C₁-C₄)alkyl-.

In another embodiment, this invention relates to compounds of Formula (II), wherein R⁶ is pyridinyl which is optionally substituted by —NR^{a}R^{b} or R^{a}R^{b}N(C₁-C₄)alkyl-.

In another embodiment, this invention relates to compounds of Formula (II), wherein R⁶ is hydrogen, halogen, (C₁-C₄)alkyl, or (C₁-C₄)alkoxy. In another embodiment, this invention relates to compounds of Formula (II), wherein R⁶ is hydrogen or halogen. In a specific embodiment, this invention relates to compounds of Formula (II), wherein R⁶ is hydrogen, fluorine, chlorine, or bromine. In a specific embodiment, this invention relates to compounds of Formula (II), wherein R⁶ is hydrogen or chlorine. In a more specific embodiment, this invention relates to compounds of Formula (II), wherein R⁶ is chlorine. In another specific embodiment, this invention relates to compounds of Formula (II), wherein R⁶ is hydrogen.

In another embodiment, this invention relates to compounds of Formula (II), wherein R⁷ is selected from the group consisting of hydrogen, (C₁-C₆)alkyl, (C₃-C₆)cycloalkyl, phenyl, and heteroaryl, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (II), wherein R⁷ is hydrogen or (C₁-C₄)alkyl.

In another embodiment, this invention relates to compounds of Formula (II), wherein R⁷ is selected from the group consisting of furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, or triazinyl is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (II), wherein R⁷ is phenyl which is optionally substituted by —NR^{a}R^{b} or R^{a}R^{b}N(C₁-C₄)alkyl-.

In another embodiment, this invention relates to compounds of Formula (II), wherein R⁷ is pyridinyl which is optionally substituted by —NR^{a}R^{b} or R^{a}R^{b}N(C₁-C₄)alkyl-.

In another embodiment, this invention relates to compounds of Formula (II), wherein R⁸ is selected from the group consisting of (C₄-C₈)alkyl, (C₄-C₈)cycloalkyl, heterocycloalkyl, aryl, and heteroaryl, wherein said (C₄-C₈)alkyl, (C₄-C₈)cycloalkyl, heterocycloalkyl, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl.

In another embodiment, this invention relates to compounds of Formula (II), wherein R⁸ is selected from the group consisting of (C₄-C₆)alkyl, (C₄-C₆)cycloalkyl, heterocycloalkyl, and phenyl, wherein said (C₄-C₆)alkyl, (C₄-C₆)cycloalkyl, heterocycloalkyl, or phenyl is optionally substituted 1 or 2 times, independently, by —OR^{a}, —NR^{a}R^{b}, —NHCO₂R^{a}, (C₁-C₃)alkyl, R^{a}R^{b}N(C₁-C₃)alkyl-, R^{a}O(C₁-C₃)alkyl-, —CO₂R^{a}, —C(O)NR^{a}R^{b}, or —SO₂NR^{a}R^{b}.

In a particular embodiment, this invention relates to compounds of Formula (II), wherein:

R is hydrogen or methyl;

X is O, S, or NR⁷;

Y is N or CR⁶;

Z is CR⁵;

R¹ and R² are each independently (C₁-C₄)alkyl;

R³ is hydrogen;

R⁴ is methyl or chlorine;

R⁵ is selected from the group consisting of (C₃-C₆)alkoxy, (C₃-C₈)cycloalkyloxy-, and heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b} phenyl, or heteroaryl;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is O, S, or NR⁷;

Y is N or CR⁶;

Z is CR⁵;

R¹ and R² are each independently (C₁-C₄)alkyl;

R³ is hydrogen;

R⁴ is methyl or chlorine;

R⁵ is —NR^{a}R^{b};

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is O, S, or NR⁷;

Y is N or CR⁶;

Z is CR⁵;

R¹ and R² are each independently (C₁-C₄)alkyl;

R³ is hydrogen;

R⁴ is methyl or chlorine;

R⁵ is (C₂-C₄)alkenyl which is optionally substituted by cyclohexyl, piperidinyl, or tetrahydropyranyl, each of which is optionally substituted by (C₁-C₄)alkyl, halo(C₁-C₄)alkyl, amino, —NH(C₁-C₄)alkyl, or —N((C₁-C₄)alkyl)₂;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is O, S, or NR⁷;

Y is N or CR⁶;

Z is CR⁵;

R¹ and R² are each independently (C₁-C₄)alkyl;

R³ is hydrogen;

R⁴ is methyl or chlorine;

R⁵ is (C₅-C₆)cycloalkyl(C₁-C₂)alkyl- or heterocycloalkyl(C₁-C₂)alkyl-, each of which is optionally substituted 1 or 2 times, independently, by (C₁-C₃)alkyl, amino, —NH(C₁-C₄)alkyl, or —N((C₁-C₄)alkyl)₂, wherein said heterocycloalkyl moiety is selected from the group consisting of piperidinyl, piperazinyl, morpholinyl, and tetrahydropyranyl;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is N or CR⁶;

Y is O, S, or NR⁷;

Z is CR⁵;

R¹ and R² are each independently (C₁-C₄)alkyl;

R³ is hydrogen;

R⁴ is methyl or chlorine;

R⁵ is selected from the group consisting of (C₃-C₆)alkoxy, (C₃-C₈)cycloalkyloxy-, and heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, or heteroaryl;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is N or CR⁶;

Y is O, S, or NR⁷;

Z is CR⁵;

R¹ and R² are each independently (C₁-C₄)alkyl;

R³ is hydrogen;

R⁴ is methyl or chlorine;

R⁵ is —NR^{a}R^{b};

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is N or CR6;

Y is O, S, or NR⁷;

Z is CR⁵;

R¹ and R² are each independently (C₁-C₄)alkyl;

R³ is hydrogen;

R⁴ is methyl or chlorine;

R⁵ is (C₂-C₄)alkenyl which is optionally substituted by cyclohexyl, piperidinyl, or tetrahydropyranyl, each of which is optionally substituted by (C₁-C₄)alkyl, halo(C₁-C₄)alkyl, amino, —NH(C₁-C₄)alkyl, or —N((C₁-C₄)alkyl)₂;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another particular embodiment, this invention relates to compounds of Formula (II), wherein:

R is hydrogen or methyl;

X is N or CR6;

Y is O, S, or NR⁷;

Z is CR⁵;

R¹ and R² are each independently (C₁-C₄)alkyl;

R³ is hydrogen;

R⁴ is methyl or chlorine;

R⁵ is (C₅-C₆)cycloalkyl(C₁-C₂)alkyl- or heterocycloalkyl(C₁-C₂)alkyl-, each of which is optionally substituted 1 or 2 times, independently, by (C₁-C₃)alkyl, amino, —NH(C₁-C₄)alkyl, or —N((C₁-C₄)alkyl)₂, wherein said heterocycloalkyl moiety is selected from the group consisting of piperidinyl, piperazinyl, morpholinyl, and tetrahydropyranyl;

R⁶ is hydrogen, halogen, (C₁-C₄)alkyl, or (C₁-C₄)alkoxy; and

R⁷ is hydrogen or (C₁-C₄)alkyl;

or pharmaceutically acceptable salts thereof.

In another embodiment, this invention also relates to compounds of Formula (III):

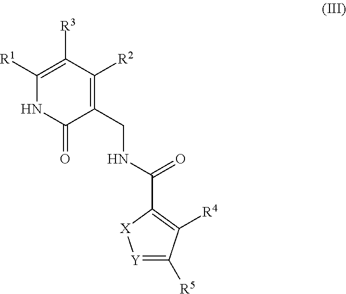

or pharmaceutically acceptable salts thereof, wherein X is O, S, or NR⁷; Y is N or CR⁶; and R, R², R³, R⁴, R⁵, R⁶, and R⁷ are defined according to Formula (II). In another embodiment, this invention relates to compounds of Formula (III), wherein X is O or S and Y is N or CR⁶. In another embodiment, this invention relates to compounds of Formula (III), wherein X is O or S and Y is CR⁶. In another embodiment, this invention relates to compounds of Formula (III), wherein X is O or S and Y is N. In another embodiment, this invention relates to compounds of Formula (III), wherein X is S and Y is CR⁶.

In another embodiment, this invention also relates to compounds of Formula (III)(a):

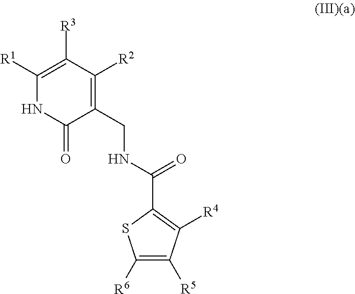

or pharmaceutically acceptable salts thereof, wherein R¹, R², R³, R⁴, R, and R⁶ are defined according to Formula (II).

In another embodiment, this invention also relates to compounds of Formula (IV):

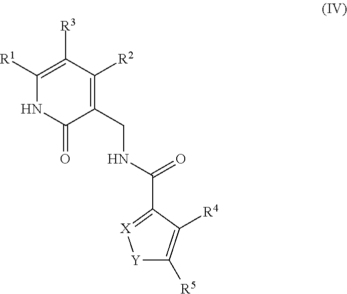

or pharmaceutically acceptable salts thereof, wherein Y is O, S, or NR⁷; X is N or CR⁶; and R¹, R², R³, R⁴, R⁵, R⁶, and R⁷ are defined according to Formula (II). In another embodiment, this invention relates to compounds of Formula (IV), wherein Y is O or S and X is N or CR⁶. In another embodiment, this invention relates to compounds of Formula (IV), wherein Y is O or S and X is CR⁶. In another embodiment, this invention relates to compounds of Formula (IV), wherein Y is O or S and X is N. In another embodiment, this invention relates to compounds of Formula (IV), wherein Y is S and X is CR⁶.

In another embodiment, this invention also relates to compounds of Formula (IV)(a):

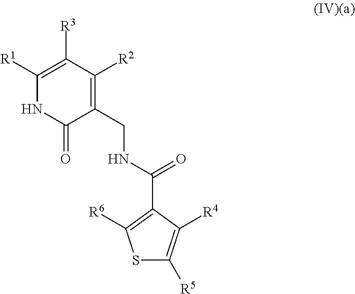

or pharmaceutically acceptable salts thereof, wherein R¹, R², R³, R⁴, R⁵, and R⁶ are defined according to Formula (II).

In another embodiment, this invention also relates to compounds of Formula (IV):

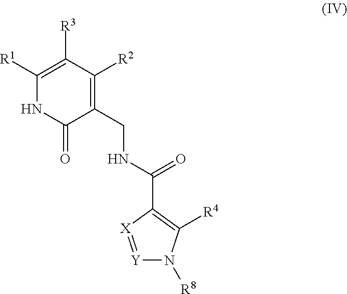

or pharmaceutically acceptable salts thereof, wherein X is N or CR⁶; Y is N or CR⁶; and R¹, R², R³, R⁴, R⁶, and R⁸ are defined according to Formula (II). In another embodiment, this invention relates to compounds of Formula (IV), wherein X is N and Y is CR⁶. In another embodiment, this invention relates to compounds of Formula (IV), wherein X is CR⁶ and Y is N. In another embodiment, this invention relates to compounds of Formula (IV), wherein X and Y are each independently CR⁶. In another embodiment, this invention relates to compounds of Formula (IV), wherein X and Y are each N.

Specific EZH2 inhibitors for use in the methods or as a pharmaceutical composition for use in the treatment of cancer as disclosed herein include:
N-((4,6-dimethyl-2-oxo-1,2-dihydropyridin-3-yl)methyl)-4-isopropoxy-3-methylthiophene-2-carboxamide;
N-((4,6-dimethyl-2-oxo-1,2-dihydropyridin-3-yl)methyl)-4-(ethyl(tetrahydro-2H-pyran-4-yl)amino)-3-methylthiophene-2-carboxamide;
5-chloro-N-((4,6-dimethyl-2-oxo-1,2-dihydropyridin-3-yl)methyl)-4-(ethyl(tetrahydro-2H-pyran-4-yl)amino)-3-methylthiophene-2-carboxamide;
N-((4,6-dimethyl-2-oxo-1,2-dihydropyridin-3-yl)methyl)-4-(((trans)-4-(dimethylamino)cyclohexyl)(ethyl)amino)-3-methylthiophene-2-carboxamide;
N-((4,6-dimethyl-2-oxo-1,2-dihydropyridin-3-yl)methyl)-4-(((cis)-4-(dimethylamino)cyclohexyl)(ethyl)amino)-3-methylthiophene-2-carboxamide;
N-((4,6-dimethyl-2-oxo-1,2-dihydropyridin-3-yl)methyl)-5-(ethyl(tetrahydro-2H-pyran-4-yl)amino)-4-methylthiophene-3-carboxamide;
N-((4,6-dimethyl-2-oxo-1,2-dihydropyridin-3-yl)methyl)-5-(((cis)-4-(dimethylamino)cyclohexyl)(ethyl)amino)-4-methylthiophene-3-carboxamide;
N-((4,6-dimethyl-2-oxo-1,2-dihydropyridin-3-yl)methyl)-5-(((trans)-4-(dimethylamino)cyclohexyl)(ethyl)amino)-4-methylthiophene-3-carboxamide;
tert-butyl 4-((4-(((4,6-dimethyl-2-oxo-1,2-dihydropyridin-3-yl)methyl)carbamoyl)-3-methylthiophen-2-yl)(ethyl)amino)piperidine-1-carboxylate;
N-((4,6-dimethyl-2-oxo-1,2-dihydropyridin-3-yl)methyl)-5-(ethyl(piperidin-4-yl)amino)-4-methylthiophene-3-carboxamide;
N-((4,6-dimethyl-2-oxo-1,2-dihydropyridin-3-yl)methyl)-5-(1-(4-(dimethylamino)piperidin-1-yl)ethyl)thiophene-3-carboxamide;
N-[(4,6-dimethyl-2-oxo-1,2-dihydro-3-pyridinyl)methyl]-5-[ethyl(tetrahydro-2H-pyran-4-yl)amino]-1,4-dimethyl-1H-pyrazole-3-carboxamide;
5-(ethyl(tetrahydro-2H-pyran-4-yl)amino)-4-methyl-N-((6-methyl-2-oxo-4-(trifluoromethyl)-1,2-dihydropyridin-3-yl)methyl)thiophene-3-carboxamide;
5-(((trans)-4-(dimethylamino)cyclohexyl)(ethyl)amino)-4-methyl-N-((6-methyl-2-oxo-4-propyl-1,2-dihydropyridin-3-yl)methyl)thiophene-3-carboxamide;
5-(((trans)-4-(dimethylamino)cyclohexyl)(ethyl)amino)-4-methyl-N-((4-methyl-2-oxo-6-(trifluoromethyl)-1,2-dihydropyridin-3-yl)methyl)thiophene-3-carboxamide;
5-(((trans)-4-(dimethylamino)cyclohexyl)(ethyl)amino)-N-((4-methoxy-6-methyl-2-oxo-1,2-dihydropyridin-3-yl)methyl)-4-methylthiophene-3-carboxamide;
2-bromo-N-((4,6-dimethyl-2-oxo-1,2-dihydropyridin-3-yl)methyl)-5-(ethyl(tetrahydro-2H-pyran-4-yl)amino)-4-methylthiophene-3-carboxamide;
tert-butyl 4-((4-(((4,6-dimethyl-2-oxo-1,2-dihydropyridin-3-yl)methyl)carbamoyl)-3-methylthiophen-2-yl)(hydroxy)methyl)piperidine-1-carboxylate;
N-((4,6-dimethyl-2-oxo-1,2-dihydropyridin-3-yl)methyl)-5-(hydroxy(piperidin-4-yl)methyl)-4-methylthiophene-3-carboxamide;
N-((4,6-dimethyl-2-oxo-1,2-dihydropyridin-3-yl)methyl)-4-methyl-5-(1-(tetrahydro-2H-pyran-4-yl)pyrrolidin-2-yl)thiophene-3-carboxamide;
N-((4,6-dimethyl-2-oxo-1,2-dihydropyridin-3-yl)methyl)-4-methyl-5-(1-(piperidin-4-yl)-1H-pyrazol-4-yl)thiophene-3-carboxamide;
tert-butyl 3-(4-(((4,6-dimethyl-2-oxo-1,2-dihydropyridin-3-yl)methyl)carbamoyl)-3-methylthiophen-2-yl)pyrrolidine-1-carboxylate;
N-((4,6-dimethyl-2-oxo-1,2-dihydropyridin-3-yl)methyl)-4-methyl-5-(2-methylpyrrolidin-1-yl)thiophene-3-carboxamide;
N-((4,6-dimethyl-2-oxo-1,2-dihydropyridin-3-yl)methyl)-4-methyl-5-(1-morpholinoethyl)thiophene-3-carboxamide;
N-((4,6-dimethyl-2-oxo-1,2-dihydropyridin-3-yl)methyl)-5-(1-(4-(dimethylamino)piperidin-1-yl)ethyl)-4-methylthiophene-3-carboxamide;
N-((4,6-dimethyl-2-oxo-1,2-dihydropyridin-3-yl)methyl)-5-(1-(4-(dimethylamino)piperidin-1-yl)propyl)-4-methylthiophene-3-carboxamide;
N-((4,6-dimethyl-2-oxo-1,2-dihydropyridin-3-yl)methyl)-4-methyl-5-(1-morpholinopropyl)thiophene-3-carboxamide;
N-((4,6-dimethyl-2-oxo-1,2-dihydropyridin-3-yl)methyl)-5-(ethyl(tetrahydro-2H-pyran-4-yl)amino)thiophene-2-carboxamide;
5-(ethyl(tetrahydro-2H-pyran-4-yl)amino)-4-methyl-N-((1,4,6-trimethyl-2-oxo-1,2-dihydropyridin-3-yl)methyl)thiophene-3-carboxamide;
5-(((trans)-4-(dimethylamino)cyclohexyl)(ethyl)amino)-4-methyl-N-((1,4,6-trimethyl-2-oxo-1,2-dihydropyridin-3-yl)methyl)thiophene-3-carboxamide;
(E)-N-((4,6-dimethyl-2-oxo-1,2-dihydropyridin-3-yl)methyl)-4-methyl-5-(1-(piperidin-4-yl)prop-1-en-1-yl)thiophene-3-carboxamide;
tert-butyl ((trans)-4-((E)-1-(4-(((4,6-dimethyl-2-oxo-1,2-dihydropyridin-3-yl)methyl)carbamoyl)-3-methylthiophen-2-yl)prop-1-en-1-yl)cyclohexyl)carbamate;
5-((E)-1-((trans)-4-aminocyclohexyl)prop-1-en-1-yl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-((E)-1-((trans)-4-(dimethylamino)cyclohexyl)prop-1-en-1-yl)-4-methylthiophene-3-carboxamide;
5-(((trans)-4-aminocyclohexyl)(hydroxy)methyl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-5-(((trans)-4-(dimethylamino)cyclohexyl)(hydroxy)methyl)-4-methylthiophene-3-carboxamide;
tert-butyl 4-(1-(4-(((4,6-dimethyl-2-oxo-1,2-dihydropyridin-3-yl)methyl)carbamoyl)-3-methylthiophen-2-yl)propyl)piperidine-1-carboxylate;
N-((4,6-dimethyl-2-oxo-1,2-dihydropyridin-3-yl)methyl)-4-methyl-5-(1-(piperidin-4-yl)propyl)thiophene-3-carboxamide;
(S)-(−)-N-((4,6-dimethyl-2-oxo-1,2-dihydropyridin-3-yl)methyl)-5-(1-(4-(dimethylamino)piperidin-1-yl)propyl)-4-methylthiophene-3-carboxamide;
(R)-(+)-N-((4,6-dimethyl-2-oxo-1,2-dihydropyridin-3-yl)methyl)-5-(1-(4-(dimethylamino)piperidin-1-yl)propyl)-4-methylthiophene-3-carboxamide;
5-(1-((trans)-4-aminocyclohexyl)propyl)-N-((4,6-dimethyl-2-oxo-1,2-dihydropyridin-3-yl)methyl)-4-methylthiophene-3-carboxamide;
(−)-N-((4,6-dimethyl-2-oxo-1,2-dihydropyridin-3-yl)methyl)-5-(1-((trans)-4-(dimethylamino)cyclohexyl)propyl)-4-methylthiophene-3-carboxamide;
(+)-N-((4,6-dimethyl-2-oxo-1,2-dihydropyridin-3-yl)methyl)-5-(1-((trans)-4-(dimethylamino)cyclohexyl)propyl)-4-methylthiophene-3-carboxamide;
(+)-N-((4,6-dimethyl-2-oxo-1,2-dihydropyridin-3-yl)methyl)-5-(1-((cis)-4-(dimethylamino)cyclohexyl)propyl)-4-methylthiophene-3-carboxamide;
(−)-N-((4,6-dimethyl-2-oxo-1,2-dihydropyridin-3-yl)methyl)-5-(1-((cis)-4-(dimethylamino)cyclohexyl)propyl)-4-methylthiophene-3-carboxamide;
N-((4,6-dimethyl-2-oxo-1,2-dihydropyridin-3-yl)methyl)-4-methyl-5-(1-(piperidin-4-ylidene)propyl)thiophene-3-carboxamide;
N-((4,6-dimethyl-2-oxo-1,2-dihydropyridin-3-yl)methyl)-4-methyl-5-(1-(piperidin-4-yl)vinyl)thiophene-3-carboxamide;
2-bromo-N-((4,6-dimethyl-2-oxo-1,2-dihydropyridin-3-yl)methyl)-5-(((trans)-4-(dimethylamino)cyclohexyl)(ethyl)amino)-4-methylthiophene-3-carboxamide;
2-cyano-N-((4,6-dimethyl-2-oxo-1,2-dihydropyridin-3-yl)methyl)-5-(((trans)-4-(dimethylamino)cyclohexyl)(ethyl)amino)-4-methylthiophene-3-carboxamide;
2-bromo-5-(diethylamino)-N-((4,6-dimethyl-2-oxo-1,2-dihydropyridin-3-yl)methyl)-4-methylthiophene-3-carboxamide;
2-chloro-N-((4,6-dimethyl-2-oxo-1,2-dihydropyridin-3-yl)methyl)-5-(ethyl(tetrahydro-2H-pyran-4-yl)amino)-4-methylthiophene-3-carboxamide;
N-((4,6-dimethyl-2-oxo-1,2-dihydropyridin-3-yl)methyl)-5-(ethyl(tetrahydro-2H-pyran-4-yl)amino)-2,4-dimethylthiophene-3-carboxamide;
2-cyano-N-((4,6-dimethyl-2-oxo-1,2-dihydropyridin-3-yl)methyl)-5-(ethyl(tetrahydro-2H-pyran-4-yl)amino)-4-methylthiophene-3-carboxamide;
N-((4,6-dimethyl-2-oxo-1,2-dihydropyridin-3-yl)methyl)-5-(ethyl(tetrahydro-2H-pyran-4-yl)amino)-4-methyl-2-(4-methylpiperazin-1-yl)thiophene-3-carboxamide;
tert-butyl 3-(3-(((4,6-dimethyl-2-oxo-1,2-dihydropyridin-3-yl)methyl)carbamoyl)-5-(ethyl(tetrahydro-2H-pyran-4-yl)amino)-4-methylthiophen-2-yl)pyrrolidine-1-carboxylate;
N-((4,6-dimethyl-2-oxo-1,2-dihydropyridin-3-yl)methyl)-5-(ethyl(tetrahydro-2H-pyran-4-yl)amino)-4-methyl-2-(pyrrolidin-3-yl)thiophene-3-carboxamide;
tert-butyl 4-(3-(((4,6-dimethyl-2-oxo-1,2-dihydropyridin-3-yl)methyl)carbamoyl)-5-(ethyl(tetrahydro-2H-pyran-4-yl)amino)-4-methylthiophen-2-yl)piperidine-1-carboxylate;
N-((4,6-dimethyl-2-oxo-1,2-dihydropyridin-3-yl)methyl)-5-(ethyl(tetrahydro-2H-pyran-4-yl)amino)-4-methyl-2-(piperidin-4-yl)thiophene-3-carboxamide;
N-((4,6-dimethyl-2-oxo-1,2-dihydropyridin-3-yl)methyl)-5-(ethyl(tetrahydro-2H-pyran-4-yl)amino)-4-methyl-2-(1-methyl-1,2,3,6-tetrahydropyridin-4-yl)thiophene-3-carboxamide;
N-((4,6-dimethyl-2-oxo-1,2-dihydropyridin-3-yl)methyl)-5-(ethyl(tetrahydro-2H-pyran-4-yl)amino)-4-methyl-2-(1-(methylsulfonyl)pyrrolidin-3-yl)thiophene-3-carboxamide;
N-((4,6-dimethyl-2-oxo-1,2-dihydropyridin-3-yl)methyl)-5-(ethyl(tetrahydro-2H-pyran-4-yl)amino)-4-methyl-2-(1-(methylsulfonyl)piperidin-4-yl)thiophene-3-carboxamide;
N-((4,6-dimethyl-2-oxo-1,2-dihydropyridin-3-yl)methyl)-5-(ethyl((trans)-4-(ethyl(methyl)amino)cyclohexyl)amino)-4-methylthiophene-3-carboxamide;
ethyl (4-(((4,6-dimethyl-2-oxo-1,2-dihydropyridin-3-yl)methyl)carbamoyl)-3-methylthiophen-2-yl)(ethyl)carbamate;
N-((4,6-dimethyl-2-oxo-1,2-dihydropyridin-3-yl)methyl)-5-(ethyl(1-isopropylpiperidin-4-yl)amino)-4-methylthiophene-3-carboxamide;
N-((4,6-dimethyl-2-oxo-1,2-dihydropyridin-3-yl)methyl)-5-(ethyl(3-fluoropiperidin-4-yl)amino)-4-methylthiophene-3-carboxamide;
N-((4,6-dimethyl-2-oxo-1,2-dihydropyridin-3-yl)methyl)-4-methyl-5-(2-(piperidin-4-yl)pyrrolidin-1-yl)thiophene-3-carboxamide;
5-([2,4′-bipiperidin]-1-yl)-N-((4,6-dimethyl-2-oxo-1,2-dihydropyridin-3-yl)methyl)-4-methylthiophene-3-carboxamide;
N-((4,6-dimethyl-2-oxo-1,2-dihydropyridin-3-yl)methyl)-4-methyl-5-(1,2,3,4-tetrahydroisoquinolin-5-yl)thiophene-3-carboxamide;
(Z)—N-((4,6-dimethyl-2-oxo-1,2-dihydropyridin-3-yl)methyl)-4-methyl-5-(5-morpholinopent-2-en-3-yl)thiophene-3-carboxamide;
(Z)—N-((4,6-dimethyl-2-oxo-1,2-dihydropyridin-3-yl)methyl)-4-methyl-5-(6-(4-methylpiperazin-1-yl)hex-2-en-3-yl)thiophene-3-carboxamide;
N-((4,6-dimethyl-2-oxo-1,2-dihydropyridin-3-yl)methyl)-4-methyl-5-(6-(4-methylpiperazin-1-yl)hexan-3-yl)thiophene-3-carboxamide;
(Z)—N-((4,6-dimethyl-2-oxo-1,2-dihydropyridin-3-yl)methyl)-5-(6-(4-(dimethylamino)piperidin-1-yl)hex-2-en-3-yl)-4-methylthiophene-3-carboxamide;
N-((4,6-dimethyl-2-oxo-1,2-dihydropyridin-3-yl)methyl)-5-(ethyl(tetrahydro-2H-pyran-4-yl)amino)-2-(furan-3-yl)-4-methylthiophene-3-carboxamide;
N-((4,6-dimethyl-2-oxo-1,2-dihydropyridin-3-yl)methyl)-5-(ethyl(tetrahydro-2H-pyran-4-yl)amino)-2-(furan-2-yl)-4-methylthiophene-3-carboxamide;
N-((4,6-dimethyl-2-oxo-1,2-dihydropyridin-3-yl)methyl)-5-(((trans)-4-(dimethylamino)cyclohexyl)(ethyl)amino)-2-(furan-3-yl)-4-methylthiophene-3-carboxamide; and
N-((4,6-dimethyl-2-oxo-1,2-dihydropyridin-3-yl)methyl)-5-(ethyl(tetrahydro-2H-pyran-4-yl)amino)-4-methyl-2-(1-methyl-1H-pyrazol-4-yl)thiophene-3-carboxamide;

or pharmaceutically acceptable salts thereof.

### Pharmaceutical Formulations

While it is possible that, the compound of the present invention, as well as pharmaceutically acceptable salts and solvates thereof, may be administered as the raw chemical, it is also possible to present the active ingredient as a pharmaceutical composition. Accordingly, embodiments of the invention further provide pharmaceutical compositions, which include therapeutically effective amounts of a compound of Formula (I), or Compound A, or Compound B and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of Formula I, Compound A, or Compound B with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 800 mg, of a compound of the formula (I) depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known by one of skill in the art, e.g. in the pharmacy art

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

Dosage unit forms can also be in the form for i.v. delivery, of which one of skill in the art is capable of providing.

Dosage unit forms, e.g. for i.v. delivery, can also be in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines or other forms familiar to one of skill in the art.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of formula (I) or a pharmaceutically acceptable salt thereof will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) or a salt thereof for the treatment of a cancerous condition such as those described herein will generally be in the range of 0.1 to 100 mg/kg body weight of recipient (mammal) per day and more usually in the range of 1 to 12 mg/kg body weight per day. Thus, for a 70 kg adult mammal, the actual amount per day would usually be from 70 to 840 mg and this amount may be given in a single dose per day or more usually in a number (such as two, three, four, five or six) of sub-doses per day such that the total daily dose is the same. An effective amount of a salt or solvate thereof may be determined as a proportion of the effective amount of the compound of formula (I) per se. It is envisaged that similar dosages would be appropriate for treatment of the other conditions referred to above.

The amount of administered or prescribed compound according to these aspects of the present invention will depend upon a number of factors including, for example, the age and weight of the patient, the precise condition requiring treatment, the severity of the condition, the nature of the formulation, and the route of administration. Ultimately, the amount will be at the discretion of the attendant physician.

### Combinations and Additional Anti-Neoplastic Agents

In certain embodiments, the methods of the present invention further comprise administering one or more additional anti-neoplastic agents.

When an EZH2 inhibitor such as, but not limited to, Formula I, Compound A, or Compound B, or Formula II, III, or IV, or any of the specific EZH2 inhibitors listed herein is administered for the treatment of cancer, the term “co-administering” and derivatives thereof as used herein is meant either simultaneous administration or any manner of separate sequential administration of an EZH2 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. If the administration is not simultaneous, the compounds are administered in a close time proximity to each other. Furthermore, it does not matter if the compounds are administered in the same dosage form, e.g. one compound may be administered topically or intraveneously (i.v.) and another compound may be administered orally.

Typically, any anti-neoplastic agent that has activity versus a susceptible tumor or cancer (e.g. lymphoma) being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, any treatment for lymphoma, such as: R-CHOP, the five component treatment for non-Hodgkin's lymphoma, comprising: Rituximab, Cyclophosphamide, a DNA alkylating cross-linking agent; Hydroxydaunorubicin (i.e. doxorubicin or Adriamycin), a DNA intercalating agent; Oncovin (vincristine), which inhibits cell division by binding to the protein tubulin, and the corticosteroids Prednisone or prednisolone; CHOP, R-CVP (similar to R-CHOP, comprises rituximab, cyclophosphamide, vincristine, and prednisolone/prednisone), CVP; bortezomib; bendamustin; alemtuzumab; and radioimmunotherapy (e. ibritumomab (Zevalin), tositumomab (Bexxar)).

Other typical anti-neoplastic agents useful in the present invention include, but are not limited to. Class I and Class II histone deacetylase (HDAC) inhibitors (e.g., vorinostat), DNA methylase inhibitors (e.g. decitabine or azacitidine), histone acetyltransferase (HAT) inhibitors (e.g. p300 and PCAF inhibitors), anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present EZH2 inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3 S)—N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4‘-deoxy-C’-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula F following, currently under development, including the racemic mixture (R,S) form as well as the R and S enantiomers:

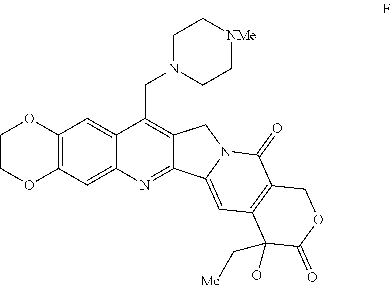 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Letrozole (trade name Femara) is an oral non-steroidal aromatase inhibitor for the treatment of hormonally-responsive breast cancer after surgery. Estrogens are produced by the conversion of androgens through the activity of the aromatase enzyme. Estrogens then bind to an estrogen receptor, which causes cells to divide. Letrozole prevents the aromatase from producing estrogens by competitive, reversible binding to the heme of its cytochrome P450 unit. The action is specific, and letrozole does not reduce production of mineralo- or corticosteroids.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and Bennett, C. F. and Cowsert, L. M. BioChim. Biophys. Acta, (1999) 1489(1):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Pazopanib which commercially available as VOTRIENT® is a tyrosine kinase inhibitor (TKI). Pazopanib is presented as the hydrochloride salt, with the chemical name 5-[[4-[(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Bevacisumab which is commercially available as AVASTIN® is a humanized monoclonal antibody that blocks VEGF-A. AVASTIN® is approved form the treatment of various cancers including colorectal, lung, breast, kidney, and glioblastomas.

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Everolimus is sold as Afinitor® by Novartis and is the 40-O-(2-hydroxyethyl) derivative of sirolimus and works similarly to sirolimus as an mTOR (mammalian target of rapamycin) inhibitor. It is currently used as an immunosuppressant to prevent rejection of organ transplants and treatment of renal cell cancer. Much research has also been conducted on everolimus and other mTOR inhibitors for use in a number of cancers. It has the following chemical structure (formula V) and chemical name:

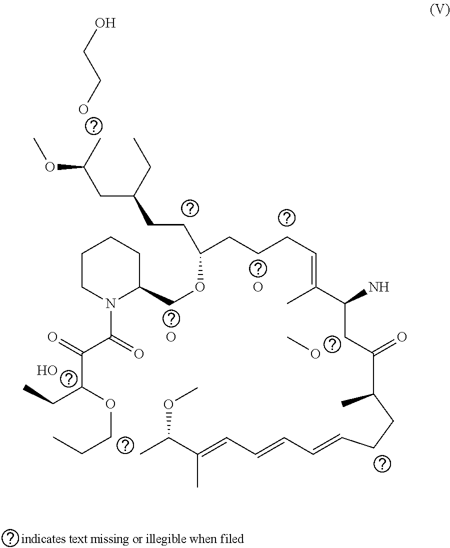

dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl] benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino]phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Agents used in immunotherapeutic regimens may also be useful in combination with the EZH2 inhibitors disclosed herein such as compounds of formula (I)-(IV), Compound B, or Compound C. There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by Formula VI, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

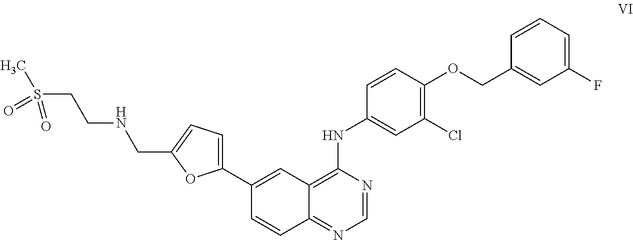 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (VI) may be prepared according to the procedures disclosed in WO 99/35146, published Jul. 15, 1999; and WO 02/02552 published Jan. 10, 2002.

Erlotinib, N-(3-ethynylphenyl)-6,7-bis {[2-(methyloxy)ethyl]oxy}-4-quinazolinamine Commercially available under the tradename Tarceva) is represented by formula VII, as illustrated:

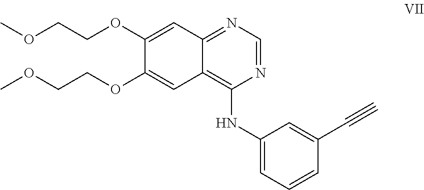 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine,N-(3-chloro-4-fluorophenyl)-7-methoxy-6-[3-4-morpholin)propoxy] is represented by formula VIII, as illustrated:

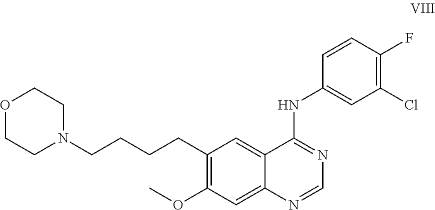 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

Trastuzumab (HEREPTIN®) is a humanized monoclonal antibody that binds to the HER2 receptor. It original indication is HER2 positive breast cancer.

Cetuximab (ERBITUX®) is a chimeric mouse human antibody that inhibits epidermal growth factor receptor (EGFR).

Pertuzumab (also called 2C4, trade name Omnitarg) is a monoclonal antibody. The first of its class in a line of agents called “HER dimerization inhibitors”. By binding to HER2, it inhibits the dimerization of HER2 with other HER receptors, which is hypothesized to result in slowed tumor growth. Pertuzumab is described in WO01/00245 published Jan. 4, 2001.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

Any of the cancer treatment methods of the claimed invention may further comprise treatment with at least one additional anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors if one of a mutation in EZH2 at Y641 or A677 or an increased level of H3K27me3 is detected.

### EXAMPLES

### Example 1: Tissue Culture

All cells were grown in RPMI 1640 (Invitrogen) supplemented with 10% heat-inactivated FBS and 1% penicillin/streptomycin.

### Example 2: Protein Extraction and Immunoblotting

Nuclear proteins were extracted using the Nuclear Complex Co-IP Kit (Active Motif). Proteins were electrophoretically separated, blotted and detected using enhanced chemiluminescence. Primary antibodies used were: H3K36me2 (Millipore 07-369), H3K27me3 (Millipore 07-449), MMSET[12] and pan-H4 (Abcam Ab7311). The secondary antibody used was horseradish peroxidase-conjugated donkey anti-rabbit IgG (GE Healthcare Life Sciences).

### Example 3: Chromatin Immunoprecipitation

ChIP experiments for histone modifications and MMSET were performed as described previously [12] using antibodies for H3K36me2 (Millipore, 07-369), H3K36me3 (Abcam, ab9050), H3K27me3 (Millipore, 07-449), MMSET[12], and rabbit IgG (Abcam, ab37415) as a negative control. Histone antibody specificity was confirmed using a MODified™ Histone Peptide Array (Active Motif), according to the manufacturer's instructions. JAM2 promoter primers were previously described [12]. EZH2 ChIP experiments (Cell Signaling, 5246s), were performed with following modification—cells were resuspended in nuclei lysis buffer (10 mM Tris pH 7.5, 10 mM NaCl, 0.2% NP-40, protease inhibitors) for ten minutes, centrifuged, washed and resuspended in SZAK RIPA buffer (150 mM NaCl, 1% v/v Nonidet P-40, 0.5% w/v deoxycholate, 0.1% w/v SDS, 50 mM Tris pH8, 5 mM EDTA, 0.5 mM PMSF, protease inhibitors) for sonication. Preparation of ChIP libraries and sequencing was performed by the Epigenomics Core at Weill Cornell Medical College. 10 ng of input and ChIP material was processed using the Illumina kit (IP-102-1001). Libraries were loaded onto a HiSeq 2000 or GAIIX at 6 pM, and subjected to 50 or 36 sequencing cycles, respectively. For histone modifications, data from the HiSeq 2000 is presented in the main text and experimental repeats from GAIIAX sequencing are presented in the supplemental figures. Both EZH2 ChIP-seq experiments were sequenced on HiSeq 2000. Raw reads were aligned to hg18 using ELAND or hg19 using Bowtie.

### Example 4: Histone ChIP-Seq Data Analysis

Several reads mapping to the same exact location were considered amplification artifacts and were excluded from the analysis. To analyze methylation patterns across the gene body, each gene body was divided evenly into 50 bins regardless of gene length. The immediate upstream and downstream 10 kb regions were also divided into 50 bins of 200 bp. Tag counts were normalized at each position by the average tag frequency per base pair of effective genome size. To compare the tag density distribution between different samples, tag density was normalized by the total number of the tags in each sample. To analyze the methylation pattern in the intergenic regions, the distance of the gene closest to the TSS was determined for each of the 15,386 genes. If the distance was at least 30 kb, this was selected as an intergenic region. The first and last 10 kb of each intergenic region was not included and the remaining region was divided evenly into 100 bins and analyzed as above.

### Example 5: EZH2 ChIP-Seq Data Analysis

Several reads mapping to the same exact location were considered amplification artifacts and were excluded from the analysis. Each ChIP-seq data set was normalized to its corresponding input lane. ChIP-seq peak calling, genomic annotation of peaks, target genes and comparison of EZH2 peaks in TKO and NTKO cells were performed using ChIPseeqer [64]. The default parameters were used for peak detection (i.e., 2-fold difference between ChIP and INPUT signal, and 10⁻¹⁵ statistical significance of the detected peaks). False discovery rates (FDR) for TKO samples were 0.08 and 0.02 for run 1 and run 2, respectively. For NTKO samples, FDR was 0.008 for run 1 and 0.003 for run 2. For both NTKO and TKO samples run 2 peaks were used for downstream analysis. Transcription factor motif analysis was performed using FIRE [65], included in ChiPseeqer, and HOMER [66]. Pathway analysis was performed using iPAGE [67], included in ChIPseeqer.

### Example 6: Microarray Analysis

Six replicate samples of both NTKO and TKO cells were run on an Illumina microarray (HumanWG-6_V3_0_R0_11282955_A). Among the 48,804 probes on the gene expression array, 28,124 probes were selected for further analysis based on having at least four positive expression values among the six replicates in both NTKO and TKO samples. Based on the Illumina annotation file (http://www.switchtoi.com/annotationfiles.ilmn), a final set of 15,386 protein-coding genes out of the 28,124 selected probes was identified. For each gene, a two-sample t-test was applied to obtain the p-value for significance of differential expression between NTKO and TKO cells. If a gene's expression level (in log scale) was lower than 3 in both NTKO and TKO cells, it was considered as a “not expressed” gene. A two-sample t-test was conducted for each gene not classified as “not expressed”. Genes detected as differentially expressed (p<0.002) were defined up or down modulated according to the sign oft-statistics. All other genes were classified as “not changed” in expression.

### Example 7: Gene Set Enrichment Analysis (GSEA)

GSEA 2.0 with default parameters was used to identify the enrichment of previously defined signatures among genes upregulated in TKO cells.

### Example 8: RNA Extraction, cDNA Synthesis, and qRT-PCR

RNA was extracted from cells using the RNeasy Plus Mini Kit (Qiagen). cDNA was synthesized from total RNA using the iScript™ cDNA Synthesis Kit (Bio-Rad). Quantitative RT-PCR was performed using predesigned TaqMan® assays (Applied Biosystems) for JAM2 (Hs00221894_m1), JAM3 (Hs00230289_m1), DLL4 (Hs00184092_m1), CA2 (Hs00163869_m1), CR2 (Hs00153398_m1), CDCA7 (Hs00230589_m1), LTB (Hs00242739_m1) and normalized to a GAPDH (Hs99999905_m1) control. qRT-PCR was run on a LightCycler® 480II (Roche).

### Example 9: EZH2 Inhibitor Treatment

1×10⁵ KMS11 and TKO cell lines were plated in the presence of 1 μM or 2 μM of GSK343 or GSK669 as a control. After seven days, cells were counted and proteins extracted for immunoblotting.

### Example 10: Cloning and Site-Directed Mutagenensis

All of the MMSET constructs were cloned into the pRetroX-DsRed vector (Clontech) except PHD1-M2 and PHD2-M2, which were cloned into pRetroX-ZsGreen (Clontech). A nuclear localization signal was inserted at the N-terminus of PHD1-M2 and PHD2-M2 constructs. Site-directed mutagenesis of PHD fingers 2 and 3 was performed using QuickChange Lighting Site-Directed Mutagenesis Kit (Stratagene) following the manufacturer's recommendations.

### Example 11: Infection

For the repletion system, TKO cells were transduced with retroviral vectors harboring MMSET or mutant isoforms. All retroviruses were produced by transfection of amphotropic 293T cells with appropriate plasmids and FuGENE 6 Transfection reagent (Roche). After infection, cells were sorted by flow cytometry using the DsRed protein marker and expanded in culture for further studies.

### Example 12: Colony Formation Assay

2×10³ cells were grown in 1 mL of semisolid methylcellulose medium (MethoCult H4100; StemCell Technologies Inc.) supplemented with complete medium and heat-inactivated FBS. Two weeks later, colonies were counted in at least 6 random fields.

### Example 13: Cell Growth

3×10⁴ cells were grown in a 6-well plate with 2 mL of complete medium. Live cells were collected and counted at indicated days using trypan blue dye.

### Example 14: Mouse Xenograft Model

Six-week-old female C57BL6 Nu/Nu mice were obtained from The Jackson Laboratory and were acclimated for at least for 24 h before tumor cell injection. A total of 5×10⁶ KMS11 cells harboring an inducible MMSET shRNA were resuspended in 100 μL cold PBS and were mixed with 100 μL of CultreX PathClear BME (3432-005-02, Trevigen). The mixture was injected subcutaneously in the dorsal region next to both thighs. One week after injection, mice were divided in two groups (n=5 per group). The control group was administered regular water and the treatment group was given doxycycline 2 mg/mL in water containing 0.04 g/mL of sucrose. The water was changed every other day to ensure delivery of a stable dose of doxycycline. Two weeks after treatment initiation, images were acquired using IVIS^{R} Spectrum (Caliper Life Sciences, Inc.). For imaging, firefly Luciferin (150 mg/kg) (Gold Biotechnology) was injected intraperitoneally and images were taken 10-15 min later. Bioluminiscence was quantified using Living Images software (Caliper Life Sciences, Inc.). GraphPad Prism software was used for survival analysis.

For protein extraction, tumor samples were immediately frozen in liquid nitrogen and stored at −80° C. Frozen tumors were mechanically homogenized using a biopulverizer (Biospec) chilled at −80° C., and incubated in lysis buffer (10 mM Hepes ph 7.9, 10 mM KCl, 1.5 mM MgCl₂, 0.5% NP40, 1 mM PMSF, 1 mM DTT, proteinase inhibitors) on ice for 20 min. Upon centrifugation, the supernatant containing the cytoplasmic fraction was discarded and nuclei were resuspended in lysis buffer containing 20 mM Hepes pH 7.9, 400 mM NaCl, 1.5 mM MgCl₂, 0.2 mM EDTA, 15% glycerol, 1 mM PMSF, 1 mM DTT and proteinase inhibitors. Lysates were incubated at 4° C. for 20 min on an orbital rotator and further sonicated for 20 min using a Bioruptor (Diagenode, Inc) (30 seconds on, 30 seconds off). The supernatant containing nuclear proteins was analyzed by immunoblot.

### Example 15: MMSET Alters the Epigenetic Landscape of t(4;14)+ Myeloma Cells

We and others have reported that the overexpression of MMSET in t(4; 14)+ myeloma cells increases global levels of H3K36 dimethylation [11, 12]. To investigate how the pattern of H3K36me2 genomic distribution is affected by MMSET abundance, we performed chromatin immunoprecipitation followed by high-throughput sequencing (ChIP-seq) in TKO and NTKO cells (two independent biological replicates for each sample). TKO is a t(4;14)+ KMS11 cell line in which the rearranged IGH-MMSET allele has been inactivated by homologous recombination. These cells express only one wild-type copy of the MMSET gene at physiological levels yielding low basal levels of H3K36me2 (FIG. 1A) [25]. In their counterpart, NTKO cells, the wild-type MMSET allele is inactivated, but high levels of MMSET and H3K36me2 are maintained from the remaining rearranged IGH-MMSET allele (FIG. 1A). We performed microarray analysis in NTKO and TKO cells to obtain global expression levels in these cells. In agreement with previous studies [11], in MMSET-low TKO cells, the presence of H3K36me2 positively correlated with highly expressed genes (FIG. 1B; Supplemental FIGS. S1A, S1B). Typically, the enrichment of H3K36me2 peaked just upstream of the transcription start site (TSS) and decreased towards the 3′ end of a gene (FIGS. 1B, 1C; FIG. 8B). Our ChIP-seq analysis of NTKO cells revealed that this characteristic distribution of H3K36me2 was disrupted in MMSET-high conditions. Despite very high global levels of H3K36 dimethylation in NTKO cells, H3K36me2 enrichment did not localize to specific loci or domains (FIGS. 1B and 1C intragenic). Instead, H3K36me2 enrichment was dispersed more evenly throughout the genome and lacked clear boundaries (FIG. 1C intergenic, 1D bottom). In MMSET-high NTKO cells characteristic peaks of H3K36me2 adjacent to the TSS were eliminated and a lower uniform level of H3K36me2 was measured throughout gene bodies (FIG. 1C intragenic; FIG. 8B). This decrease of intragenic H3K36me2 seemed paradoxical as immunoblot and mass spectrometry clearly demonstrated an approximately 8-fold increase of H3K36me2 in NTKO cells (FIG. 1A) [12, 26]. This inconsistency was resolved by examining H3K36me2 within intergenic regions, which comprise 97% of the genome compared to the 3% of the genome that is protein-coding [27]. Plotting the density of sequence tags across 6,172 intergenic regions and comparing the relative H3K36me2 enrichment revealed that in MMSET-high NTKO cells there is a ˜3-fold increase in abundance of the H3K36me2 modification (FIG. 1C). A large scale view over a gene-rich region illustrates that peaks of H3K36me2 are obliterated in MMSET-high NTKO cells (FIG. 1D, top), whereas generally low levels of H3K36me2 in a gene-poor region are increased in the presence of high levels of MMSET (FIG. 1D, bottom; FIG. 8B).
Despite the wide-ranging increase of H3K36me2 levels in NTKO cells, we found that MMSET affects expression of only a specific subset of genes. Gene expression profiling identified 522 genes upregulated with overexpression of MMSET and 308 genes that are repressed in MMSET-high NTKO cells (FIG. 2A; Supplemental Data S1). To better understand the basis of expression changes associated with MMSET abundance, we examined the compiled H3K36me2 ChIP-seq profiles of genes sorted based on their expression pattern in NTKO and TKO cells. Specifically, regions upstream of the TSS of genes activated in the presence of MMSET are more enriched for H3K36me2 in NTKO than TKO cells (FIG. 2B, red line). These include adhesion molecules such as JAM2 and JAM3 as well as CR2 (FIG. 2C), the latter of which was shown to play a role in the interaction of myeloma cells with the bone marrow stroma [28]. However, within gene bodies of genes activated by MMSET, the levels of H3K36me2 were very comparable between NTKO and TKO cells (FIG. 2B, red line). This result suggested that the action of MMSET at the promoters was important in regulation of these genes. Genes that were not expressed in either NTKO or TKO cells did not have any significant changes in the density of H3K36me2 (FIG. 2B, light blue line). However, genes repressed in the presence of MMSET (FIG. 2B, green line) or genes whose expression is not altered due to MMSET (FIG. 2B, dark blue line) seemed to be protected from the global increase in H3K36me2 in NTKO cells and were found to have higher levels of H3K36me2 in MMSET-low TKO cells. We also performed ChIP-seq for trimethylated H3K36 (H3K36me3) in NTKO and TKO cells, a chromatin mark enriched in gene bodies of highly expressed genes [29]. Although global H3K36me3 distribution patterns were similar between NTKO and TKO cells (FIG. 2D; FIG. 9), as expected, the subset of genes upregulated in MMSET-high NTKO cells exhibited elevated enrichment of H3K36me3 throughout their intragenic regions (FIG. 2E). Similarly, genes downregulated in the presence of high levels of MMSET, such as DLL4, correlated with increased levels of H3K36me2 and H3K36me3 in MMSET-low TKO cells (FIGS. 2C and 2E).
The relatively high number of repressed genes in NTKO cells was unexpected given the global chromatin effects, increased H3K36me2 and decreased H3K27me3, associated with MMSET overexpression (FIG. 1A). ChIP-seq analysis of H3K27me3 in NTKO and TKO cells revealed that the upregulation of gene expression in the presence of MMSET was accompanied by a loss of H3K27me3, particularly in regions 5′ to the TSS (FIGS. 3A, red and 3B). Gene Set Enrichment Analysis (GSEA) showed that the subset of genes upregulated in MMSET-high NTKO cells represent direct targets of EZH2 and H3K27 methylation [30-32]. This suggests that MMSET itself or its resultant H3K36me2 modification may prevent PRC2 binding to these loci, thereby inducing gene expression by relief of active Polycomb repression (FIG. 3C). By contrast, genes with expression levels that were unaffected by MMSET abundance showed no significant difference in H3K27me3 enrichment despite the global decrease of H3K27me3 in NTKO cells (FIG. 3A, dark blue). However, genes repressed in the presence of high levels of MMSET or transcriptionally silent in both cell types showed an increased abundance of H3K27me3 in MMSET-high NTKO cells (FIG. 3A, green, light blue). On many genes repressed in the presence of MMSET a large increase of H3K27me3 near the start site of transcription was observed in NTKO cells (FIG. 3D). The HOXC cluster, whose expression could not be detected in this cell system, demonstrated fairly high levels of H3K27me3 dispersed across a >60 kb genomic segment in MMSET-low TKO cells, that was increased ˜2-fold in MMSET-high NTKO cells (FIG. 3E). The chromatin landscape of genes exhibiting decreased expression in MMSET-high cells was examined in TKO cells. Here, genes that were repressed in the presence of MMSET and activated in TKO cells had increased levels of H3K36me2 and H3K36me3 and decreased levels of H3K27me3 (FIG. 3F), and included known EZH2/PRC2 targets, such as DLL4 and CDCA7 (FIG. 3D) [31, 33]. Thus, overexpression of MMSET not only modifies H3K36me2 distribution, but also leads to a drastic change in the distribution pattern of the repressive H3K27me3 mark. Whereas MMSET-overexpressing myeloma cells show a genome-wide decrease in H3K27me3, specific loci are able to maintain and even gain higher levels of H3K27 methylation in the presence of MMSET, leading to transcriptional repression.

### Example 16: MMSET Overexpression Alters EZH2 Binding

Our previous work demonstrated that the epigenetic landscape in t(4;14)+ cells is established by a competition between the methylation activities of EZH2 and MMSET for the histone H3 tail substrate [26]. Specifically, active chromatin marks, including H3K36me2, were shown to inhibit PRC2 from both binding the nucleosomes and methylating the histones [34, 35]. Kinetic studies revealed that once histone H3 reaches the dimethylation state at H3K36, the effective rates of H3K27 di- and trimethylation on the same histone molecule drop dramatically [26]. Furthermore, we showed that MMSET-high cells have increased rates of H3K27 demethylation contributing to the global loss of this modification [26]. These data suggest that the global decrease of H3K27 methylation in the presence of MMSET may be due to the inability of EZH2 and the PRC2 complex to bind chromatin. If this is true, then an increased concentration of unbound EZH2 would be available to bind genomic regions that are able to maintain H3K27me3 in the presence of high levels of MMSET. Therefore, this increased ratio of free enzyme to available substrate could be responsible for the enhanced enrichment of H3K27 trimethylation at specific loci in NTKO cells.
To test this hypothesis, we examined EZH2 distribution in TKO and NTKO cells by ChIP-seq (two highly correlated independent biological replicates, r=0.82 and r=0.84, respectively) (FIG. 10A). This analysis identified 10,581 EZH2 peaks (associated with the promoters of 1,697 genes) in the MMSET-high NTKO cells and 5,516 (953 genes) in the TKO cells (FIG. 4A). Of these genes, 733 were common between the two cell types and 964 genes had enriched EZH2 binding exclusively in the NTKO cells. As predicted, EZH2 peaks that are shared between NTKO and TKO cells are on average higher and broader in NTKO cells (FIG. 10B). In MMSET-high cells, enhanced localization of EZH2 closely tracked with H3K27me3 enrichment (FIG. 4B), including DLL4 and CDCA7 promoters (FIG. 4C). Analysis of genes bound by EZH2 only in MMSET-high NTKO cells, using a library of lymphoid biology gene expression signatures [36], showed that they included genes known to play a role in normal germinal center B cells (GC_B_cell category), as well as known B cell MYC targets (Myc_ChIP category) (FIG. 4D and Supplemental Data S2). Thus, aberrant EZH2-mediated repression of genes known to play a role in lymphoid biology may be important for MMSET-induced oncogenesis. Loci bound by EZH2 only in TKO cells were enriched for genes found to be upregulated in t(4;14)+ patient samples (FIG. 4D, Myeloma_MS category), suggesting that MMSET overexpression reverses normal silencing of these genes by the PRC2 complex.
Considering that many EZH2 bound regions were unique to either MMSET-high or MMSET-low cells, we examined the underlying sequence to determine if specific transcription factor motifs were over-represented within these regions. This analysis revealed that regions bound by EZH2 exclusively in MMSET-low TKO cells coincided with GATA3, HOXA2 and PDX1 motifs (FIG. 4E). In breast cancer cells, GATA3 and EZH2 are functionally antagonistic, suggesting that similar interplay between these two factors may also exist in myeloma [37]. Interestingly, EZH2-bound regions specific to MMSET-high NTKO cells were associated with DNA motifs that resemble known CTCF DNA binding sites (FIG. 4E), implying a possible mechanism where insulator sequences may protect these loci from methylation by MMSET. Additional DNA motifs included poly-G and poly-C-rich sequences (FIG. 11C), resembling PRC2 recruitment motifs defined in ES cells [38]. To determine whether the enhanced binding of EZH2 may play a functional role in myeloma cell survival, we treated MMSET-high and MMSET-low cells with recently described small molecule inhibitor of EZH2 [39]. Indeed, MMSET-high cells were more sensitive to EZH2 inhibition (FIG. 4F and FIG. 10D), suggesting that some of the newly acquired EZH2 binding sites in MMSET-high cells are critical for survival of these cells. Together, we conclude that MMSET overexpression alters the genomic organization of EZH2 across the myeloma genome and this effect, similar to other cancers, induces misregulation of specific Polycomb target genes that contribute to pathogenesis.

### Example 17: Oncogenic MMSET Function Depends on its PHD and PWWP Domains

In addition to the enzymatically active SET domain, MMSET possesses four PHD domains commonly implicated in chromatin binding [40]. To determine whether these and other conserved domains of MMSET are required for myelomagenesis, we repleted TKO cells with either wild-type MMSET or deletion mutants and assessed for changes in chromatin modifications, gene expression and growth (FIG. 5A). Expression of wild-type MMSET in TKO cells re-established high levels of H3K36me2 and loss of H3K27me3 (FIG. 5B), activated transcription of specific genes, such as JAM2 (FIG. 5C and FIG. 11A), stimulated proliferation (FIG. 11B) and increased colony formation (FIG. 5D; FIG. 11C). A point mutation at tyrosine 1118 (Y1118A) that abrogates the HMT activity of MMSET [12] prevented the re-establishment of H3K36 and H3K27 methylation in vivo (FIG. 5B; FIG. 11D) and failed to stimulate gene expression (FIG. 5C), cell growth [12] and colony formation (FIG. 5D and FIG. 11C). A construct missing the C-terminal portion of the protein, including PHD finger #4 (−PHD4), was able to methylate H3K36, albeit at lower levels (FIG. 5B) [26], and resulted in an incomplete loss of H3K27 methylation (FIG. 5B), yielding an intermediate alteration of gene expression (FIG. 5C; FIG. 11A), growth stimulation (FIG. 11B) and colony formation (FIG. 5D and FIG. 11B). These data suggest that the biological contribution of MMSET in myeloma cells not only depends on its ability to stimulate H3K36me2 levels, but also depends on the degree of inhibition of H3K27 methylation.
MMSET also contains two PWWP domains, the first of which has been suggested to nonspecifically bind chromatin [41]. However, in many MM cases, the t(4;14) breakpoint disrupts MMSET 3′ to the exons encoding PWWP1, leading to the overexpression of a truncated MMSET lacking this domain. Thus, while PWWP1 may play an important role for normal MMSET function, it likely does not contribute to oncogenesis. Indeed, overexpression of an MMSET construct missing the N-terminal region of the protein, including the first PWWP domain, led to the same epigenetic switch observed with overexpression of the wild-type protein (data not shown). However, loss of the second PWWP domain rendered MMSET enzymatically inactive (FIG. 5B), and this construct (−PWWP2) was unable to stimulate growth (FIG. S4E), alter gene expression (FIG. 5C; FIG. 11A) or promote colony formation (FIG. 5D; FIG. 11C). Interestingly, all deleted or mutated constructs were still able to bind chromatin (FIG. 5E). Nevertheless, both −PWWP2 and Y1118A mutants were unable to mediate methylation of lysine H3K36 on the JAM2 locus and thus are unable to induce gene expression (FIG. 5C; FIG. 11A). By contrast, −PHD4 expression led to H3K36 methylation but its inability to induce complete demethylation of H3K27 allowed for only partial JAM2 activation (FIG. 5C; FIG. 11A). We conclude that the ability of MMSET to induce a complete H3K36/H3K27 methylation switch in myeloma cells depends on a complex interplay of several domains of the protein. Furthermore, our data suggest that both methylation of H3K36me2 and demethylation of H3K27me3 are required for MMSET to fully alter gene expression observed in myeloma.

### Example 18: MMSET Binding to Chromatin and Methylation of Histones Depends on Specific PHD Domains

We showed previously that the MMSET C-terminal isoform, REIIBP, which contains the third and fourth PHD fingers, the second PWWP domain and the SET domain, is not able to methylate histones in TKO cells [12]. We systematically added back additional domains of MMSET to REIIBP and found that addition of PHD finger 2 or PHD fingers 1 and 2 together (FIG. 6A) induced methylation of H3K36 and demethylation of H3K27me3 (FIG. 6B), as well as enhanced colony formation (FIG. 12A). Mutations and deletions in NSD1, an HMT closely related to MMSET, are implicated in Sotos syndrome, a disorder characterized by developmental overgrowth and cognitive disabilities [42]. We mapped previously identified mutations in NSD1 from Sotos syndrome patient samples to MMSET in attempt to identify important domains that are required for proper function of the two proteins (FIG. 6A). Single point mutations of cysteine residues 720, 735 or 857, all within the second or third PHD finger of MMSET, rendered MMSET incapable of modulating H3K36 and H3K27 levels (FIG. 6C). Similar to the enzymatically-dead SET domain mutant, expression of these PHD point mutants in TKO cells failed to stimulate colony formation (FIG. 6D; FIG. 12B) or activate gene expression (FIG. 6E). Importantly, ChIP assays for MMSET in repleted TKO cells revealed that the C720R and C857R mutant proteins exhibited dramatically reduced binding to the JAM2 promoter, likely explaining their failure to methylate histones (FIG. 6F). These data suggest that the PHD fingers of MMSET play an important role in recruitment of the protein to chromatin. In addition, these findings suggest that the Sotos syndrome mutations in NSD1 may have similar consequences, rendering the enzyme incapable of regulating chromatin structure and gene expression.

### Example 19: Targeting MMSET Decreases Tumor Burden in NOD/SCID Mice

Elevated expression of MMSET in a number of different types of cancer suggests that inhibiting MMSET may be therapeutically advantageous beyond multiple myeloma. However, because MMSET translocation in myeloma occurs early in the premalignant MGUS (Monoclonal Gammopathy of Undetermined Significance) stage of the disease, it is unclear to what extent fully developed tumors depend on MMSET expression or whether targeting MMSET can lead to tumor reduction. To test whether MMSET reduction can inhibit myeloma growth in vivo, we injected the flanks of NOD/SCID mice with t(4;14)+ KMS11 cells expressing a doxycycline-inducible shRNA targeting MMSET. We previously demonstrated that expression of this shRNA decreases MMSET and H3K36me2 levels, increases H3K27me3 levels and leads to cell growth arrest [12]. Expression of the luciferin gene in the KMS11 cells allowed for in vivo live-cell imaging to monitor disease development. Tumors were allowed to grow for seven days, after which half of the mice were given doxycycline (dox) in their water to induce shRNA expression. As a result, all of the treated animals had dramatically reduced tumor volumes and in some cases, complete regression (FIGS. 7A-C and FIG. 13). By contrast, five weeks after injection of tumor cells, all untreated animals required sacrifice due to tumor progression. The reduction in tumor size in dox-treated animals was accompanied with a global loss of H3K36 dimethylation and an increase in H3K27 trimethylation (FIG. 7D). To determine whether this was a long-lasting effect, we removed doxycycline after four weeks of treatment and observed the animals for four additional weeks. Even in the absence of shRNA expression, some tumors continued to decrease in size (FIG. 7A). Animals whose tumors disappeared completely remained tumor-free even in the absence of doxycycline. However, tumors that persisted during induction of the MMSET shRNA eventually started to grow back upon doxycycline removal, albeit at a reduced rate. Thus, we conclude that established t(4;14)+ tumors depend on MMSET expression for their proliferation and that inhibition of MMSET function represents a rational form of therapy targeting against cancers that express high levels of this protein.

### Example 20: MMSET is a Global Epigenetic Regulator

Deregulation of epigenetic machinery is one of the main drivers of oncogenic transformation and cancer development. While alterations of many epigenetic regulators seem to affect a specific subset of downstream gene targets and pathways, there is a growing number of examples where deregulation of a single component of the machinery affects the global epigenetic landscape, including mutations in EZH2, TET2, ASXL1 and SETD2, among others [5, 7, 8, 43-45]. Besides affecting gene regulation, epigenetic anomalies that change overall chromatin structure might affect other chromatin-dependent processes such as DNA repair and DNA replication.
In t(4;14)+ myeloma, overexpression of MMSET induces a dramatic increase in H3K36 dimethylation throughout the genome. Normally, the H3K36me2 mark is enriched in the 5′ and 3′ proximity of the TSS of highly expressed genes. Increased methylation levels in the presence of MMSET alter the distribution of this mark, leading to a net decrease in many gene bodies and a significant increase in intergenic regions, with the result being an ˜8-fold overall increase in H3K36me2 levels [26]. The precise role of H3K36me2 in transcriptional regulation is still poorly understood and requires further investigation. However, our data show that the global increase in H3K36 methylation leads to a concomitant genome-wide decrease of H3K27 methylation. This result is in agreement with previous in vitro studies showing that activating histone marks, including H3K36 methylation, antagonize H3K27 methylation through prevention of PRC2 binding to chromatin [34]. Surprisingly, our analysis of H3K27me3 patterns in the presence of high levels of MMSET show that while most of the genome is hypomethylated at this residue due to increased H3K36me2, specific loci, including previously identified Polycomb targets, are hypermethylated on lysine 27 through enhanced recruitment of EZH2. A study by Kalushkova et al. showed that Polycomb targets are normally silenced in multiple myeloma cells and our study identifies one possible mechanism explaining how this may be achieved [46].
EZH2/PRC2 complexes are recruited to chromatin via sequence-specific transcription factors [47], through the ability of PRC2 component Jarid2 to bind to DNA [48] and through the ability of the EZH2 accessory protein EED to recognize and bind to the H3K27me3 mark [49]. High levels of MMSET in the t(4;14)+ cells lead to an increased rate of H3K36 methylation, precluding the action of EZH2 and removing potential chromatin binding sites for the PRC2 complex. EZH2 and PRC2 component levels do not change in MMSET-high NTKO cells and thus we propose a model where the PRC2 complex in the nucleus is displaced from many genomic sites (FIG. 7E). However, certain loci fail to become hypermethylated on H3K36me2 in MMSET-high cells. Among those, some sites have modest levels of H3K27me3 and EZH2 binding in MMSET-low cells that are further enhanced in MMSET-high cells, while other loci only accumulate appreciable levels of EZH2 and H3K27 in the presence of high levels of MMSET. Many of the EZH2 peaks enhanced and unique to MMSET-high cells sit on CTCF sites, known insulators that block the spread of chromatin marks. We propose that the inability of MMSET to methylate CTCF-bound sites prevents the global spreading of H3K36me2 to these regions, allowing EZH2 to be retained or recruited to such loci. Previous studies in Drosophila showed that genome-wide binding of CTCF aligns with H3K27me3 domains [50]. Additionally, our model is in agreement with recent work from Gaydos et al. showing that in C. elegans, H3K36 methylation by MMSET homologue MES-4 antagonizes H3K27 methylation across autosomes and concentrates H3K27me3 on the X chromosome [51]. Loss of MES-4 expression allows for spreading of the H3K27me3 mark on autosomes and concomitant loss of the mark on the X chromosome. Our findings, as well as those by Gaydos et al., argue that localization of the H3K27 methyl mark greatly depends on the number of genomic loci that are accessible for PRC2 activity. Our identification of CG-rich DNA motifs at sites of enhanced EZH2 enrichment in MMSET-high cells, similar to those previously described to aid in recruitment of EZH2 to chromatin [38], suggests that the underlying DNA sequence also plays a role in specifying genes particularly responsive to EZH2 activity. Furthermore, CpG islands have been shown to recruit KDM2A, an H3K36-specific demethylase, which may provide a suitable, H3K36-demethylated chromatin template for PRC2 binding [52]. Recently, a recurrent mutation in the gene encoding histone H3 isoform H3.3 was identified in pediatric glioblastoma patients, which converts lysine 27 to methionine [53]. In addition to a genome-wide decrease in H3K27 methylation, as in the case of MMSET overexpression, the H3K27M mutation also induces focal increases in EZH2 and H3K27 methylation and aberrant gene repression. Thus, the mechanism suggested by our study may be applicable to other malignancies characterized by disrupted H3K27 methylation.

### Example 21: MMSET as a Therapeutic Target

Multiple studies indicate that high levels of MMSET are not exclusive to t(4;14)+ myeloma. Overexpression of MMSET also occurs in a number of solid tumors [20, 21] and is correlated with the stage and aggressiveness of the disease. In prostate cancer, the upregulation of EZH2 in high grade and metastatic disease represses miR-203, which targets MMSET, explaining, at least in part, MMSET upregulation [54]. Perhaps due to the parallel increase of MMSET and EZH2 in prostate and other tumors, studies to date have not shown a net increase in H3K36 or depression of H3K27me3 in advanced-stage cancers. Nevertheless, we showed that siRNA depletion of MMSET in metastatic but not in non-transformed prostatic epithelial cells results in a switch in H3K36/H3K27 methylation, suggesting that metastatic cancer cells may have increased dependency on MMSET for lysine 36 methylation [20]. Additionally, we recently showed that in acute lymphoblastic leukemia, a recurrent mutation within the SET domain of MMSET enhances its methyltransferase activity and induces a global epigenetic change similar to what is observed when MMSET is overexpressed [24].
We and others showed that the histone methyltransferase activity of MMSET is key to its oncogenic potential [11, 12]. However, the full “chromatin switch” driven by MMSET overexpression also depends on the second PWWP domain and the PHD fingers 2, 3 and 4 (FIGS. 5 and 6). Loss of the second PWWP domain leads to recruitment to chromatin but failure to methylate H3K36. This effect might be due to allosteric interactions between functional domains or improper alignment of the protein on the nucleosome or DNA. In support of this idea, Li et al. showed that in vitro methylation activity of MMSET was augmented by the addition of DNA to the reaction mixture or by the use of nucleosomes as a substrate [10]. The loss of the fourth PHD domain is particularly interesting, as this truncation yields an intermediate biological phenotype with an incomplete loss of H3K27 methylation even in the presence of a global increase in H3K36me2. This region of MMSET was shown to have an affinity for unmethylated histone H3 peptides in vitro, but its deletion, unlike the deletion of the PWWP domain, did not block its ability to methylate chromatin. Instead, the resulting partial switch in chromatin was associated with incomplete gene activation and modest growth stimulation, highlighting the importance of H3K27me3/EZH2 dysfunction in the biology of MMSET. The mechanism by which deletion of PHD4 prevents loss of H3K27me3 remains unexplained. The enhanced rate of H3K27me3 demethylation we observed in MMSET-high NTKO cells [26] suggests that MMSET may affect the activity of H3K27me3 demethylases. Alternatively, the genome-wide distribution or effects of MMSET on H3K36me2 may be qualitatively different with the loss of a domain that attracts MMSET to chromatin. NSD1, a close homologue of MMSET, is fused to the NUP98 locus in rare cases of acute myeloid leukemia creating the NUP98-NSD1 fusion protein [55]. Interestingly, the ability of NUP98-NSD1 to transform mouse bone marrow cells and to activate Hox gene expression depends on the presence of the analogous fourth PHD finger of the NSD1 moiety [56]. The other PHD domains of MMSET are also critical for its oncogenic function. This was demonstrated by engineering mutations into PHD fingers 2 and 3 analogous to those found in NSD1 in Sotos syndrome patients [57]. These point mutations of MMSET failed to bind chromatin and failed to alter chromatin methylation. Our findings indicate that PHD domains are additional regions of MMSET that may be considered as therapeutic targets and suggest how these point mutations may inactivate NSD1 in Sotos syndrome.
The sequencing of the coding regions and genomes of a variety of human tumors showed that mutations in the epigenetic apparatus are among the most common class of alterations in cancer [43, 45, 58], further stimulating interest in epigenetically targeted therapies [1]. While germinal cell lymphoma is associated with gain-of-function mutations of EZH2 [5], multiple myeloma has not been linked directly to alterations in EZH2 function. However, mutations in the H3K27me3 demethylase UTX are found in 30% of myeloma cases [59]. While the role of UTXmutations in myelomagenesis is still unclear, it likely involves increases in H3K27 methylation and aberrant gene repression. Thus, the focal increase of H3K27 methylation in the presence of MMSET may have a similar effect as UTX mutations, suggesting that EZH2 plays an important, and so far underappreciated, role in multiple myeloma. Prior work implicated EZH2 in myeloma cell proliferation and transformation [60], and our data suggest that t(4;14)+ cells may be particularly sensitive to inhibition of EZH2 (FIG. 4F). Similarly, MMSET is commonly misregulated in human cancers and inhibition of MMSET activity may have therapeutic potential for diverse tumors. In tumorigenic prostate cells, MMSET expression maintains the transformed phenotype by stimulating cell growth, migration and invasion [20, 54]. Inhibition of MMSET function in MM cells by shRNA in established xenografts led to tumor regression in association with reversal of the chromatin changes. Therefore, we hypothesize that agents that block the enzymatic activity of MMSET or its ability to properly dock with chromatin could represent potential new therapies. Although inhibition of enzymatic activities of proteins such as MMSET and EZH2 is a rational approach, recent success in targeting chromatin-reading domains of BRD4 suggest that inhibition of non-enzymatic domains should also be considered [61]. Indeed, our data suggest that targeting the PHD fingers or PWWP domain may be equally sufficient in preventing MMSET from methylating histones and altering gene expression. Recently identified inhibitors of EZH2 [62, 63] and hopefully soon to be identified inhibitors of MMSET will allow us to determine the therapeutic effects of these targeted therapies on a number of cancer subtypes, including patients with t(4;14) translocations.

### Example 22: EZH2 is a Novel Therapeutic Target in Myeloma Cells

Precise spatial and temporal gene expression is required for normal development and aberrant regulation of gene expression is a common factor in many diseases, including cancer. Histone modifications contribute to the control of gene expression by altering chromatin structure and affecting the recruitment of transcriptional regulators. In this study, we demonstrate interplay between two oncogenic proteins, MMSET and EZH2, known to methylate histone H3 on lysine 36 (H3K36) and lysine 27 (H3K27), respectively. Overexpression of MMSET in myeloma cells increases global levels of H3K36 methylation, alters its normal distribution throughout the genome and decreases global levels of H3K27 methylation. We found that while the majority of the genome loses H3K27 methylation in the presence of MMSET, certain loci have augmented recruitment of EZH2 and enhanced H3K27 methylation, leading to transcriptional repression. Repression of these genes likely plays an important role in the disease because MMSET-overexpressing cells show higher sensitivity to small molecule inhibitors targeting EZH2-mediated methylation. Thus, our study suggests that the specific local changes may outweigh the gross global changes we frequently observe in cancer and implicates EZH2 as a novel therapeutic target in myeloma cells.

### Example 22: MMSET and UTX in Multiple Myeloma Cell Lines

Cells were treated with the EZH2 inhibitors below. GSK2816126A is also known as GSK126 and is a compound of Formula I, specifically Compound B as described herein. Data in Table I was used to generate the data in FIG. 14.
EZH2 inhibitors of Formula II, III, and IV and Compound C are used to generate gIC50 values and similar results as with GSK126 are found.
Additional cell lines are tested with EZH2 inhibitors of Formula I, II, III, IV, Compound B, and Compound C and an improved cell killing, e.g. as measured by gIC50, is seen in cell lines having an increased level of MMSET expression, or a decreased level of UTX enzyme, or both, as compared to a control, e.g. a tissue typed control sample not characterized as having cancerous attributes.

TABLE I

| Cell | GSK3189726A | GSK3126034B | GSK2603343A | GSK2816126A |
|---|---|---|---|---|
| Line | gIC50 (nM) | gIC50 (nM) | gIC50 (nM) | gIC50 (nM) |
| ARH-77 | 36657 | 19400 | 11230 | not measured |
| U266B1 | 25413 | 32613 | 4087 | not measured |
| LP1 | 1949 | 6779 | 2256 | 2377 |
| KMS-11 | 895 | 736 | 3166 | 3015 |
| RPMI- | 225 | 467 | 6186 | 1685 |
| 8226 | | | | |
| KMS- | 182 | 983 | 4857 | 2568 |
| 12-BM | | | | |
| OpM2 | 70 | 229 | 5239 | 861 |
| L363 | 10 | 66 | 2725 | not measured |

TABLE II
Mutational status of the cell lines in Table I

| | | H3K36me2 | |
|---|---|---|---|
| Cell Line | MMSET (MTM) | levels | UTX |
| ARH-77 | none | not measured | wt |
| U266B1 | none | low | wt |
| LP1 | t(4; 14) +; MB4-2 | high | Hom 1621C > T (Q541X) |
| KMS-11 | t(4; 14) +; MB4-1 | high | not measured |
| RPMI- | none | low | wt |
| 8226 | | | |
| KMS-12- | none | not measured | Hom del exons 5-12 |
| BM | | | |
| OpM2 | t(4; 14) +; MB4-3 | high | Het c.197G > A p.G66D |
| L363 | none | low | Horn Del exons 1, 2 |

### REFERENCES, EACH OF WHICH IS INCORPORATED BY REFERENCE HEREIN. TO THE EXTENT THE DEFINITIONS CONFLICT, THIS DOCUMENT CONTROLS

1. Popovic R, Licht J D (2012) Emerging epigenetic targets and therapies in cancer medicine. Cancer Discov 2: 405-413.
2. Morey L, Helin K (2010) Polycomb group protein-mediated repression of transcription. Trends Biochem Sci 35: 323-332.
3. Vire E, Brenner C, Deplus R, Blanchon L, Fraga M, et al. (2006) The Polycomb group protein EZH2 directly controls DNA methylation. Nature 439: 871-874.
4. Boyer L A, Plath K, Zeitlinger J, Brambrink T, Medeiros L A, et al. (2006) Polycomb complexes repress developmental regulators in murine embryonic stem cells. Nature 441: 349-353.
5. Morin R D, Johnson N A, Severson T M, Mungall A J, An J, et al. (2010) Somatic mutations altering EZH2 (Tyr641) in follicular and diffuse large B-cell lymphomas of germinal-center origin. Nat Genet 42: 181-185.
6. Beguelin W, Popovic R, Teater M, Jiang Y, Bunting K L, et al. (2013) EZH2 is required for germinal center formation and somatic EZH2 mutations promote lymphoid transformation. Cancer Cell 23: 677-692.
7. Abdel-Wahab O, Adli M, LaFave L M, Gao J, Hricik T, et al. (2012) ASXL1 mutations promote myeloid transformation through loss of PRC2-mediated gene repression. Cancer Cell 22: 180-193.
8. Ernst T, Chase A J, Score J, Hidalgo-Curtis C E, Bryant C, et al. (2010) Inactivating mutations of the histone methyltransferase gene EZH2 in myeloid disorders. Nat Genet 42: 722-726.
9. Score J, Hidalgo-Curtis C, Jones A V, Winkelmann N, Skinner A, et al. (2012) Inactivation of polycomb repressive complex 2 components in myeloproliferative and myelodysplastic/myeloproliferative neoplasms. Blood 119: 1208-1213.
10. Li Y, Trojer P, Xu C F, Cheung P, Kuo A, et al. (2009) The target of the NSD family of histone lysine methyltransferases depends on the nature of the substrate. J Biol Chem 284: 34283-34295.
11. Kuo A J, Cheung P, Chen K, Zee B M, Kioi M, et al. (2011) NSD2 links dimethylation of histone H3 at lysine 36 to oncogenic programming. Mol Cell 44: 609-620.
12. Martinez-Garcia E, Popovic R, Min D J, Sweet S M, Thomas P M, et al. (2011) The MMSET histone methyl transferase switches global histone methylation and alters gene expression in t(4;14) multiple myeloma cells. Blood 117: 211-220.
13. Wagner E J, Carpenter P B (2012) Understanding the language of Lys36 methylation at histone H3. Nat Rev Mol Cell Biol 13: 115-126.
14. Bergemann A D, Cole F, Hirschhom K (2005) The etiology of Wolf-Hirschhom syndrome. Trends Genet 21: 188-195.
15. Nimura K, Ura K, Shiratori H, Ikawa M, Okabe M, et al. (2009) A histone H3 lysine 36 trimethyltransferase links Nkx2-5 to Wolf-Hirschhorn syndrome. Nature 460: 287-291.
16. Keats J J, Reiman T, Belch A R, Pilarski L M (2006) Ten years and counting: so what do we know about t(4;14)(p16;q32) multiple myeloma. Leuk Lymphoma 47: 2289-2300.
17. Stec I, Wright T J, van Ommen G J, de Boer P A, van Haeringen A, et al. (1998) WHSC1, a 90 kb SET domain-containing gene, expressed in early development and homologous to a Drosophila dysmorphy gene maps in the Wolf-Hirschhom syndrome critical region and is fused to IgH in t(4;14) multiple myeloma. Hum Mol Genet 7: 1071-1082.
18. Keats J J, Reiman T, Maxwell C A, Taylor B J, Larratt L M, et al. (2003) In multiple myeloma, t(4;14)(p16;q32) is an adverse prognostic factor irrespective of FGFR3 expression. Blood 101: 1520-1529.
19. Santra M, Zhan F, Tian E, Barlogie B, Shaughnessy J, Jr. (2003) A subset of multiple myeloma harboring the t(4;14)(p16;q32) translocation lacks FGFR3 expression but maintains an IGH/MMSET fusion transcript. Blood 101: 2374-2376.
20. Ezponda T, Popovic R, Shah M Y, Martinez-Garcia E, Zheng Y, et al. (2012) The histone methyltransferase MMSET/WHSC1 activates TWIST1 to promote an epithelial-mesenchymal transition and invasive properties of prostate cancer. Oncogene.
21. Hudlebusch H R, Santoni-Rugiu E, Simon R, Ralfkiaer E, Rossing H H, et al. (2011) The histone methyltransferase and putative oncoprotein MMSET is overexpressed in a large variety of human tumors. Clin Cancer Res 17: 2919-2933.
22. Zhou P, Wu L L, Wu K M, Jiang W, Li J D, et al. (2013) Overexpression of MMSET is correlation with poor prognosis in hepatocellular carcinoma. Pathol Oncol Res 19: 303-309.
23. Yang S, Zhang Y, Meng F, Liu Y, Xia B, et al. (2013) Overexpression of multiple myeloma SET domain (MMSET) is associated with advanced tumor aggressiveness and poor prognosis in serous ovarian carcinoma. Biomarkers 18: 257-263.
24. Oyer J A, Huang X, Zheng Y, Shim J, Ezponda T, et al. (2013) Point mutation E1099K in MMSET/NSD2 enhances its methyltranferase activity and leads to altered global chromatin methylation in lymphoid malignancies. Leukemia.
25. Lauring J, Abukhdeir A M, Konishi H, Garay J P, Gustin J P, et al. (2008) The multiple myeloma associated MMSET gene contributes to cellular adhesion, clonogenic growth, and tumorigenicity. Blood 111: 856-864.
26. Zheng Y, Sweet S M, Popovic R, Martinez-Garcia E, Tipton J D, et al. (2012) Total kinetic analysis reveals how combinatorial methylation patterns are established on lysines 27 and 36 of histone H3. Proc Natl Acad Sci USA 109: 13549-13554.
27. Bernstein B E, Birney E, Dunham I, Green E D, Gunter C, et al. (2012) An integrated encyclopedia of DNA elements in the human genome. Nature 489: 57-74.
28. Huang N, Kawano M M, Mahmoud M S, Mihara K, Tsujimoto T, et al. (1995) Expression of CD21 antigen on myeloma cells and its involvement in their adhesion to bone marrow stromal cells. Blood 85: 3704-3712.
29. Barski A, Cuddapah S, Cui K, Roh T Y, Schones D E, et al. (2007) High-resolution profiling of histone methylations in the human genome. Cell 129: 823-837.
30. Kondo Y, Shen L, Cheng A S, Ahmed S, Boumber Y, et al. (2008) Gene silencing in cancer by histone H3 lysine 27 trimethylation independent of promoter DNA methylation. Nat Genet 40: 741-750.
31. Bracken A P, Dietrich N, Pasini D, Hansen K H, Helin K (2006) Genome-wide mapping of Polycomb target genes unravels their roles in cell fate transitions. Genes Dev 20: 1123-1136.
32. Subramanian A, Tamayo P, Mootha V K, Mukherjee S, Ebert B L, et al. (2005) Gene set enrichment analysis: a knowledge-based approach for interpreting genome-wide expression profiles. Proc Natl Acad Sci USA 102: 15545-15550.
33. Kamminga L M, Bystrykh L V, de Boer A, Houwer S, Douma J, et al. (2006) The Polycomb group gene Ezh2 prevents hematopoietic stem cell exhaustion. Blood 107: 2170-2179.
34. Schmitges F W, Prusty A B, Faty M, Stutzer A, Lingaraju G M, et al. (2011) Histone methylation by PRC2 is inhibited by active chromatin marks. Mol Cell 42: 330-341.
35. Yuan W, Xu M, Huang C, Liu N, Chen S, et al. (2011) H3K36 methylation antagonizes PRC2-mediated H3K27 methylation. J Biol Chem 286: 7983-7989.
36. Shaffer A L, Wright G, Yang L, Powell J, Ngo V, et al. (2006) A library of gene expression signatures to illuminate normal and pathological lymphoid biology. Immunol Rev 210: 67-85.
37. Granit R Z, Gabai Y, Hadar T, Karamansha Y, Liberman L, et al. (2012) EZH2 promotes a bi-lineage identity in basal-like breast cancer cells. Oncogene.
38. Mendenhall E M, Koche R P, Truong T, Zhou V W, Issac B, et al. (2010) GC-rich sequence elements recruit PRC2 in mammalian ES cells. PLoS Genet 6: e1001244.
39. Verma S K, X. T, LaFrance L V, C. D, Suarez D P, et al. (2012) Identification of Potent, Selective, Cell-Active Inhibitors of the Histone Lysine Methyltransferase EZH2. ACS Medical Chemistry Letters 3: 1091-1096.
40. Baker L A, Allis C D, Wang G G (2008) PHD fingers in human diseases: disorders arising from misinterpreting epigenetic marks. Mutat Res 647: 3-12.
41. Wu H, Zeng H, Lam R, Tempel W, Amaya M F, et al. (2011) Structural and histone binding ability characterizations of human PWWP domains. PLoS One 6: e18919.
42. Kurotaki N, Imaizumi K, Harada N, Masuno M, Kondoh T, et al. (2002) Haploinsufficiency of NSD1 causes Sotos syndrome. Nat Genet 30: 365-366.
43. Mardis E R, Ding L, Dooling D J, Larson D E, McLellan M D, et al. (2009) Recurring mutations found by sequencing an acute myeloid leukemia genome. N Engl J Med 361: 1058-1066.
44. Delhommeau F, Dupont S, Della Valle V, James C, Trannoy S, et al. (2009) Mutation in TET2 in myeloid cancers. N Engl J Med 360: 2289-2301.
45. Dalgliesh G L, Furge K, Greenman C, Chen L, Bignell G, et al. (2010) Systematic sequencing of renal carcinoma reveals inactivation of histone modifying genes. Nature 463: 360-363.
46. Kalushkova A, Fryknas M, Lemaire M, Fristedt C, Agarwal P, et al. (2010) Polycomb target genes are silenced in multiple myeloma. PLoS One 5: e11483.
47. Villa R, Pasini D, Gutierrez A, Morey L, Occhionorelli M, et al. (2007) Role of the polycomb repressive complex 2 in acute promyelocytic leukemia. Cancer Cell 11: 513-525.
48. Peng J C, Valouev A, Swigut T, Zhang J, Zhao Y, et al. (2009) Jarid2/Jumonji coordinates control of PRC2 enzymatic activity and target gene occupancy in pluripotent cells. Cell 139: 1290-1302.
49. Margueron R, Justin N, Ohno K, Sharpe M L, Son J, et al. (2009) Role of the polycomb protein EED in the propagation of repressive histone marks. Nature 461: 762-767.
50. Van Bortle K, Ramos E, Takenaka N, Yang J, Wahi J E, et al. (2012) Drosophila CTCF tandemly aligns with other insulator proteins at the borders of H3K27me3 domains. Genome Res 22: 2176-2187.
51. Gaydos L J, Rechtsteiner A, Egelhofer T A, Carroll C R, Strome S (2012) Antagonism between MES-4 and Polycomb Repressive Complex 2 Promotes Appropriate Gene Expression in C. elegans Germ Cells. Cell Rep.
52. Blackledge N P, Zhou J C, Tolstorukov M Y, Farcas A M, Park P J, et al. (2010) CpG islands recruit a histone H3 lysine 36 demethylase. Mol Cell 38: 179-190.
53. Schwartzentruber J, Korshunov A, Liu X Y, Jones D T, Pfaff E, et al. (2012) Driver mutations in histone H3.3 and chromatin remodelling genes in paediatric glioblastoma. Nature 482: 226-231.
54. Asangani I A, Ateeq B, Cao Q, Dodson L, Pandhi M, et al. (2013) Characterization of the EZH2-MMSET histone methyltransferase regulatory axis in cancer. Mol Cell 49: 80-93.
55. Hollink I H, van den Heuvel-Eibrink M M, Arentsen-Peters S T, Pratcorona M, Abbas S, et al. (2011) NUP98/NSD1 characterizes a novel poor prognostic group in acute myeloid leukemia with a distinct HOX gene expression pattern. Blood 118: 3645-3656.
56. Wang G G, Cai L, Pasillas M P, Kamps M P (2007) NUP98-NSD1 links H3K36 methylation to Hox-A gene activation and leukaemogenesis. Nat Cell Biol 9: 804-812.
57. Faravelli F (2005) NSD1 mutations in Sotos syndrome. Am J Med Genet C Semin Med Genet 137C: 24-31.
58. Zhang J, Ding L, Holmfeldt L, Wu G, Heatley S L, et al. (2012) The genetic basis of early T-cell precursor acute lymphoblastic leukaemia. Nature 481: 157-163.
59. van Haaften G, Dalgliesh G L, Davies H, Chen L, Bignell G, et al. (2009) Somatic mutations of the histone H3K27 demethylase gene UTX in human cancer. Nat Genet 41: 521-523.
60. Croonquist P A, Van Ness B (2005) The polycomb group protein enhancer of zeste homolog 2 (EZH 2) is an oncogene that influences myeloma cell growth and the mutant ras phenotype. Oncogene 24: 6269-6280.
61. Delmore J E, Issa G C, Lemieux M E, Rahl P B, Shi J, et al. (2011) BET bromodomain inhibition as a therapeutic strategy to target c-Myc. Cell 146: 904-917.
62. Creasy C L, McCabe M T, Korenchuk S, Diaz E, Ott H, et al. (2012) A novel selective EZH2 inhibitor exhibits anti-tumor activity in lymphoma with EZH2 activating mutations. Chicago: Proceedings of the 103rd Annual Meeting of the American Association for Cancer Research. pp. 492.
63. McCabe M T, Ott H M, Ganji G, Korenchuk S, Thompson C, et al. (2012) EZH2 inhibition as a therapeutic strategy for lymphoma with EZH2-activating mutations. Nature.
64. Giannopoulou E G, Elemento O (2011) An integrated ChIP-seq analysis platform with customizable workflows. BMC Bioinformatics 12: 277.
65. Elemento O, Slonim N, Tavazoie S (2007) A universal framework for regulatory element discovery across all genomes and data types. Mol Cell 28: 337-350.
66. Heinz S, Benner C, Spann N, Bertolino E, Lin Y C, et al. (2010) Simple combinations of lineage-determining transcription factors prime cis-regulatory elements required for macrophage and B cell identities. Mol Cell 38: 576-589.
67. Goodarzi H, Elemento O, Tavazoie S (2009) Revealing global regulatory perturbations across human cancers. Mol Cell 36: 900-911.

1. A method of treating cancer in a human in need thereof, comprising determining at least one of the following in a sample from said human:
a. the presence of an increased level of MMSET as compared to a control; or b. the presence or absence of a decreased level of a functional UTX protein as compared to a control; and
administering to said human an effective amount of an EZH2 inhibitor or pharmaceutically acceptable salt thereof if there is an increased level of MMSET as compared to a control or there is a decreased level of a functional UTX protein as compared to a control, or both.

2. The method of claim 1, wherein the EZH2 inhibitor is a compound of Formula (I): 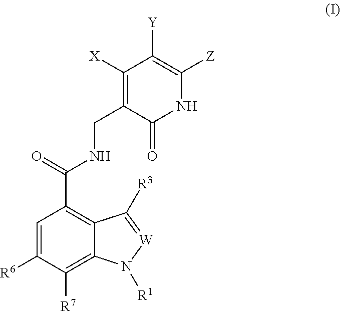
wherein:
W is N or CR²;
X and Z are each independently selected from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halogen, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
Y is hydrogen or halogen;
R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₂-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, or —CONR^{a}NR^{a}R^{b};
When present R² is hydrogen, (C₁-C₈)alkyl, trifluoromethyl, alkoxy, or halogen, in which said (C₁-C₈)alkyl may be substituted with one to two groups selected from amino and (C₁-C₃)alkylamino;
R⁷ is hydrogen, (C₁-C₃)alkyl, or alkoxy; R³ is hydrogen, (C₁-C₈)alkyl, cyano, trifluoromethyl, —NR^{a}R^{b}, or halogen;
R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halogen, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};
each R^{c} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};
R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);
or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2, or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;
or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;
or a pharmaceutically acceptable salt thereof.

3. The method of claim 1, wherein the EZH2 inhibitor is Compound B: 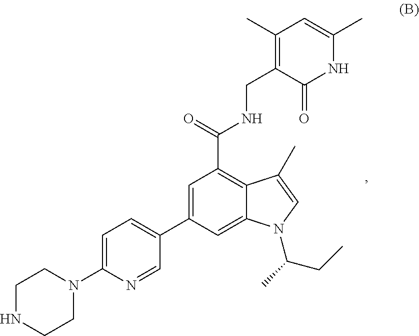 [SMILES: CC[C@H](C)n1cc(C)c2c(C(=O)NCc3c(C)cc(C)[nH]c3=O)cc(-c3ccc(N4CCNCC4)nc3)cc21]
or a pharmaceutically acceptable salt or preparation thereof.

4. The method of claim 1 wherein the EZH2 Inhibitor is Compound C: 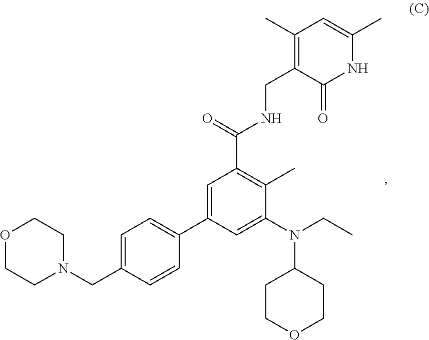 [SMILES: CCN(c1cc(-c2ccc(CN3CCOCC3)cc2)cc(C(=O)NCc2c(C)cc(C)[nH]c2=O)c1C)C1CCOCC1]
or a pharmaceutically acceptable salt or preparation thereof.

5. The method of claim 1, wherein the EZH2 inhibitor is N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide, or a pharmaceutically acceptable salt or preparation thereof.

6. A pharmaceutical composition comprising an EZH2 inhibitor of Formula I, Compound B, or Compound C for use in treatment of cancer, wherein the cancer is characterized as having one or both of the following:
the presence of increased level in MMSET expression as compared to a control; or the presence of a decreased level of a functional UTX protein as compared to a control.

7. The method or composition of claim 1, wherein the sample comprises at least one cancer cell.

8. The method or composition of claim 1, wherein the cancer is a myeloid malignancy.

9. The method or composition of claim 1, wherein the cancer is myeloma.

10. The method or composition of claim 1, wherein the cancer is myeloma and the myeloma is multiple myeloma.

11. The method or composition of claim 1, wherein the cancer is multiple myeloma and the multiple myeloma is a t(4;14)+ multiple myeloma.

12. The method or composition of claim 1, wherein the cancer is lymphoma.

13. The method or composition of claim 1, wherein the cancer is a solid tumor.

14. The method or composition of claim 13, wherein the solid tumor cancer is selected from the group consisting of prostate cancer, bladder cancer, lung cancer, and skin cancer.

15. The method or composition of claim 1, further comprising administering one or more additional anti-neoplastic agents.

16. A kit for the treatment of cancer comprising a kit for determining one or more of a-b of claim 1, and a means for determining one or more of a-b of claim 1.

17. The kit of claim 16, wherein said means is selected from the group consisting of primers, probes, and antibodies.
